(19) United States
(12) Patent Application Publication
(10) Pub. No.: US 2013/0109737 A1
(43) Pub. Date: May 2, 2013

(54) MEDIATOR AND COHESIN CONNECT GENE EXPRESSION AND CHROMATIN ARCHITECTURE

(71) Applicants: Richard A. Young, Weston, MA (US); Jamie J. Newman, Cambridge, MA (US); Michael H. Kagey, Somerville, MA (US); Steve Bilodeau, Cambridge, MA (US)

(72) Inventors: Richard A. Young, Weston, MA (US); Jamie J. Newman, Cambridge, MA (US); Michael H. Kagey, Somerville, MA (US); Steve Bilodeau, Cambridge, MA (US)

(21) Appl. No.: 13/578,114

(22) PCT Filed: Feb. 9, 2011
(86) PCT No.: PCT/US11/24257

§ 371 Date: Nov. 27, 2012

(30) Foreign Application Priority Data
Feb. 9, 2010 (US) 61302907
Feb. 11, 2010 (US) 61303569
Aug. 18, 2010 (US) 61401823

(51) Int. Cl. C12Q 1/68; C12N 9/96

(52) U.S. Cl. CPC C12Q 1/6811; C12Q 1/6804; C12N 9/96
USPC 514/44 A; 506/9; 435/6.12; 435/6.19; 435/6.13; 435/188; 435/7.4; 435/375

(57) ABSTRACT

In some aspects, the present invention provides compositions and methods relating at least in part to modulation of the Cohesin-Mediator interaction. The invention provides compositions and methods useful for modulating Cohesin-Mediator function. The invention further provides compositions and methods useful for identifying compounds that modulate Cohesin-Mediator function. In some aspects, the invention provides compositions and methods useful for treating a disorder involving altered Cohesin-Mediator function.

### RELATED APPLICATIONS

This application claims priority to and the benefit of U.S. Application No. 61/302,907, filed Feb. 9, 2010, U.S. Application No. 61/303,569, filed Feb. 11, 2010, and U.S. Application No. 61/401,823, filed Aug. 18, 2010. The entire contents of these applications are incorporated herein by reference.

### GOVERNMENT SUPPORT

The invention was supported, in whole or in part, by grant HG002668 from the National Institutes of Health. The U.S. Government has certain rights in the invention.

### BACKGROUND OF THE INVENTION

Transcription factors regulate cell-specific gene expression programs. These factors frequently bind to enhancer elements that can be located some distance from the core promoter elements where the transcription initiation apparatus is bound. A better understanding of the interaction between enhancer-bound transcription factors and the transcription apparatus at the core promoter would be of significant interest for a broad range of applications.

### SUMMARY OF THE INVENTION

The present invention relates in part to the discovery that the protein complexes Cohesin and Mediator co-occupy the enhancers and core promoters of active genes in embryonic stem (ES) cells and other cells and are necessary for normal transcriptional activity and maintenance of ES cell state. The invention also relates in part to the discovery that Cohesin and Mediator tend to co-occupy cell-type specific genes in mammalian cells. Aspects of the invention further relate to the discovery that Cohesin and Mediator physically interact in mammalian cells and create a stable, looped chromatin structure at active promoters throughout the genome, thus generating cell-type specific chromatin architecture.

In some aspects, the invention provides a method of identifying a compound that modulates the interaction between Cohesin and Mediator comprising: (a) contacting a composition comprising at least one Cohesin component and at least one Mediator component with a test compound; (b) assessing the level of interaction between Cohesin and Mediator that occurs in the composition; and (c) comparing the level of interaction measured in step (b) with a suitable reference value, wherein if the level of interaction measured in step (b) differs from the reference value, the test compound modulates the interaction between Cohesin and Mediator. In some embodiments, the at least one Cohesin component comprises an Smc1a, Smc3, or Nipb1 polypeptide. In some embodiments, the at least one Cohesin component comprises an Smc1a, Smc3, and Nipb1 polypeptide. In some embodiments, the at least one Mediator component comprises a Med1 or a Med12 polypeptide. In some embodiments, the at least one Mediator component comprises Med6, Med7, Med10, Med12, Med14, Med15, Med17, Med21, Med24, Med27, Med28 and Med30 polypeptides. In some embodiments, the Cohesin component and the Mediator component are contacted with the test compound within a cell. In some embodiments, the reference value is a value obtained in the absence of the test compound. In some embodiments, the level of interaction is measured by a method comprising: (i) isolating the Cohesin component or the Mediator component under conditions suitable for maintaining a Cohesin-Mediator interaction; and (ii) measuring the extent to which isolating the Cohesin component results in isolating at least one Mediator component or measuring the extent to which isolating the Mediator component results in isolating at least one Cohesin component. In some embodiments, isolating the Cohesin component or the Mediator component comprises contacting the composition with an agent that specifically binds to the Cohesin component or the Mediator component, respectively. In some embodiments, the level of interaction is measured by assessing expression of a gene whose expression depends at least in part on a Cohesin-Mediator complex. In some embodiments the level of interaction is measured by detecting a DNA loop formed by Mediator and Cohesin. In some embodiments the level of interaction is measured by detecting co-occupancy of a promoter or enhancer by Mediator and Cohesin. In some embodiments the Cohesin component and the Mediator component are contacted with the test compound within a pluripotent cell, and the level of interaction is measured by detecting a loss of pluripotency (LOP) phenotype of the cell, wherein the LOP phenotype indicates that the compound disrupts interaction between Cohesin and Mediator. In some embodiments the Cohesin component or the Mediator component is a variant Cohesin component or a variant Mediator component. In some embodiments the Cohesin component or the Mediator component is a variant Cohesin component or a variant Mediator component and the variant Cohesin component or variant Mediator component is associated with a disorder. In some embodiments, if the test compound modulates the interaction between Cohesin and Mediator, the test compound is a candidate compound for treatment of a disorder. In some embodiments, the Cohesin component or the Mediator component is from a cell derived from a subject having the disorder. In some embodiments, the Cohesin component or the Mediator component is a variant Cohesin component or a variant Mediator component, and the variant Cohesin component or variant Mediator component is associated with a disorder. In some embodiments, the disorder is associated with mutations in a gene that encodes a Cohesin component or a Mediator component. In some embodiments the disorder is a developmental disorder. In some embodiments the disorder is a proliferative disorder.

In another aspect, the invention provides a method of identifying a compound that affects cell state comprising the step of: identifying a compound that modulates the interaction between Cohesin and Mediator. In some embodiments the cell state is characteristic of a cell type of interest, and the method comprises identifying a compound that modulates the interaction between Cohesin and Mediator in a cell of that cell type. In some embodiments the cell state is characteristic of or associated with a disorder. In some embodiments, the cell state is characteristic of or associated with a disorder and the method comprises identifying a compound that modulates the interaction between Cohesin and Mediator in a cell derived from a subject having the disorder. In some embodiments the cell state is characteristic of or associated with a disorder, and a compound identified as modulating the interaction between Cohesin and Mediator is a candidate compound for treating the disorder. In some embodiments the disorder is associated with mutations in a gene that encodes a Cohesin component or a Mediator component. In some embodiments the disorder is a developmental disorder. In some embodiments the disorder is a proliferative disorder. In some embodiments the cell state is characteristic of a cell type of interest, and the composition comprises a Cohesin component or a Mediator component from a cell of that type. In some embodiments the cell state is characteristic of a cell type of interest, and the composition comprises a cell-type specific transcription factor whose expression is characteristic of the cell type of interest. In some embodiments the Cohesin and Mediator components are contacted with the test compound within a cell of the cell type of interest. In some embodiments the Cohesin component or the Mediator component is from a cell derived from a subject suffering from a disorder of interest. In some embodiments the Cohesin component or the Mediator component is from a cell derived from a subject having a disorder of interest, wherein the disorder is a developmental disorder. In some embodiments the Cohesin component or the Mediator component is from a cell derived from a subject having a disorder of interest, wherein the disorder is a proliferative disorder. In some embodiments the cell state is characteristic of or associated with a disorder, and the composition comprises a Cohesin component and a Mediator component from a cell derived from a subject having the disorder. In some embodiments the cell state is characteristic of or associated with a disorder, and wherein a compound identified as modulating the interaction between Cohesin and Mediator is further identified as a candidate compound for treating the disorder.

In another aspect, the invention provides a method of identifying a compound that modulates the function of a Cohesin-Mediator complex comprising steps of: (a) contacting a composition comprising at least one Cohesin component and at least one Mediator component with a test compound; (b) assessing at least one function of a Cohesin-Mediator complex; and (c) comparing the function measured in step (b) with a suitable reference value, wherein if the function measured in step (b) differs from the reference value, the test compound modulates function of a Cohesin-Mediator complex. In some embodiments the at least one Cohesin component comprises an Smc1 or Smc3 polypeptide. In some embodiments the at least one Cohesin component comprises an Smc1 polypeptide, an Smc3 polypeptide, and a Nibp1 polypeptide. In some embodiments the at least one Cohesin component comprises an Smc1 polypeptide, an Smc3 polypeptide, a STAG polypeptide, and a Nibp1 polypeptide. In some embodiments the at least one Mediator component comprises a Med1 or a Med12 polypeptide. In some embodiments the at least one Mediator component comprises Med6, Med7, Med10, Med12, Med14, Med15, Med17, Med21, Med24, Med27, Med28 and Med30 polypeptides. In some embodiments the Cohesin component and the Mediator component are contacted with the test compound within a cell. In some embodiments the composition comprises a Cohesin complex and a Mediator complex. In some embodiments the reference value is a value obtained in the absence of the test compound. In some embodiments the function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In some embodiments the function is measured by assessing expression of a gene whose expression depends at least in part on a Cohesin-Mediator complex. In some embodiments the function is measured by detecting a DNA loop formed by Mediator and Cohesin. In some embodiments the function is measured by detecting co-occupancy of a promoter or enhancer by Mediator and Cohesin. In some embodiments the Cohesin component and the Mediator component are contacted with the test compound within a pluripotent cell, and the function is measured by detecting a loss of pluripotency (LOP) phenotype of the cell, wherein the LOP phenotype indicates that the compound modulates function of a Cohesin-Mediator complex. In some embodiments the Cohesin component or the Mediator component is a variant Cohesin component or a variant Mediator component. In some embodiments the Cohesin component or the Mediator component is a variant Cohesin component or a variant Mediator component and the variant Cohesin component or variant Mediator component is associated with a disorder. In some embodiments, if the test compound modulates the interaction between Cohesin and Mediator, the test compound is a candidate compound for treatment of a disorder. In some embodiments the Cohesin component or the Mediator component is from a cell derived from a subject having the disorder. In some embodiments the Cohesin component or the Mediator component is a variant Cohesin component or a variant Mediator component, and the variant Cohesin component or variant Mediator component is associated with a disorder. In some embodiments the disorder is associated with mutations in a gene that encodes a Cohesin component or a Mediator component. In some embodiments the disorder is a developmental disorder. In some embodiments the disorder is a proliferative disorder.

In another aspect, the invention provides a method of identifying a compound that affects cell state comprising the step of: identifying a compound that modulates a function of a Cohesin-Mediator complex. In some embodiments the compound modulates the interaction between Cohesin and Mediator. In some embodiments the function is selected from the group consisting of (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In some embodiments the cell state is characteristic of a cell type of interest, and the method comprises identifying a compound that modulates function of a Cohesin-Mediator complex, wherein the compound optionally modulates the interaction between Cohesin and Mediator. In some embodiments the cell state is characteristic of or associated with a disorder. In some embodiments the cell state is characteristic of or associated with a disorder and the method comprises identifying a compound that modulates the interaction between Cohesin and Mediator in a cell derived from a subject having the disorder. In some embodiments the cell state is characteristic of or associated with a disorder, and wherein a compound identified as modulating the interaction between Cohesin and Mediator is a candidate compound for treating the disorder. In some embodiments the disorder is associated with mutations in a gene that encodes a Cohesin component or a Mediator component. In some embodiments the disorder is a developmental disorder. In some embodiments the disorder is a proliferative disorder. In some embodiments the cell state is characteristic of a cell type of interest, and the composition comprises a Cohesin component or a Mediator component from a cell of that type. In some embodiments the cell state is characteristic of a cell type of interest, and the composition comprises a cell-type specific transcription factor whose expression is characteristic of the cell type of interest. In some embodiments the Cohesin and Mediator components are contacted with the test compound within a cell of the cell type of interest. In some embodiments the Cohesin component or the Mediator component is from a cell derived from a subject suffering from a disorder of interest. In some embodiments the Cohesin component or the Mediator component is from a cell derived from a subject having a disorder of interest, wherein the disorder is a developmental disorder. In some embodiments the Cohesin component or the Mediator component is from a cell derived from a subject having a disorder of interest, wherein the disorder is a proliferative disorder. In some embodiments the cell state is characteristic of a disorder, and the composition comprises a Cohesin component and a Mediator component from a cell derived from a subject having the disorder. In some embodiments the cell state is characteristic of a disorder, and wherein a compound identified as modulating the interaction between Cohesin and Mediator is further identified as a candidate compound for treating the disorder.

In another aspect, the invention provides a method of identifying a candidate compound for treatment of a disorder comprising the step of: identifying a compound that modulates the function of a Cohesin-Mediator complex. In some embodiments the compound modulates an interaction between Cohesin and Mediator. In some embodiments the function is selected from the group consisting of (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In some embodiments the disorder is associated with mutations in a gene that encodes a Cohesin component or a Mediator component. In some embodiments the disorder is a developmental disorder. In some embodiments the disorder is a proliferative disorder.

In another aspect, the invention provides a method of identifying a compound that modifies chromatin architecture comprising the step of: identifying a compound that modulates the function of a Cohesin-Mediator complex. In some embodiments the compound modulates interaction between a Cohesin component and a Mediator component. In some embodiments the function comprises an interaction between Mediator and Cohesin or components thereof. In some embodiments the compound modifies chromatin architecture in a cell-type specific manner.

In another aspect, the invention provides a method of identifying a compound that affects cell state comprising: (a) providing a pluripotent cell that expresses a maintenance of pluripotency (MOP) gene, wherein the MOP gene is a gene whose inhibition results in at least one phenotype indicative of loss of pluripotency (LOP phenotype); (b) contacting the cell with a test compound; (c) inhibiting the MOP gene; (d) determining whether the cell exhibits at least one LOP phenotype, wherein if the cell fails to exhibit at least one LOP phenotype as compared to a suitable control, the compound affects cell state. In some embodiments the MOP gene is a gene listed in Table S2. In some embodiments the LOP phenotype of step (a) is selected from the group consisting of: (i) reduced levels of at least one transcription factor associated with ES cell pluripotency; (ii) a loss of pluripotent cell colony morphology; (iii) reduced levels of mRNAs specifying at least one transcription factor associated with ES cell pluripotency; (iv) increased expression of mRNAs encoding at least 3 developmentally important transcription factors. In some embodiments the LOP phenotype of step (d) is selected from the group consisting of: (i) reduced levels of at least one transcription factor associated with ES cell pluripotency; (ii) a loss of pluripotent cell colony morphology; (iii) reduced levels of mRNAs specifying at least one transcription factor associated with ES cell pluripotency; (iii) increased expression of mRNAs encoding at least 3 developmentally important transcription factors. In some embodiments the LOP phenotype of step (a) and step (d) are the same. In some embodiments the LOP phenotype of step (a), step (d), or both, is expression of Oct 4 protein. In some embodiments the at least one transcription factor associated with pluripotency is selected from the group consisting of Oct 4, Nanog, and Sox2. In some embodiments the cell is an ES cell. In some embodiments the cell comprises a nucleic acid that encodes a shRNA targeted to the MOP gene, wherein expression of the shRNA is inducible, and wherein inhibiting the MOP gene comprises inducing expression of the shRNA. In some embodiments the MOP gene encodes a Cohesin component. In some embodiments the MOP gene encodes a Mediator component. In some embodiments mutations in the MOP gene, or mutations in a gene that encodes a product which interacts with the product encoded by the MOP gene, are associated with a disorder. In some embodiments the disorder is a developmental disorder. In some embodiments the disorder is a hereditary disorder. In some embodiments the MOP gene encodes a Cohesin component. In some embodiments the MOP gene encodes a Mediator component. In some embodiments the compound is a candidate compound for treating the disorder. In some embodiments the MOP gene encodes a Cohesin component. In some embodiments the MOP gene encodes a Mediator component. In some embodiments the MOP gene encodes Nipb1. In some embodiments the disorder is Cornelia de Lange syndrome. In some embodiments the MOP gene encodes Nipb1 and the disorder is Cornelia de Lange syndrome. In some embodiments the MOP gene encodes Med12. In some embodiments the disorder is Opitz-Kaveggia (FG) syndrome, Lujan syndrome, schizophrenia or congenital heart failure. In some embodiments the MOP gene encodes Med12 and the disorder is Opitz-Kaveggia (FG) syndrome, Lujan syndrome, schizophrenia or congenital heart failure. In another aspect, the invention provides isolated complex comprising a Cohesin component and a Mediator component. In some embodiments the complex is substantially free of CTCF. In some embodiments the Cohesin component or the Mediator component is a variant Cohesin component or a variant Mediator component, respectively. In some embodiments the complex is isolated from a cell derived from a subject who has a disorder of interest. In some embodiments the Cohesin component or the Mediator component is a recombinant protein. In some embodiments the Cohesin component or the Mediator component comprises a tag. In some embodiments, the complex further comprises a cell-type specific transcription factor. In some embodiments, the complex further comprises a DNA loop. In some embodiments, the complex comprises a Nipb1 polypeptide. In some embodiments, the complex comprises a Nipb1 polypeptide, a STAG polypeptide, and an Smc polypeptide. In some embodiments, the complex comprises a Nipb1 polypeptide, a STAG polypeptide, an Smc1a polypeptide, and Smc3 polypeptide. In some embodiments, the complex comprises multiple Mediator components. In another aspect, the invention provides a composition comprising any of the above-mentioned isolated complexes, wherein the composition is substantially free of Cohesin components that are not complexed with Mediator components. In some embodiments, the composition is substantially free of CTCF. In some embodiments, the composition is substantially free of Mediator components not complexed with Cohesin components. In another aspect, the invention provides a method of characterizing a cell comprising: (a) isolating material comprising a Mediator component from a cell using an agent that binds to Mediator or that binds to a Mediator-associated protein; and (b) detecting a Cohesin component in the isolated material. In some embodiments the method further comprises analyzing a Cohesin component present in the isolated material. In some embodiments the Mediator component or the Cohesin component is a variant Mediator component or a variant Cohesin component, respectively. In some embodiments the Cohesin component or the Mediator component is a recombinant protein. In some embodiments the Cohesin component or the Mediator component comprises a tag. In some embodiments the cell is derived from a subject having or suspected of having a disorder of interest. In some embodiments the cell is derived from a subject having or suspected of having a disorder of interest and the method further comprises analyzing a Cohesin component present in the isolated material. In some embodiments the cell is derived from a subject having or suspected of having a disorder of interest and the method further comprises diagnosing the subject as having or not having the disorder based at least in part on the amount or properties of a Cohesin component present in the isolated material. In some embodiments the invention provides a method of characterizing a cell comprising: (a) isolating a complex comprising a Cohesin component from a cell using an agent that binds to Cohesin or that binds to a Cohesin-associated protein; and (b) detecting a Mediator component in the complex. In some embodiments, the method further comprises analyzing a Mediator component present in the isolated material. In some embodiments, the Mediator component or the Cohesin component is a variant Mediator component or a variant Cohesin component, respectively. In some embodiments, the Cohesin component or the Mediator component is a recombinant protein. In some embodiments, the Cohesin component or the Mediator component comprises a tag. In some embodiments, the cell is derived from a subject having or suspected of having a disorder of interest. In some embodiments the cell is derived from a subject having or suspected of having a disorder of interest and the method further comprises analyzing a Mediator component present in the isolated material. In some embodiments the cell is derived from a subject having or suspected of having a disorder of interest and the method further comprises diagnosing the subject as having or not having the disorder based at least in part on the amount or properties of the Mediator component detected.

In another aspect, the invention provides a method of characterizing a cell derived from a subject having or suspected of having a Cohesin-associated disorder comprising the step of determining whether the cell has an alteration in a Mediator component as compared with a reference. In some embodiments the method comprises determining whether the cell has a mutation in a gene encoding a Mediator component. In some embodiments the method comprises determining whether the cell has increased or decreased expression or post-translational modification of a Mediator component. In some embodiments the method comprises determining whether the cell has altered binding of Mediator to at least one enhancer or promoter. In some embodiments the method comprises determining whether the cell has altered interaction between Mediator and Cohesin.

In another aspect, the invention provides a method of characterizing a cell derived from a subject having or suspected of having a Mediator-associated disorder comprising the step of determining whether the cell has an alteration in a Cohesin component as compared with a reference. In some embodiments the method comprises determining whether the cell has a mutation in a gene encoding a Cohesin component. In some embodiments the method comprises determining whether the cell has increased or decreased expression or post-translational modification of a Cohesin component. In some embodiments the method comprises determining whether the cell has altered binding of Cohesin to at least one enhancer or promoter. In some embodiments the method comprises determining whether the cell has altered interaction between Mediator and Cohesin.

In another aspect, the invention provides a method of characterizing a cell comprising: analyzing a function of a Cohesin-Mediator complex of the cell. In some embodiments the cell is derived from a subject having a disorder of interest. In some embodiments the cell is derived from a subject having or suspected of having a Mediator-associated disorder. In some embodiments the cell is derived from a subject having or suspected of having a Cohesin-associated disorder. In some embodiments the method comprises determining whether the cell has altered function of a Cohesin-Mediator complex as compared with a reference. In some embodiments the function is selected from the group consisting of: (a) binding of a Cohesin complex to a Mediator complex; (b) occupancy of a cell type specific gene; (c) controlling expression or activity of a cell type specific gene; and (d) mediating response to a signal transduction pathway.

In another aspect, the invention provides a method of modifying cell state comprising: modulating a Cohesin-Mediator function in the cell, thereby modifying cell state. In some embodiments the method comprises contacting a cell with a compound that modulates a Cohesin-Mediator function, thereby modifying cell state. In some embodiments the function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In some embodiments the state is a state characteristic of or associated with a disorder. In some embodiments the cell is in a proliferative state prior to being contacted with the compound. In some embodiments the cell is in a subject. In some embodiments the method comprises administering a compound to a subject, wherein the compound modulates a Cohesin-Mediator function. In some embodiments the method comprises administering a compound to a subject, wherein the compound modulates a Cohesin-Mediator function, and wherein the modulation treats a disorder.

In another aspect, the invention provides a method of treating a subject in need of treatment for a disorder associated with decreased function of a transcription-specific Cohesin complex, the method comprising administering a compound that increases transcriptional activation activity of Mediator to the subject. In some embodiments the subject has a mutation in a gene encoding Smca1, Smc3, or Nipb1. In some embodiments the subject suffers from Cornelia deLange syndrome.

The practice of the present invention will typically employ, unless otherwise indicated, conventional techniques of molecular biology, cell culture, recombinant nucleic acid (e.g., DNA) technology, immunology, nucleic acid and polypeptide synthesis, detection, manipulation, and quantification, and RNA interference that are within the skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information relating to therapeutic agents and human diseases may be found in Goodman and Gilman's The Pharmacological Basis of Therapeutics, 11th Ed., McGraw Hill, 2005 or 12^{th} Ed, 2010; Katzung, B. (ed.) Basic and Clinical Pharmacology, McGraw-Hill/Appleton & Lange; 10th ed. (2006) or 11th edition (July 2009). Information relating to cancer may be found in Cancer: Principles and Practice of Oncology (V. T. De Vita et al., eds., J. B. Lippincott Company, 7th ed., 2004 or 8th ed., 2008) and Weinberg, R A, The Biology of Cancer, Garland Science, 2006.

### BRIEF DESCRIPTION OF THE DRAWING

FIG. 1 Mediator and cohesin contribute to the ES cell state. a, Mediator and cohesin components were highly represented in an shRNA screen for regulators of ES cell state. Complete results are listed in Supplementary Tables 1 and 2. b, Knockdown of mediator (Med12), cohesin (Smc1a) or Nipb1 caused reduced Oct4 protein levels and changes in ES cell colony morphology. Murine ES cells were infected with GFP control, Med12, Smc1a or Nipb1 shRNAs, and stained for Oct4 and with Hoechst. Scale bar, 100 μm. c, Mediator, cohesin and Nipb1 knockdowns all cause reduced expression of ES cell regulators and increased expression of developmental regulators. ES cells were infected with the indicated shRNA and gene expression levels relative to a control GFP infection were determined with microarrays. Log₂ fold expression changes were rank ordered from lowest to highest for all genes.

FIG. 2 Genome-wide occupancy of mediator and cohesin in ES cells. a, Binding profiles for ES cell transcription factors (Oct4, Nanog and Sox2), mediator (Med1 and Med12), cohesin (Smc1a, Smc3 and Nipb1), CTCF and components of the transcription apparatus (Pol2 and TBP) at the Oct4 and Nanog loci. ChIP-Seq data are shown in reads per million with they axis floor set to 0.5 reads per million. Oct4/Sox2, CTCF and TBP (TATA box) sequence motifs are indicated. b, Venn diagram showing the overlap of high-confidence (P<10⁻⁹) cohesin (Smc1a) occupied sites with those bound by CTCF, mediator (Med12) and Nipb1. c, Region map showing that Smc1a, Nipb1 and Med12 co-occupied sites generally occur in close proximity to Pol2 and in the absence of CTCF. For each Smc1a occupied region, the occupancy of Med12, Nipb1, Pol2 and CTCF is indicated within a 10-kb window centred on the Smc1a region. d, Heat map indicating that regions co-occupied by Smc1a, Med12 and Nipb1, which are associated with active genes, exhibit similar expression changes with knockdown of Smc1a, Med12 or Nipb1, Log₂ expression data were ordered based on the Smc1a knockdown data and are shown for all Smc1a, Med12 and Nipb1 co-occupied regions that could be mapped to a gene, as described in Supplementary Information.

FIG. 3 Mediator and cohesin interact. a, Mediator (Med23) is detected by western blot (WB) when crosslinked, sheared chromatin is subjected to immunoprecipitation with antibodies against mediator (Med1, Med12) or cohesin (Smc1a, Smc3). WCE, whole-cell extract. b, Cohesin (Smc1a, Smc3) and mediator (Med23) are detected by western blot after immunoprecipitation of uncrosslinked ES cell nuclear extracts (NE) with a Nipb1 antibody. c, Cohesin (Smc3) and Nipb1 co-purify with mediator. The input fractions and immunoprecipitated eluate (IP Eluate) were examined by western blot and silver staining. Molecular weight (MW) markers (kDa) are shown.

FIG. 4 Mediator and cohesin binding profiles predict enhancer-promoter looping events. a-d, A looping event was detected between the upstream enhancer and the core promoter of Nanog (a), Phc1 (b), Oct4 (c) and Lefty1 (d) by 3C in ES cells, but not in MEFs. ES cell and MEF crosslinked chromatin was digested by MspI or HaeIII and religated under conditions that favour intramolecular ligation events. The interaction frequency between the anchoring point and distal fragments was determined by PCR and normalized to BAC templates and control regions. Error bars represent the standard error of the average of 3 independent PCR reactions. ChIP-Seq data for Med12, Smc1a and Nipb1 are shown in reads per million with they axis floor set to 0.5 reads per million. Restriction enzyme sites are indicated above the 3C graph. The genomic coordinates are build NCBI36/mm8. Biological replicates of the 3C experiments and the full 3C profile are presented in Supplementary FIG. 7.

FIG. 5 Cell-type-specific occupancy of mediator and cohesin. a, Region map of a 10-kb window around mediator and cohesin co-occupied sites for murine ES cells (mES; Smc1a and Med12) and MEFs (Smc1a and Med1) indicates that co-occupied regions are different between the cell types. b, Region map of a 10-kb window around cohesin (Smc1a) and CTCF co-occupied sites indicates that many of these regions are co-occupied in ES cells and in MEFs. c, Western blot of ES and MEF cell extracts indicates that cohesin protein levels are similar for both cell types, whereas mediator protein levels are substantially lower in MEFs.

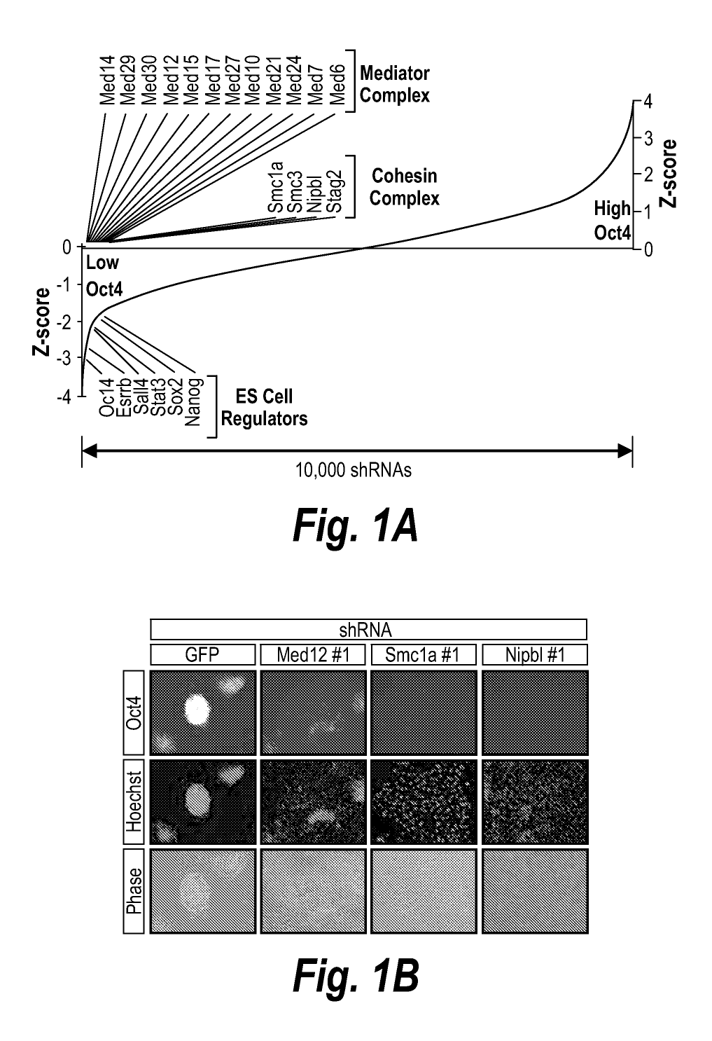

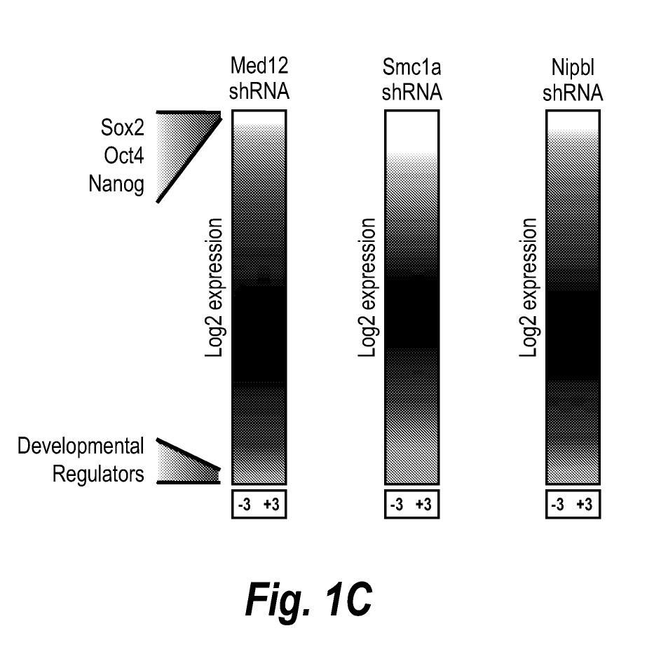

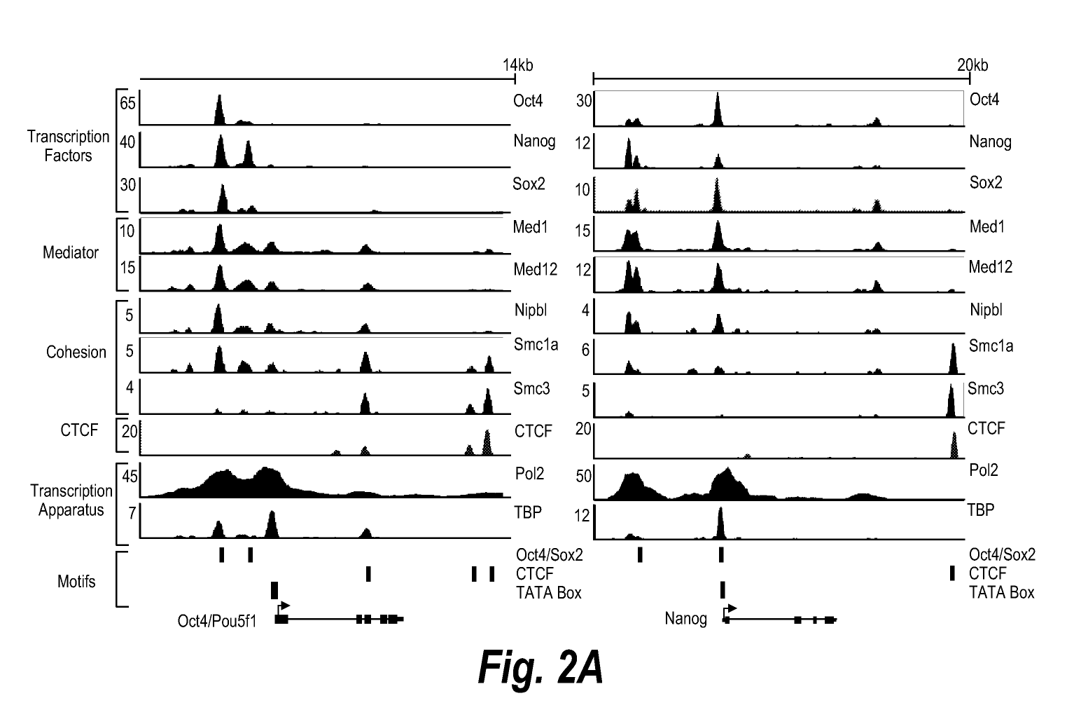

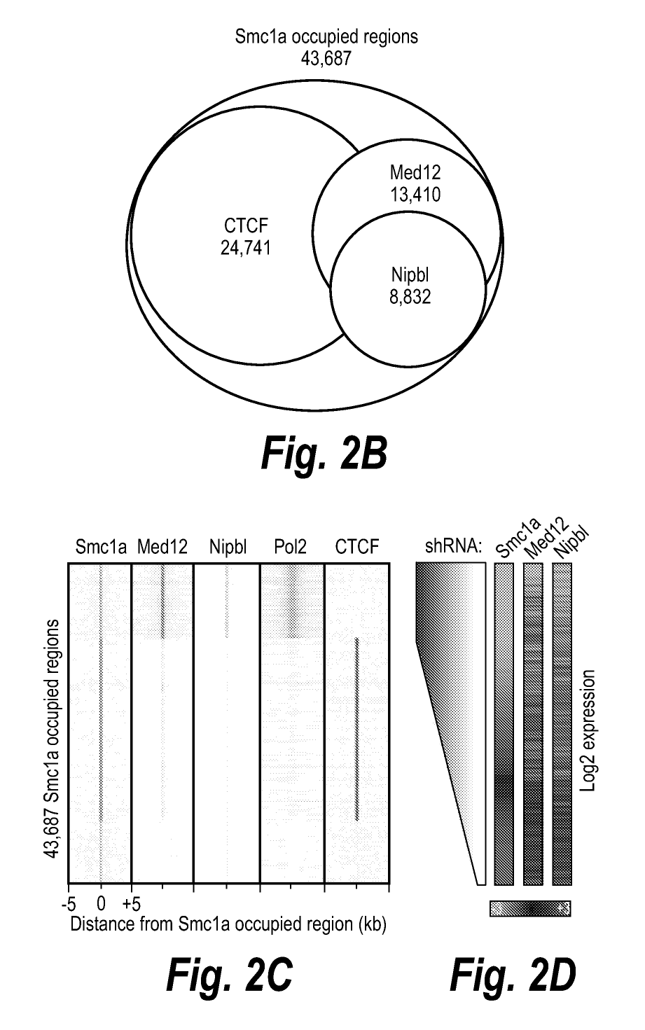

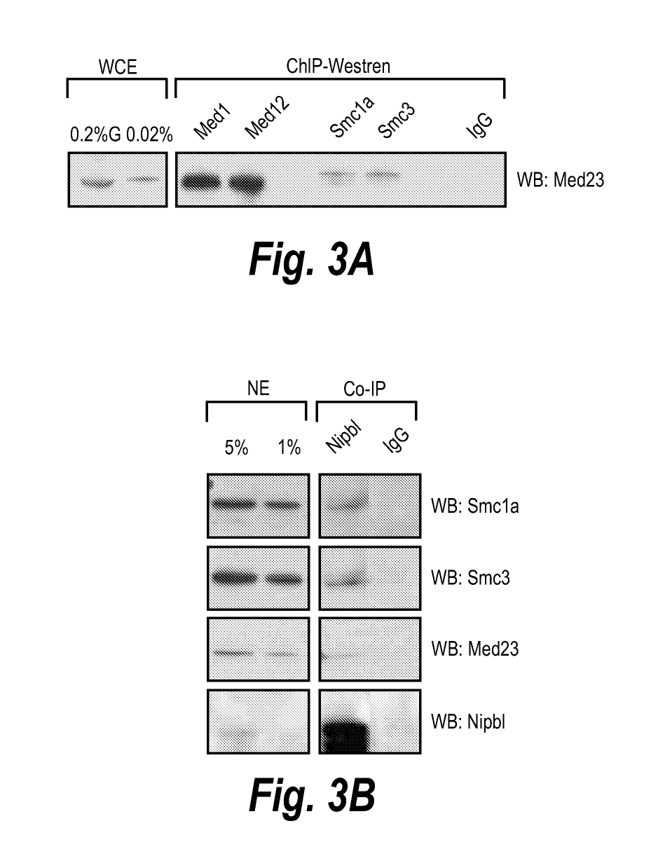

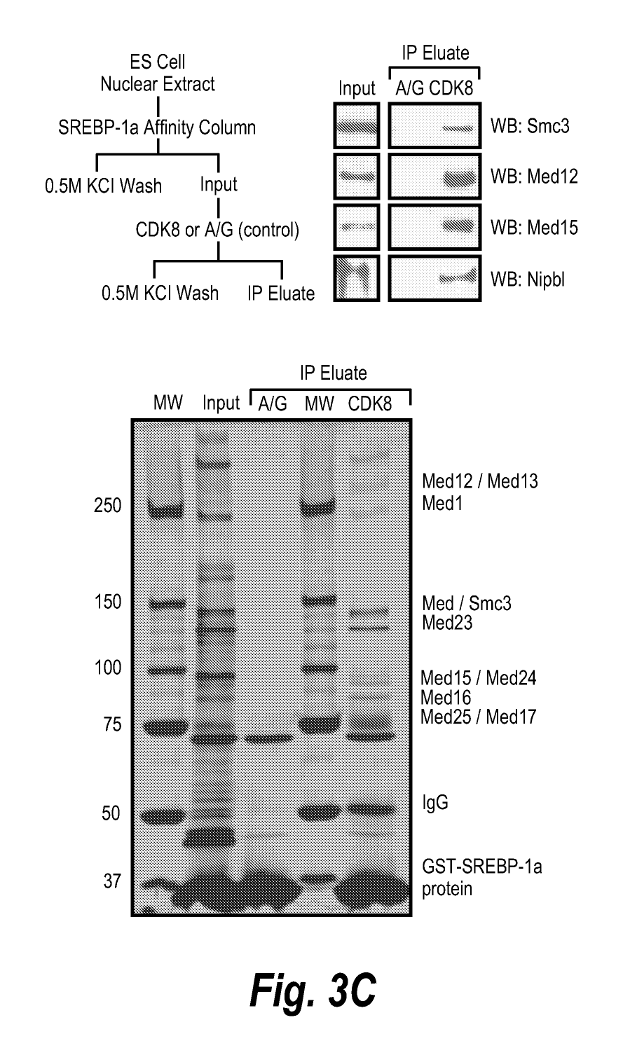

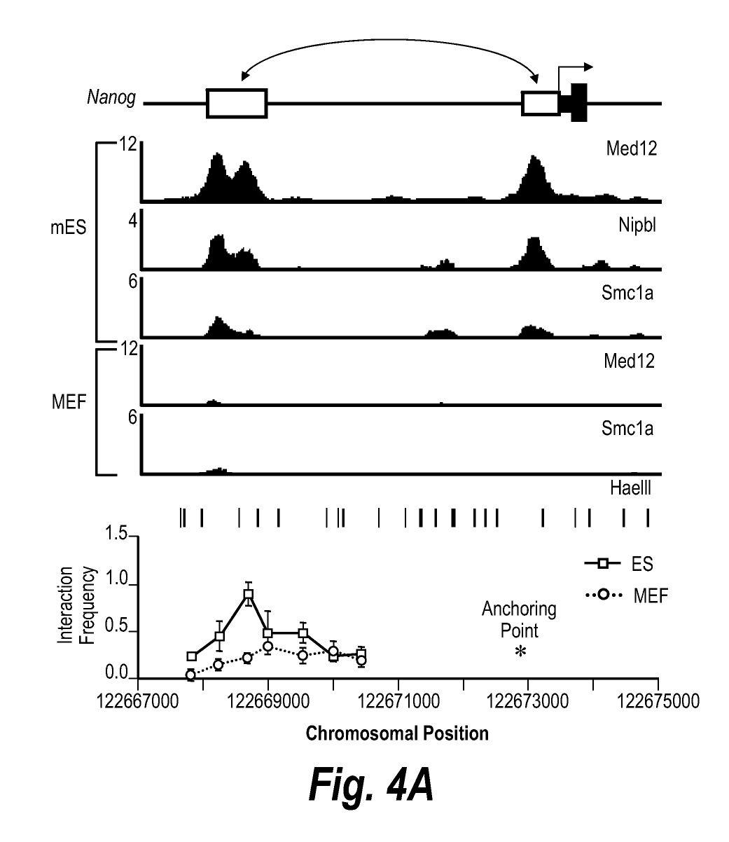

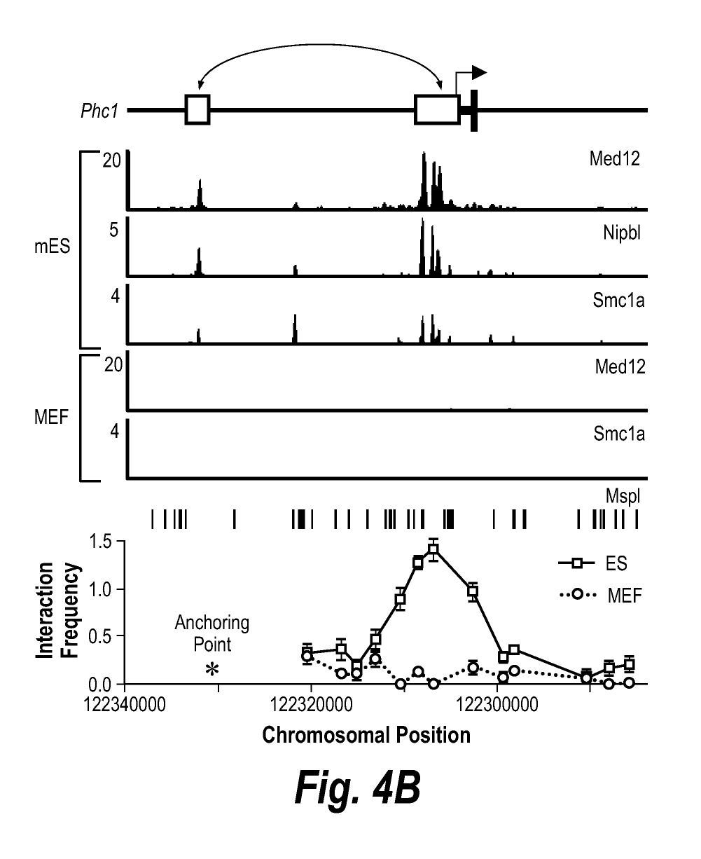

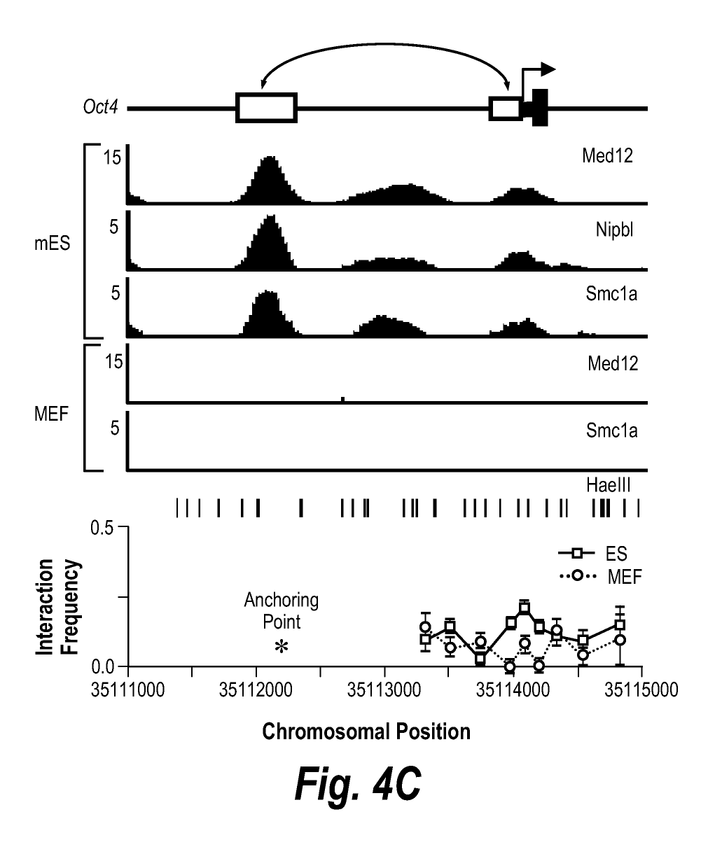

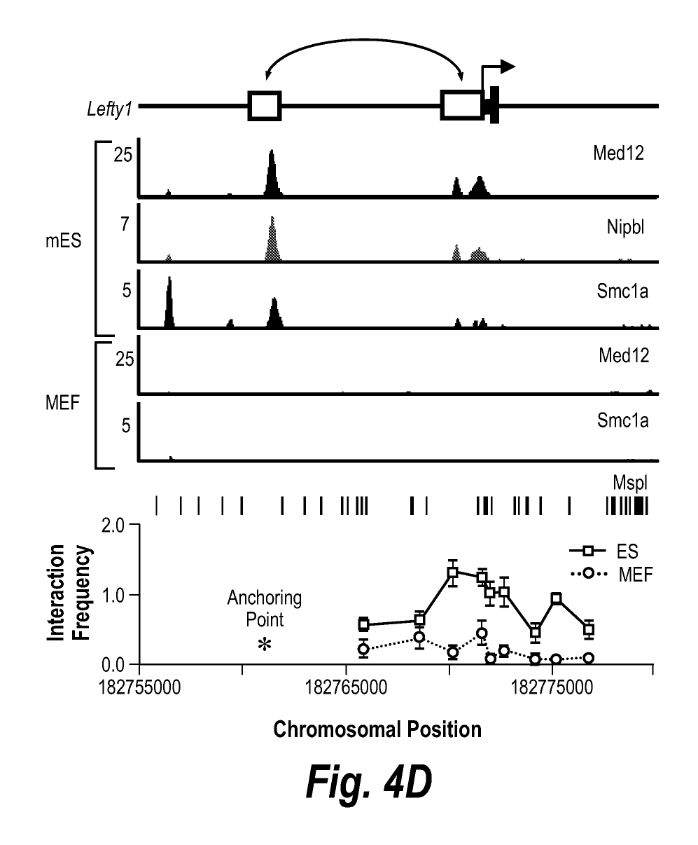

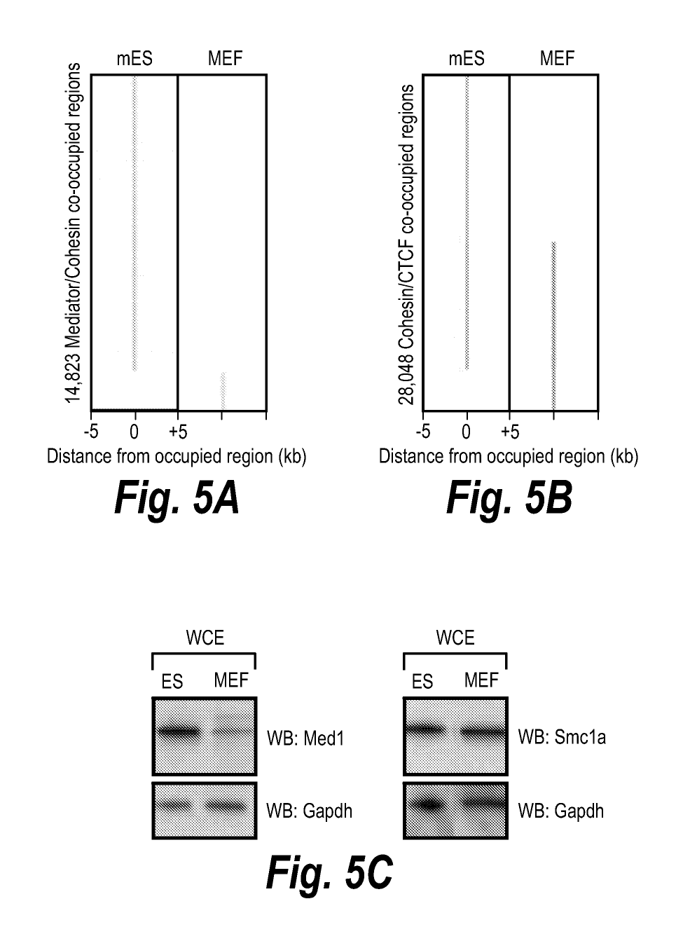

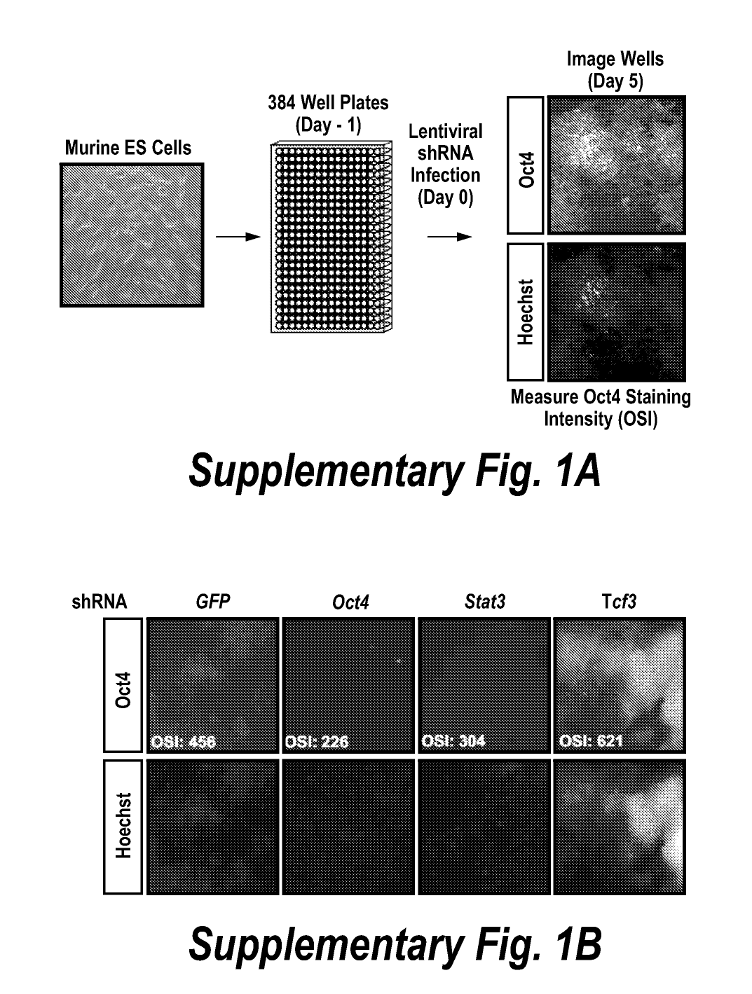

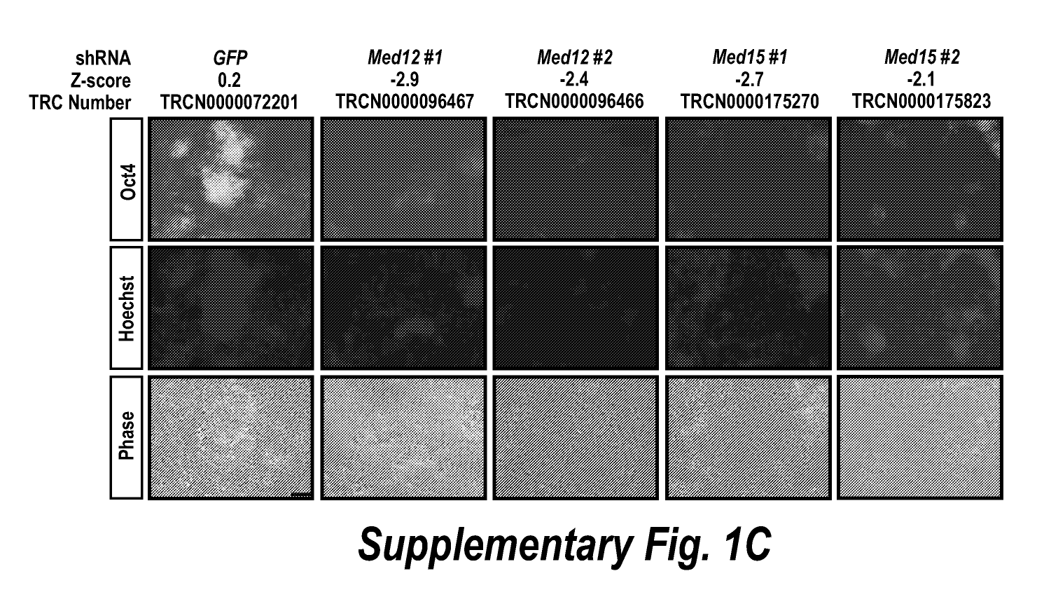

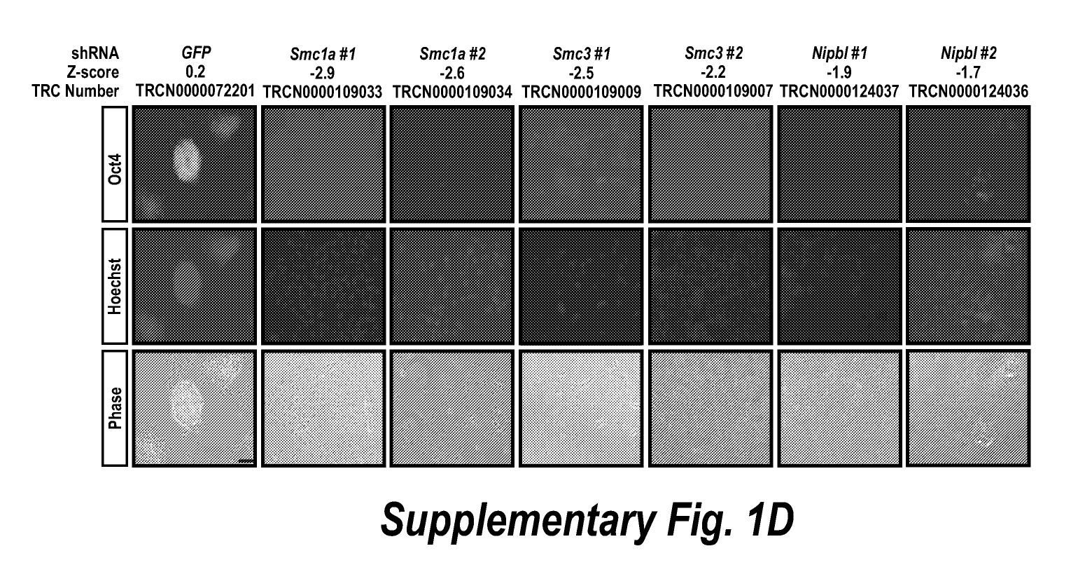

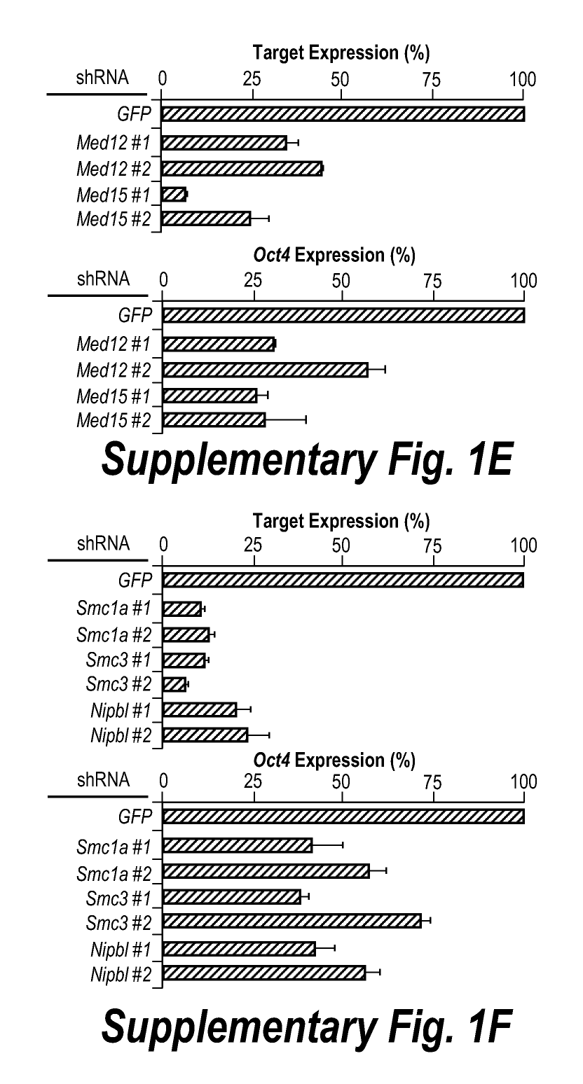

Supplementary FIG. 1: Screening protocol and validation of mediator and cohesin shRNAs. a, Outline of the screening protocol. Murine embryonic stem cells were seeded without a MEF feeder layer into 384-well plates. The following day cells were infected with individual lentiviral shRNAs targeting chromatin regulators and transcription factors. Infections were done in quadruplicate (chromatin regulator set) or duplicate (transcription factor set) on separate plates (Supplementary Table 1). Five days post-infection cells were fixed and stained with Hoechst and for Oct4. Cells were identified based on the Hoechst staining and the average Oct4 staining intensity was quantified using Cellomics software. b, Representative images from control wells on a 384-well plate infected with shRNAs targeting positive regulators of pluripotency (Oct4 and Stat3) and a negative regulator of pluripotency (Tcf3)¹⁻⁵. OSI indicates the average Oct4 staining intensity of the cells in the well, c, d, Multiple shRNAs targeting mediator (c) and cohesin (d) components reduce Oct4 protein levels and result in changes in colony morphology. Murine ES cells were infected with the indicated shRNA and stained with Hoechst and for Oct4. Scale bar=100 μM. e, f, Effect of multiple mediator and cohesin shRNAs on transcript levels for Med12, Med15, Smc1a, Smc3, Nipb1 and Oct4. Murine ES cells were infected with the indicated shRNA and transcript levels were evaluated by real-time qPCR. The error bars represent the standard deviation of the average of 3 independent PCR reactions.

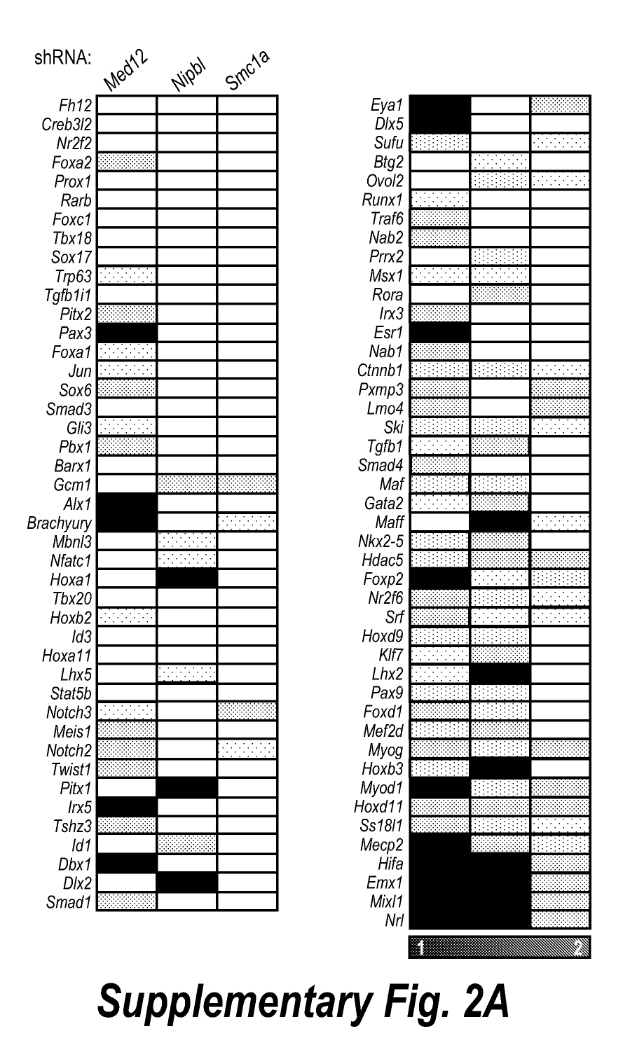

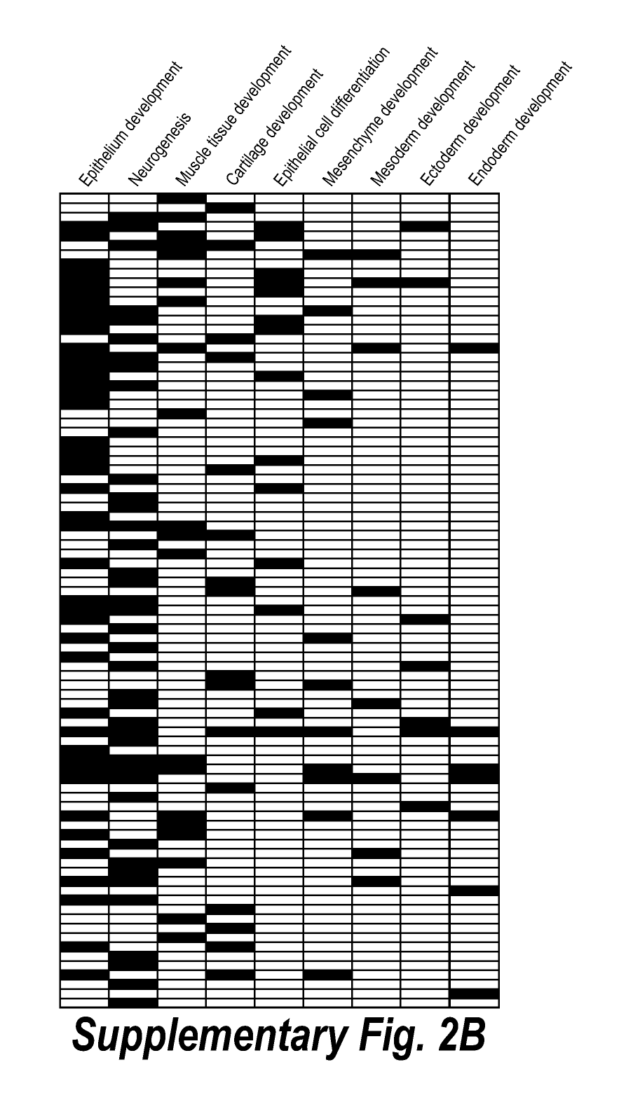

Supplementary FIG. 2: Annotation of upregulated transcription factor genes in the Med12, Nipb1, and Smc1a knockdown expression datasets. a, Heat map demonstrating that the decreased expression of Med12, Nipb1, and Smc1a result in the upregulation of a similar set of developmental transcription factor genes. Genes that are displayed are upregulated following Med12, Nipb1, and Smc1a knockdowns, and were annotated in at least one of the Gene Ontology categories shown in b. Genes were rank ordered based on the mean expression changes for the Med12 and Nipb1 knockdowns. This was done because mediator-Nipb1 occupy one set of sites whereas cohesin can occupy two sets of sites, cohesin-CTCF or cohesin-mediator-Nipb1. Expression data was generated from ES cells that were infected with GFP control, Med12, Nipb1, or Smc1a shRNAs. Five days post-infection, gene expression levels relative to the control GFP infection were determined with Agilent whole genome expression arrays. A relative signal scale is shown at the bottom of the panel. b, The decreased expression of Med12, Nipb1, and Smc1a result in the upregulation of transcription factor genes associated with developmental processes. Developmental categories from Gene Ontology (GO) are indicated at the top of the display. The annotation of a gene in the GO category is denoted by a blue box.

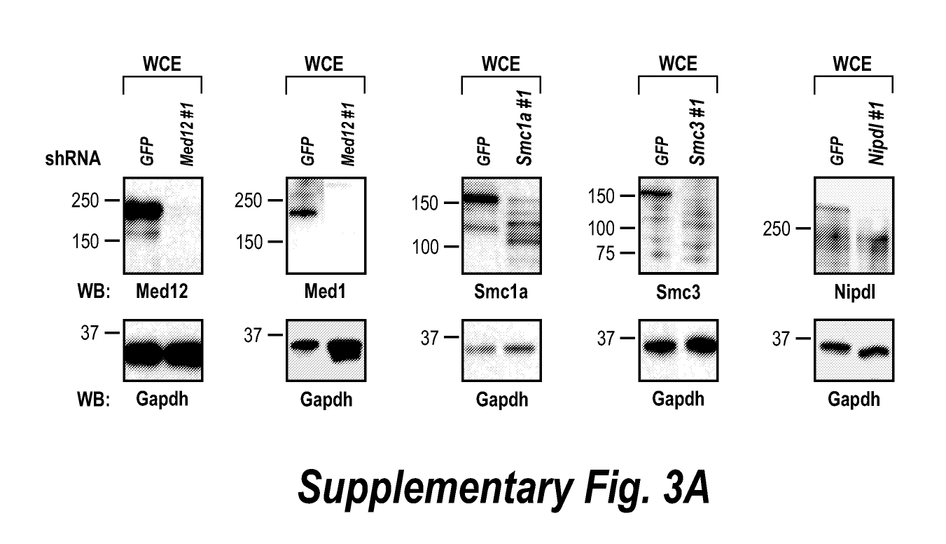

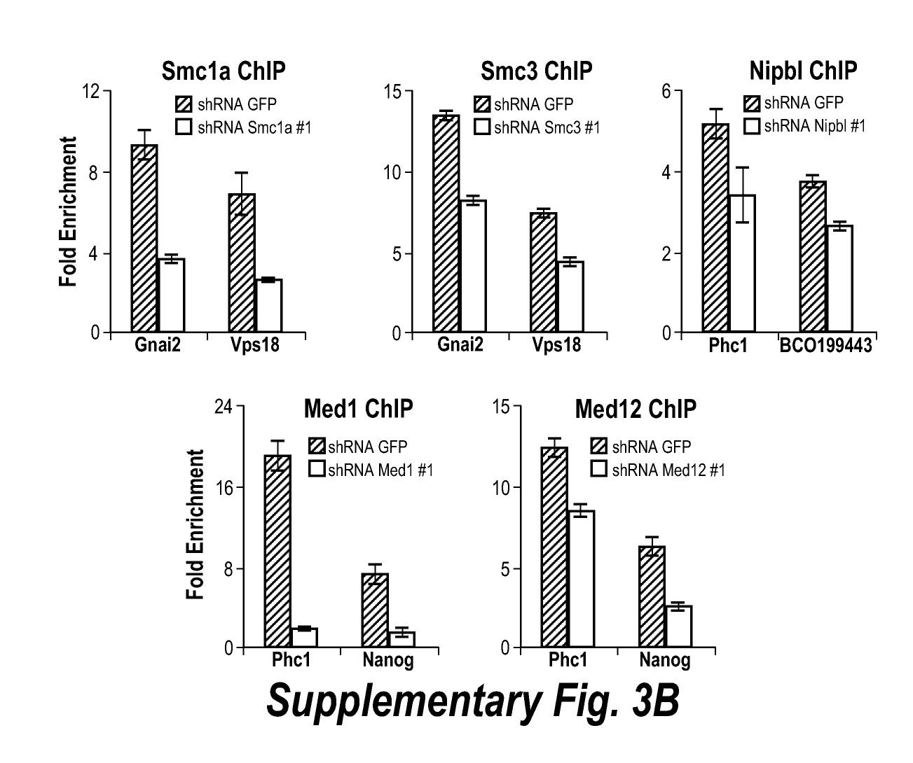

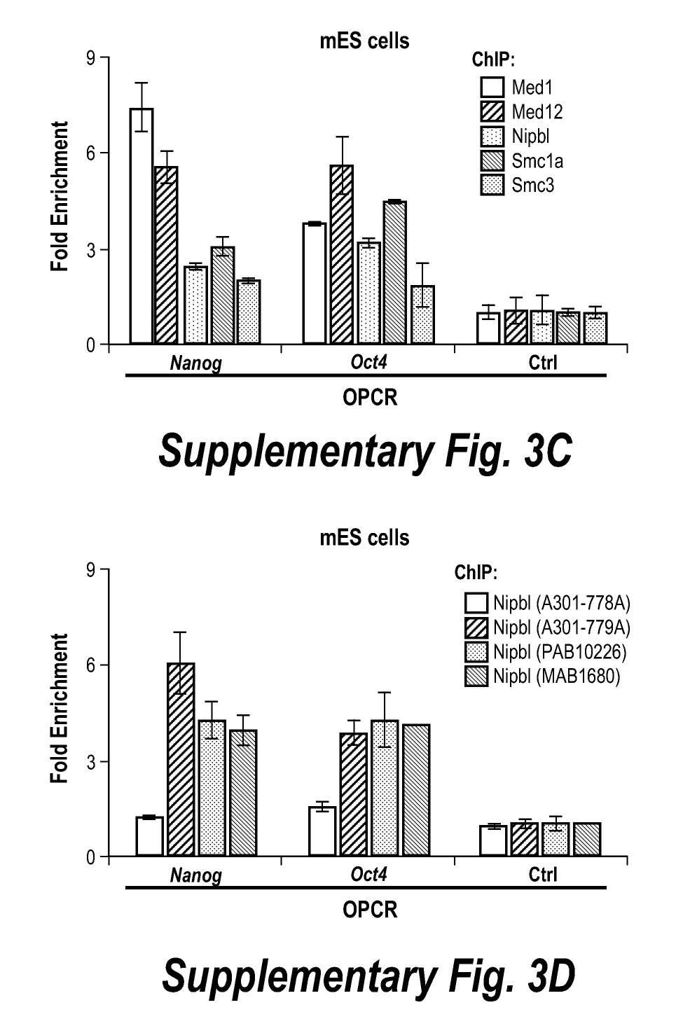

Supplementary FIG. 3: Validation of mediator, cohesin and nipb1 antibodies used for ChIP-Seq. a, Antibodies against Med12, Med1, Smc1a, Smc3 and Nipb1 are specific and shRNAs targeting Med12, Med1, Smc1a, Smc3 and Nipb1 result in reduced levels of the target protein. Murine ES cells were infected with the indicated shRNA and protein levels were determined by western blot analysis. b, Gene specific ChIPs demonstrating that a reduction in Smc1a, Smc3, Nipb1, Med1 and Med12 protein levels by shRNA result in a decreased ChIP signal at the indicated gene. Murine ES cells were infected with the indicated shRNA; gene specific ChIP experiments were performed and analyzed by real-time qPCR. Fold enrichment is relative to a negative control region. The error bars represent the standard deviation of the average of 3 independent PCR reactions. c, Gene specific ChIPs verifying that mediator, cohesin and Nipb1 occupy the promoter regions of Oct4 and Nanog in ES cells. Fold enrichment is relative to a negative control region. The error bars represent the standard deviation of the average of 3 independent experiments. d, Gene specific ChIPs indicating that the Nipb1 antibodies PAB10226 and MAB1680 also enrich for Nanog and Oct4 promoter occupied Nipb1 to similar levels as the A301-779A antibody utilized to generate the ChIP-Seq dataset. Fold enrichment is relative to a negative control region. The error bars represent the standard deviation of the average of 3 independent experiments.

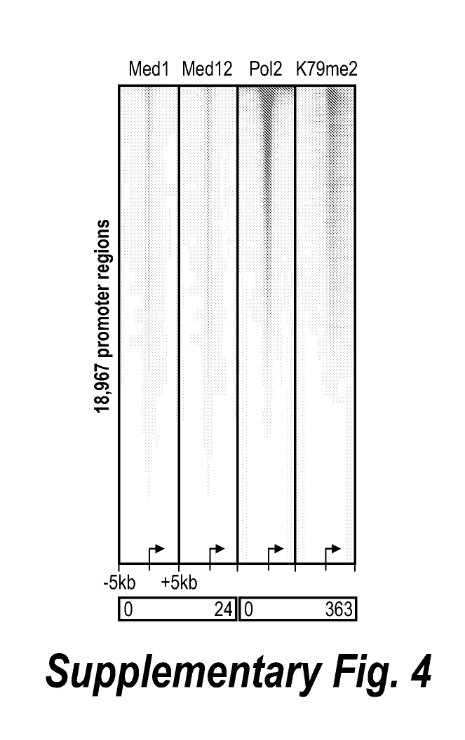

Supplementary FIG. 4: Mediator occupies the promoters of activelytranscribed genes. Density map of ChIP-Seq results for mediator (Med1, Med12), RNA polymerase II (Pol2) and di-methylated histone H3 lysine 79 (K79me2) demonstrates mediator occupancy at genes that are actively transcribed in ES cells. Normalized read counts are shown for 10 kb surrounding 18,967 Refseq promoters (from −5 kb to +5 kb) sorted by maximum level of Pol2 enrichment. A relative signal scale (reads/million) and the position of the transcription start site are shown at the bottom of the panel.

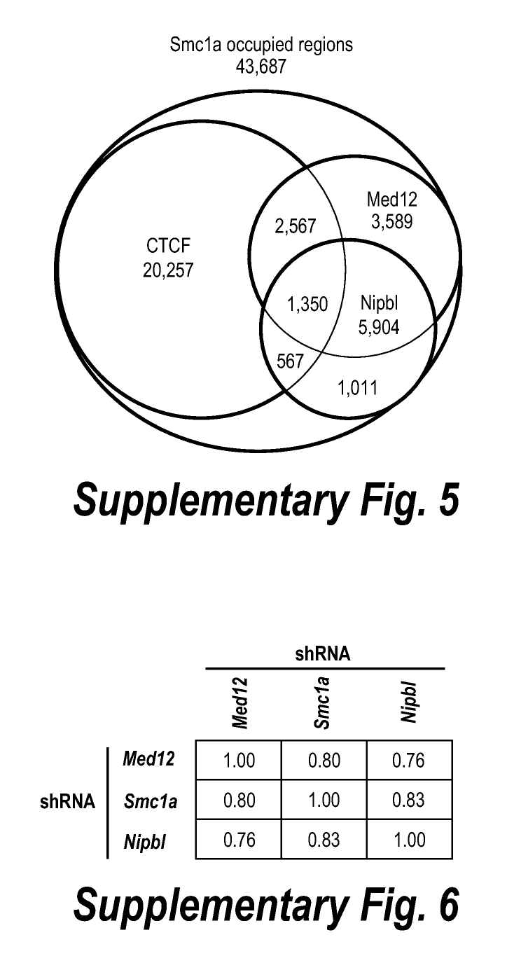

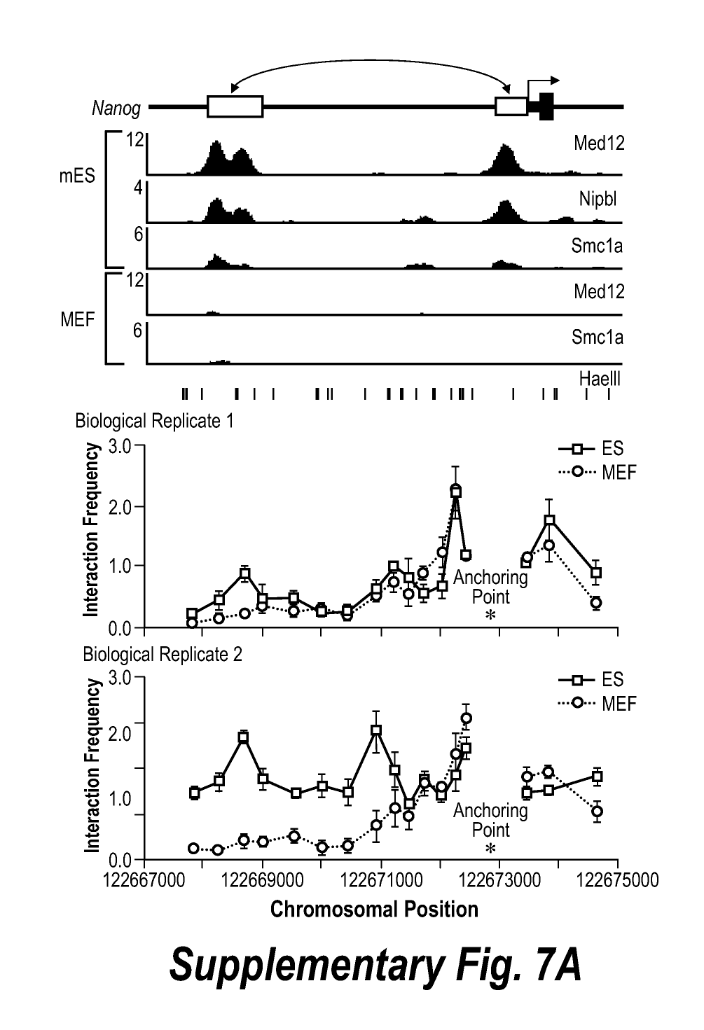

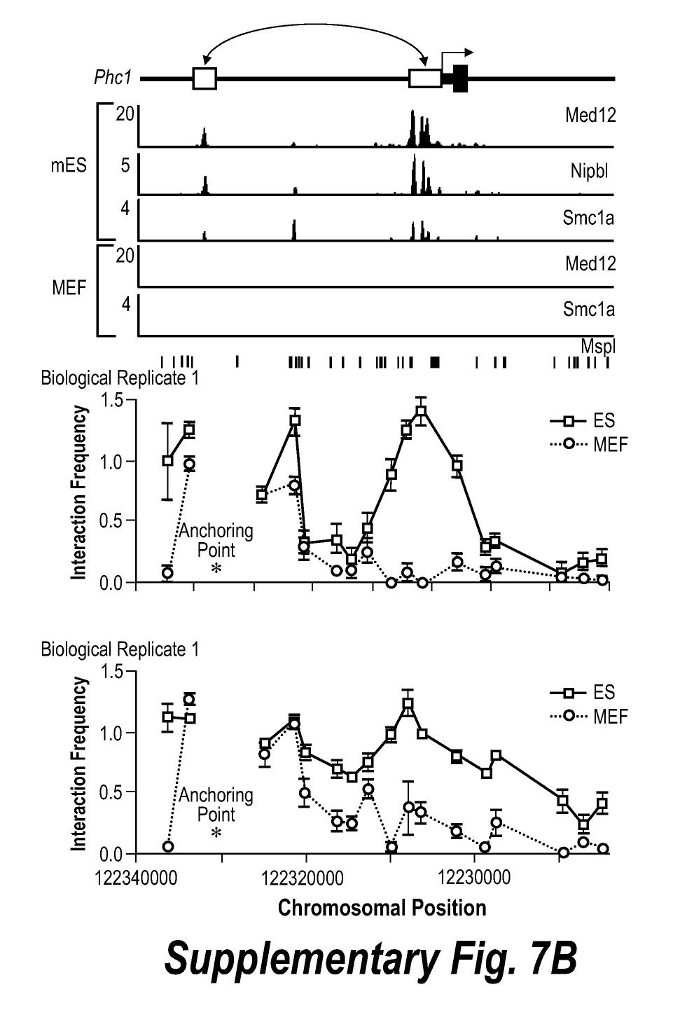

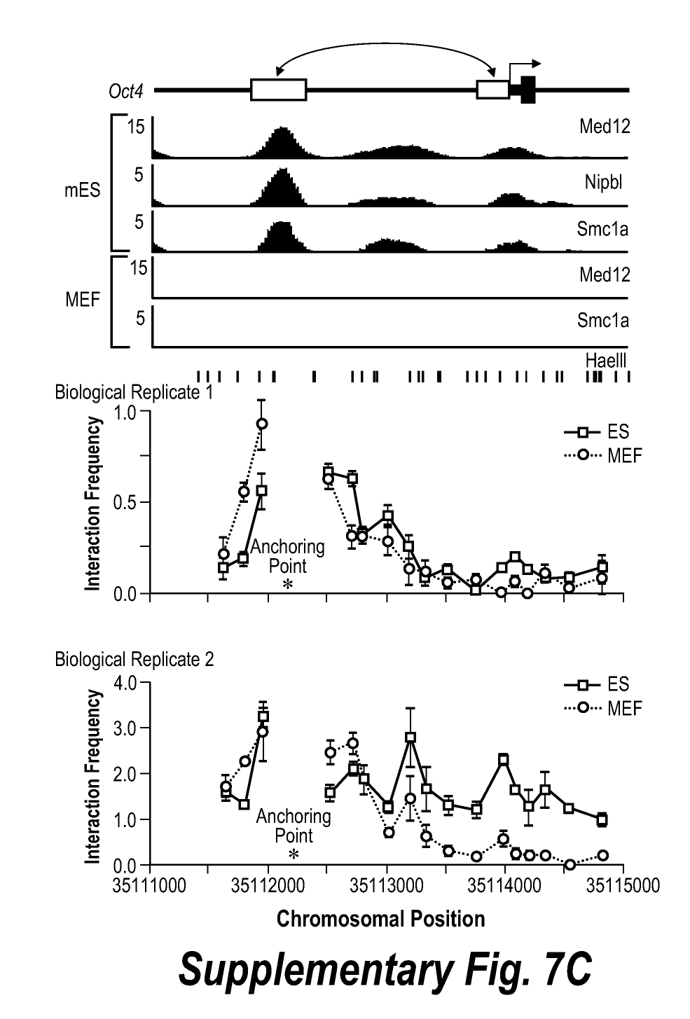

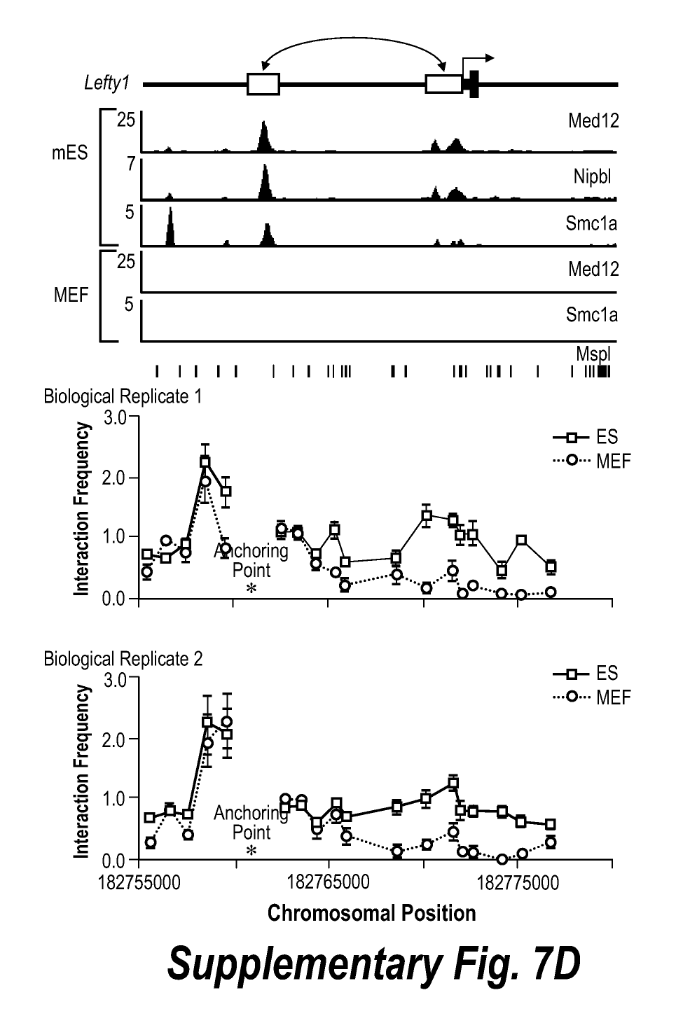

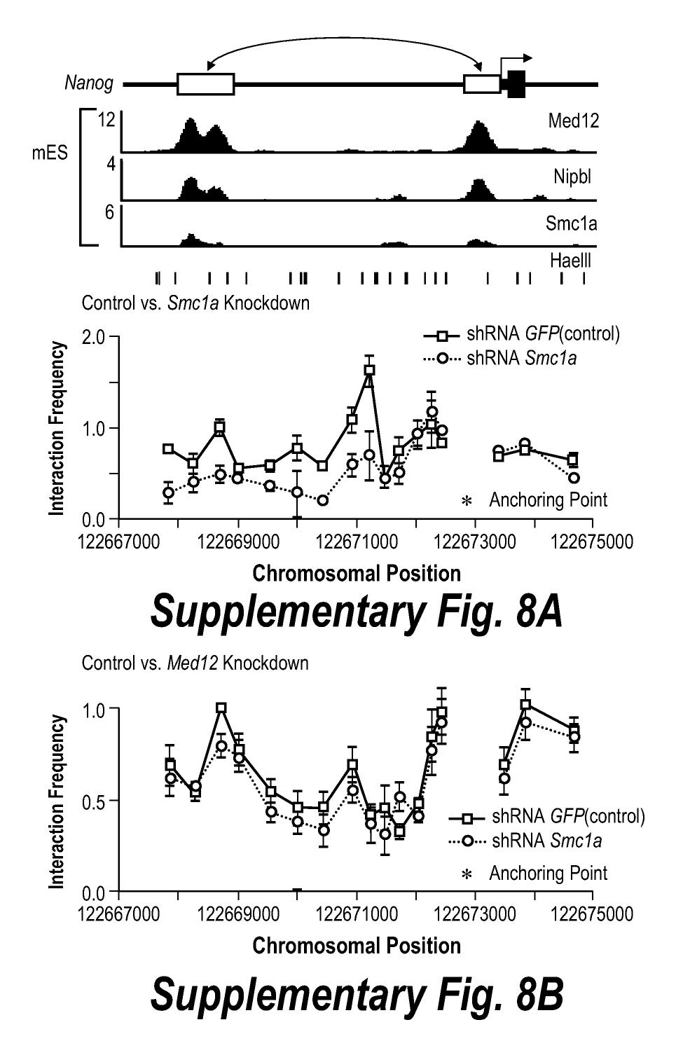

Supplementary FIG. 5: Nipb1 occupies regions co-occupied by mediator and cohesin. Venn diagram demonstrating the overlap of high confidence (Pval<10⁻⁹) CTCF, mediator (Med12) and Nipb1 occupied sites with cohesin (Smc1a). The overlap of Smc1a, Med12 and Nipb1 sites is highly significant (Pval<10⁻³⁰⁰), whereas the overlap of Smc1a, CTCF and Nipb1 is no greater than expected by chance (P-val=1).

Supplementary FIG. 6: Mediator, cohesin and Nipb1 knockdown expression datasets are similar. Pearson correlations indicate that the expression changes are similar at genes co-occupied by mediator (Med12), cohesin (Smc1a) and Nipb1 in response to a Med12, Smc1a or Nipb1 knockdown. Genes used for the analysis have evidence of a co-occupied Smc1a-Med12Nipb1 region within the gene body or within 10 kb upstream of the transcriptional start site, evidence of Pol2 occupancy within the gene body and significant (P-val<0.01) expression changes for a Smc1a, Med12 and Nipb1 knockdown in independent experiments. Gene expression levels relative to the control GFP infection were determined with Agilent whole genome expression arrays.

Supplementary FIG. 7: Mediator and cohesin binding profiles predict enhancer-promoter looping events. a-d, A looping event between the upstream enhancer and the core promoter of Nanog, Phc1, Oct4 (Pou5f1) and Lefty1 was detected by Chromosome Conformation Capture (3C) in ES cells, but not in MEFs. Biological replicates are shown for each locus. ES cell and MEF crosslinked chromatin was digested by the indicated restriction enzyme and religated under conditions that favor intramolecular ligation events. The interaction frequency between the anchoring point and distal fragments was determined by PCR and normalized to BAC templates and control regions. The restriction enzyme sites are indicated above the 3C graph. The error bars represent the standard error of the average of 3 independent PCR reactions. The genomic coordinates are NCBI build 36/mm8. The ChIP-Seq binding profiles for Med12, Nipb1 and Smc1a are shown in reads/million with the base of the y-axis set to 0.5 reads/million.

Supplementary FIG. 8: Enhancer-promoter looping at Nanog decreases with a mediator or cohesin knockdown. Chromosome Conformation Capture (3C) data demonstrating that the interaction frequency between the promoter and enhancer of Nanog decreases for a cohesin (Smc1a) or a mediator (Med12) knockdown. ES cells were infected with a control shRNA (GFP) or shRNAs targeting Smc1a or Med12. Crosslinked chromatin was digested by the HaeIII restriction enzyme and religated under conditions that favor intramolecular ligation events. The interaction frequency between the anchoring point and distal fragments was determined by PCR and normalized to BAC templates and control regions. For both graphs the interaction frequency between primer Nanog 4 (within the enhancer, Supplementary Table 7) and primer Nanog 20 (anchoring primer, Supplementary Table 7) was normalized to 1 for the control shRNA (GFP) infected cells. All other interaction frequencies were scaled accordingly. The restriction enzyme sites are indicated above the 3C graph. The error bars represent the standard error of the average of 3 independent PCR reactions. The genomic coordinates are NCBI build 36/mm8. The ChIP-Seq binding profiles for Med12, Nipb1 and Smc1a are shown in reads/million with the base of the y-axis set to 0.5 reads/million.

### DETAILED DESCRIPTION

The present invention relates at least in part to the recognition that Mediator and Cohesin physically and functionally connect the enhancers and core promoters of active genes. As described herein, it has been discovered that Mediator, a multi-subunit transcriptional coactivator, forms a complex with Cohesin, which can form rings that connect two DNA segments. The Cohesin loading factor Nipb1 is associated with such complexes, providing a means to load Cohesin at promoters. DNA looping is observed between the enhancers and promoters occupied by Mediator and Cohesin. Mediator and Cohesin co-occupy different promoters in different cells, thus generating cell-type-specific DNA loops linked to the gene expression program of cells.

The invention provides compositions and methods relating to the Mediator-Cohesin interaction. In some aspects, the compositions and/or methods are of use for diagnostic purposes, e.g., to diagnose or aid in the diagnosis of a disorder, e.g., a disorder associated with mutation(s) in one or more Mediator or Cohesin components. In some aspects, the compositions and/or methods are useful for research purposes, e.g., to elucidate mechanisms of transcriptional regulation, e.g., cell-type specific transcriptional regulation. Elucidation of such mechanisms is of use, among other things, in the development and characterization of compounds for treating disorders and/or in the development of cell-based therapies. In some aspects, the compositions and/or methods are of use in the identification of compounds that modulate cell state, e.g., for therapeutic or research purposes. In some aspects, the invention provides methods comprising detecting and, optionally, quantifying, an interaction, e.g., a physical interaction between one or more Cohesin components and one or more Mediator components. In some embodiments, a method comprises detecting and, optionally, quantifying, an interaction, e.g., a physical interaction, between a Cohesin complex and a Mediator complex.

In some embodiments, the invention relates to modulating function of a Cohesin-Mediator complex, e.g., for experimental or therapeutic purposes. The invention provides compositions and methods relating to modulating function of a Cohesin-Mediator complex. The invention encompasses the recognition that modulating function of a Cohesin-Mediator complex provides a means of modifying, e.g., controlling or regulating, cell state. Since Cohesin-Mediator binds to cell type specific genes and, e.g., regulates their activity (e.g., transcription), modulating a Cohesin-Mediator function will in turn modify cell state. The invention thus provides in some embodiments methods for modifying cell state, e.g., in a cell-type specific manner. In some aspects, the methods involve modulating a Cohesin-Mediator function, Cell type specific genes include, e.g., many of the genes that are responsible for establishing and/or maintaining cell state. In some embodiments, such genes include, e.g., transcription factors, co-activators, and/or chromatin modulators. Modifying cell state in a cell type specific manner can include e.g., modifying the state of one or more selected cell types while, in some embodiments, not modifying (or having a lesser effect on) cells of one or more other types. Modifying cell state in a cell type specific manner can include, e.g., modifying the state of cells that have an abnormal cell state, while, in some embodiments, not modifying (or having a lesser effect on) cells that do not exhibit the abnormal state.

In some embodiments, the invention provides a method of modifying cell state comprising modulating a function (activity) of a Cohesin-Mediator complex. In some embodiments, a function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In some embodiments, modulating the binding of a Cohesin component to a Mediator component comprises modulating the binding of a Cohesin component to a complex comprising the Mediator component. In some embodiments, modulating the binding of a Mediator component to a Cohesin component comprises modulating the binding of a Mediator component to a complex comprising the Cohesin component.

In some embodiments, the invention provides methods of modifying cell state. In some aspects, cell state reflects the fact that cells of a particular type can exhibit variability with regard to one or more features and/or can exist in a variety of different conditions, while retaining the features of their particular cell type and not gaining features that would cause them to be classified as a different cell type. The different states or conditions in which a cell can exist may be characteristic of a particular cell type (e.g., they may involve properties or characteristics exhibited only by that cell type and/or involve functions performed only or primarily by that cell type) or may occur in multiple different cell types. Sometimes a cell state reflects the capability of a cell to respond to a particular stimulus or environmental condition (e.g., whether or not the cell will respond, or the type of response that will be elicited) or is a condition of the cell brought about by a stimulus or environmental condition. Cells in different cell states may be distinguished from one another in a variety of ways. For example, they may express, produce, or secrete one or more different genes, proteins, or other molecules (“markers”), exhibit differences in protein modifications such as phosphorylation, acetylation, etc., or may exhibit differences in appearance. Thus a cell state may be a condition of the cell in which the cell expresses, produces, or secretes one or more markers, exhibits particular protein modification(s), has a particular appearance, and/or will or will not exhibit one or more biological response(s) to a stimulus or environmental condition. Markers can be assessed using methods well known in the art, e.g., gene expression can be assessed at the mRNA level using Northern blots, cDNA or oligonucleotide microarrays, or sequencing (e.g., RNA-Seq), or at the level of protein expression using protein microarrays, Western blots, flow cytometry, immunohistochemistry, etc. Modifications can be assessed, e.g., using antibodies that are specific for a particular modified form of a protein, e.g., phospho-specific antibodies, or mass spectrometry.

Another example of cell state is “activated” state as compared with “resting” or “non-activated” state. Many cell types in the body have the capacity to respond to a stimulus by modifying their state to an activated state. The particular alterations in state may differ depending on the cell type and/or the particular stimulus. A stimulus could be any biological, chemical, or physical agent to which a cell may be exposed. A stimulus could originate outside an organism (e.g., a pathogen such as virus, bacteria, or fungi (or a component or product thereof such as a protein, carbohydrate, or nucleic acid, cell wall constituent such as bacterial lipopolysaccharide, etc) or may be internally generated (e.g., a cytokine, chemokine, growth factor, or hormone produced by other cells in the body or by the cell itself). For example, stimuli can include interleukins, interferons, or TNF alpha. Immune system cells, for example, can become activated upon encountering foreign (or in some instances host cell) molecules. Cells of the adaptive immune system can become activated upon encountering a cognate antigen (e.g., containing an epitope specifically recognized by the cell's T cell or B cell receptor) and, optionally, appropriate co-stimulating signals. Activation can result in changes in gene expression, production and/or secretion of molecules (e.g., cytokines, inflammatory mediators), and a variety of other changes that, for example, aid in defense against pathogens but can, e.g., if excessive, prolonged, or directed against host cells or host cell molecules, contribute to diseases. Fibroblasts are another cell type that can become activated in response to a variety of stimuli (e.g., injury (e.g., trauma, surgery), exposure to certain compounds including a variety of pharmacological agents, radiation, etc.) leading them, for example, to secrete extracellular matrix components. In the case of response to injury, such ECM components can contribute to wound healing. However, fibroblast activation, e.g., if prolonged, inappropriate, or excessive, can lead to a range of fibrotic conditions affecting diverse tissues and organs (e.g., heart, kidney, liver, intestine, blood vessels, skin) and/or contribute to cancer. The presence of abnormally large amounts of ECM components can result in decreased tissue and organ function, e.g., by increasing stiffness and/or disrupting normal structure and connectivity.

Another example of cell state reflects the condition of cell (e.g., a muscle cell or adipose cell) as either sensitive or resistant to insulin. Insulin resistant cells exhibit decreased respose to circulating insulin; for example insulin-resistant skeletal muscle cells exhibit markedly reduced insulin-stimulated glucose uptake and a variety of other metabolic abnormalities that distinguish these cells from cells with normal insulin sensitivity.

As used herein, a “cell state associated gene” is a gene the expression of which is associated with or characteristic of a cell state of interest (and is often not associated with or is significantly lower in many or most other cell states) and may at least in part be responsible for establishing and/or maintaining the cell state. For example, expression of the gene may be necessary or sufficient to cause the cell to enter or remain in a particular cell state. In some embodiments of the invention, modulating a function of a Cohesin-Mediator complex alters the expression of gene(s) whose transcription is activated by Cohesin-Mediator complex, e.g., cell type specific gene(s) or cell state associated gene(s), and thereby alters cell type or cell state. In some embodiments of the invention, modulating a function (activity) of a Cohesin-Mediator complex alters occupancy of a cell state associated gene by Cohesin-Mediator complex. According to certain aspects of the invention, a Cohesin-Mediator complex occupies cell type specific genes in tumor cells (or other cells having an abnormal state associated with a disorder). For example, Cohesin-Mediator complex can occupy genes that are selectively expressed in tumor cells (or in cancer-associated cells such as stromal cells in a tumor), e.g., genes that drive aberrant proliferation, migration, metastasis, or other properties associated with tumors. The invention provides means to selectively modify cell type specific phenotypes, e.g., phenotype(s) of a tumor cell or other cell having an abnormal state associated with a disorder. In some aspects, modulating a Cohesin-Mediator function shifts a cell from an “abnormal” state towards a more “normal” state. In some embodiments, modulating a Cohesin-Mediator function shifts a cell from a “disease-associated” state towards a state that is not associated with disease. A “disease-associated state” is a state that is typically found in subjects suffering from a disease (and usually not found in subjects not suffering from the disease) and/or a state in which the cell is abnormal, unhealthy, or contributing to a disease. In some aspects, modulating a Cohesin-Mediator function has a cell type specific effect, e.g., it modifies the state of cells of a certain type but not one or more other types.

In some embodiments, modulating a function (activity) of a Cohesin-Mediator complex is of use to treat, e.g., a metabolic, neurodegenerative, inflammatory, auto-immune, proliferative, infectious, cardiovascular, musculoskeletal, or other disease. It will be understood that diseases can involve multiple pathologic processes and mechanisms and/or affect multiple body systems. Discussion herein of a particular disease in the context of a particular pathologic process, mechanism, cell state, cell type, or affected organ, tissue, or system, should not be considered limiting. For example, a number of different tumors (e.g., hematologic neoplasms such as leukemias) arise from undifferentiated progenitor cells and/or are composed largely of undifferentiated or poorly differentiated cells that retain few if any distinctive features characteristic of differentiated cell types. These tumors, which are sometimes termed undifferentiated or anaplastic tumors, may be particularly aggressive and/or difficult to treat. In some embodiments of the invention, a method of the invention is used to modify such cells to a more differentiated state, which may be less highly proliferative and/or more amenable to a variety of therapies, e.g., chemotherapeutic agents. In another embodiment, an inventive method is used to treat insulin resistance which occurs, for example, in individuals suffering from type II diabetes and pre-diabetic individuals. It would be beneficial to modify the state of insulin-resistant cells towards a more insulin-sensitive state, e.g., for purposes of treating individuals who are developing or have developed insulin resistance. In another embodiment, an inventive method is used to treat obesity.

Many inflammatory and/or autoimmune conditions may occur at least in part as a result of excessive and/or inappropriate activation of immune system cells. Autoimmune diseases include, e.g., Graves disease, Hashimoto's thyroiditis, myasthenia gravis, rheumatoid arthritis, sarcoidosis, Sjögren's syndrome, scleroderma, ankylosing spondylitis, type I diabetes, vasculitis, and lupus erythematosus. Furthermore, immune-mediated rejection is a significant risk in organ and tissue transplantation. Inflammation plays a role in a large number of diseases and conditions. Inflammation can be acute (and may be recurrent) or chronic. In general, inflammation can affect almost any organ, tissue, or body system. For example, inflammation can affect the cardiovascular system (e.g., heart), musculoskeletal system, respiratory system (e.g., bronchi, lungs), renal system, (e.g., kidneys), eyes, nervous system, gastrointestinal system (e.g., colon), integumentary system (e.g., skin), musculoskeletal system (e.g., joints, muscles), resulting in a wide variety of conditions and diseases. Chronic inflammation is increasingly recognized as an important factor contributing to atherosclerosis and degenerative diseases of many types. Inflammation influences the microenvironment around tumours and contributes, e.g., to tumor cell proliferation, survival and migration. Furthermore, chronic inflammation can eventually lead to fibrosis.

Exemplary inflammatory diseases include, e.g., adult respiratory distress syndrome (ARDS), atherosclerosis (e.g., coronary artery disease, cerebrovascular disease), allergies, asthma, cancer, demyleinating diseases, dermatomyositis, inflammatory bowel disease (e.g., Crohn's disease, ulcerative colitis), inflammatory myopathies, multiple sclerosis, glomerulonephritis, psoriasis, pancreatitis, rheumatoid arthritis, sepsis, vasculitis (including phlebitis and arteritis, e.g., polyarteritis nodosa, Wegener's granulomatosis, Buerger's disease, Takayasu's arteritis, etc.). In some embodiments, a method of the invention is used to modify immune cell state to reduce activation of immune system cells involved in such conditions and/or render immune system cells tolerant to one or more antigens. In one embodiment, dendritic cell state is altered. Promoting immune system activation using a method of the invention (e.g., in individuals who have immunodeficiencies or have been treated with drugs that deplete or damage immune system cells), potentially for limited periods of time, may be of benefit in the treatment of infectious diseases.

In other embodiments, activated fibroblasts are modified to a less activated cell state to reduce or inhibit fibrotic conditions or treat cancer.

Post-surgical adhesions can be a complication of, e.g., abdominal, gynecologic, orthopedic, and cardiothoracic surgeries. Adhesions are associated with considerable morbidity and can be fatal. Development of adhesions involves inflammatory and fibrotic processes. In some embodiments, a method of the invention is used to modify state of immune system cells and/or fibroblasts to prevent or reduce adhesion formation or maintenance.

In other embodiments, modifying cells to a more or less differentiated state is of use to generate a population of cells in vivo that aid in repair or regeneration of a diseased or damaged organ or tissue, or to generate a population of cells ex vivo that is then administered to a subject to aid in repair or regeneration of a diseased or damaged organ or tissue.

In some embodiments, cell type and or cell state becomes modified over the course of multiple cell cycle(s). In some embodiments, cell type and/or cell state is stably modified. In some embodiments, a modified type or state may persist for varying periods of time (e.g., days, weeks, months, or indefinitely) after the cell is no longer exposed to the agent(s) that caused the modification. In some embodiments, continued or at intermittent exposure to the agent(s) is required or helpful to maintain the modified state or type.

Cells may be in living animal, e.g., a mammal, or may be isolated cells. Isolated cells may be primary cells, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In many embodiments, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc. Cells may be obtained from any organ or tissue of interest. In some embodiments, cells are obtained from skin, lung, cartilage, breast, blood, blood vessel (e.g., artery or vein), fat, pancreas, liver, muscle, gastrointestinal tract, heart, bladder, kidney, urethra, prostate gland. Cells may be maintained in cell culture following their isolation. In certain embodiments, the cells are passaged or allowed to double once or more following their isolation from the individual (e.g., between 2-5, 5-10, 10-20, 20-50, 50-100 times, or more) prior to their use in a method of the invention. They may be frozen and subsequently thawed prior to use. In some embodiments, the cells will have been passaged or permitted to double no more than 1, 2, 5, 10, 20, or 50 times following their isolation from the individual prior to their use in a method of the invention. Cells may be genetically modified or not genetically modified in various embodiments of the invention. Cells may be obtained from normal or diseased tissue. In some embodiments, cells are obtained from a donor, and their state or type is modified ex vivo using a method of the invention. The modified cells are administered to a recipient, e.g., for cell therapy purposes. In some embodiments, the cells are obtained from the individual to whom they are subsequently administered.

A population of isolated cells in any embodiment of the invention may be composed mainly or essentially entirely of a particular cell type or of cells in a particular state. In some embodiments, an isolated population of cells consists of at least 30%, 40%, 50%, 60%, 70%, 80%, 90%, 95%, 96%, 97%, 98%, 99%, or 100% cells of a particular type or state (i.e., the population is at least 30%, 40%, 50%, 60%, 70%, 80%, 90%, 95%, 96%, 97%, 98%, 99%, or 100% pure), e.g., as determined by expression of one or more markers or any other suitable method.

In some embodiments, the invention provides a method of modifying cell type comprising modulating a function (activity) of a Cohesin-Mediator complex. In some embodiments, a function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway. In various embodiments, a cell type can be any of the distinct forms of cell found in the body of a normal, healthy adult vertebrate, e.g., a mammal (e.g., a mouse of human) or avian. Typically, different cell types are distinguishable from each other based on one or more structural characteristics, functional characteristics, gene expression profile, proteome, secreted molecules, cell surface marker (and/or other marker) expression (e.g., CD molecules), or a combination of any of these. In general, members of a particular cell type display at least one characteristic not displayed by cells of other types or display a combination of characteristics that is distinct from the combination of characteristics found in other cell types. Members of the cell type are typically more similar to each other than they are to cells of different cell types. See, e.g., Young, B., et al., Wheater's Functional Histology: A Text and Colour Atlas, 5th ed. Churchill Livingstone, 2006, or Alberts, B., et al, Molecular Biology of the Cell, 4th ed, (2002) or 5th edition (2007), Garland Science, Taylor & Francis Group, for exemplary cell types and characteristic features thereof. In some embodiments, a cell is of a cell type that is typically classified as a component of one of the four basic tissue types, i.e., connective, epithelial, muscle, and nervous tissue. In some embodiments of the invention, a cell is a connective tissue cell. Connective tissue cells include storage cells (e.g., brown or white adipose cells, liver lipocytes), extracellular matrix (ECM)-secreting cells (e.g., fibroblasts, chondrocytes, osteoblasts), and blood/immune system cells such as lymphocytes (e.g., T lymphocytes, B lymphocytes, or plasma cells), granulocytes (e.g., basophils, eosinophils, neutrophils), and monocytes. In some embodiments of the invention, a cell is an epithelial cell. Epithelial cell types include, e.g., gland cells specialized for secretion such as exocrine and endocrine glandular epithelial, and surface epithelial cells such as keratinizing and non-keratinizing surface epithelial cells. Nervous tissue cells include glia cells and neurons of the central or peripheral nervous system. Muscle tissue cells include skeletal, cardiac, and smooth muscle cells. Many of these cell types can be further categorized. For example, T lymphocytes include helper, regulatory, and cytotoxic T cells. Cell types can be classified based on the germ layer from which they originate. In some embodiments, a cell is of endodermal origin. In some embodiments, a cell is of mesodermal origin. In some embodiments, a cell is of ectodermal origin. Cell types can be classified based on the germ layer from which they originate. In some embodiments, a cell is of endodermal origin. In some embodiments, a cell is of mesodermal origin. In some embodiments, a cell is of ectodermal origin. In some embodiments, a cell type is a stem cell, e.g., an adult stem cell. Exemplary adult stem cells include hematopoietic stem cells, neural stem cells, and mesenchymal stem cells. In some embodiments, a cell type is a mature, differentiated cell type. In some embodiments a cell is an adipocyte (e.g., white fat cell or brown fat cell), cardiac myocyte, chondrocyte, endothelial cell, exocrine gland cell, fibroblast, hair follicle cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell.

In some embodiments, the methods and compounds herein are of use to reprogram a somatic cell, e.g., to a pluripotent state. In some embodiments the methods and compounds are of use to reprogram a somatic cell of a first cell type into a different cell type. In some embodiments, the methods and compounds herein are of use to differentiate a pluripotent cell to a desired cell type.

In some embodiments, modulating a function of a Cohesin-Mediator complex comprises disrupting a Cohesin-Mediator function. In some embodiments, disrupting a Cohesin-Mediator function reduces the expression of cell type specific gene(s) or cell state associated gene(s). In some embodiments, reduced expression of a cell type specific gene or cell state associated gene facilitates modifying the cell type or cell state to a different cell type or cell state. Modifying the cell type or cell state may be accomplished by, for example, contacting the cell with compound(s) (e.g., small molecules, proteins, siRNAs or other nucleic acids) or cells or otherwise changing its environment (e.g., changing the pit, media components such as nutrient(s), growth substrate, or proximity to cells of the same or different types). In some embodiments, the disruption in Cohesin-Mediator function is transient, so that once a cell type or state is modified at least in part, Cohesin-Mediator function is restored to a nondisrupted condition, in which it activates transcription of genes specific for or associated with the modified cell type or cell state. In some embodiments, Cohesin-Mediator function is disrupted using an siRNA, shRNA, or antisense oligonucleotide that inhibit expression of a gene encoding a Cohesin or Mediator component. In some embodiments, Cohesin-Mediator function is disrupted using an aptamer that binds to a Cohesin or Mediator component or using a dominant negative version of a Cohesin or Mediator component.

A cell type specific gene is typically expressed selectively in one or a small number of cells types relative to expression in many or most other cell types. One of skill in the art will be aware of numerous genes that are considered cell type specific. A cell type specific gene need not be expressed only in a single cell type but may be expressed in one or several, e.g., up to about 5, or about 10 different cell types out of the approximately 200 commonly recognized (e.g., in standard histology textbooks) and/or most abundant cell types in an adult vertebrate, e.g., mammal, e.g., human. In some embodiments, a cell type specific gene is one whose expression level can be used to distinguish a cell of one of the following types from cells of the other cell types: adipocyte (e.g., white fat cell or brown fat cell), cardiac myocyte, chondrocyte, endothelial cell, exocrine gland cell, fibroblast, glial cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell. In some embodiments a cell type specific gene is lineage specific, e.g., it is specific to a particular lineage (e.g., hematopoietic, neural, muscle, etc.) In some embodiments, a cell-type specific gene is a gene that is more highly expressed in a given cell type than in most (e.g., at least 80%, at least 90%) or all other cell types. Thus specificity may relate to level of expression, e.g., a gene that is widely expressed at low levels but is highly expressed in certain cell types could be considered cell type specific to those cell types in which it is highly expressed. It will be understood that expression can be normalized based on total mRNA expression (optionally including miRNA transcripts, long non-coding RNA transcripts, and/or other RNA transcripts) and/or based on expression of a housekeeping gene in a cell. In some embodiments, a gene is considered cell type specific for a particular cell type if it is expressed at levels at least 2, 5, or at least 10-fold greater in that cell than it is, on average, in at least 25%, at least 50%, at least 75%, at least 90% or more of the cell types of an adult of that species, or in a representative set of cell types. One of skill in the art will be aware of databases containing expression data for various cell types, which may be used to select cell type specific genes. In some embodiments a cell type specific gene is a transcription factor. Exemplary, non-limiting lists of cell type specific genes for ES cells and MEFs are shown in Table S11.

In some embodiments of the invention a cell type specific gene is a developmental regulator. In some embodiments a developmental regulator is a gene that falls into the Gene Ontology category “Cellular Developmental Processes”. In some embodiments, a developmentally important transcription factor is a transcription factor that falls into the Gene Ontology category “Cellular Developmental Processes”.

In some embodiments, modulating function of a Cohesin-Mediator complex is accomplished by contacting the complex with a compound. The complex can be in cells. The complex can be contacted by contacting the cells with a compound in vitro (e.g., in cell culture) or administering the compound to a subject. The compound can, e.g., be identified using an inventive method described herein. In some embodiments, e.g., where the compound is a nucleic acid or protein, contacting a cell with a compound comprises causing the cell to express the compound. For example, a cell can be stably or transiently transfected with a nucleic acid, optionally encoding a protein, or exposed to an agent, e.g., an inducing agent, that causes the cell to express a gene (which can be an endogenous gene or an exogenously introduced gene).

In some embodiments, the invention provides a method of identifying a compound that modulates a function of a Cohesin-Mediator complex comprising steps of: (a) contacting a composition comprising at least one Cohesin component and at least one Mediator component with a test compound; (b) assessing at least one function of a Cohesin-Mediator complex; (c) comparing the function measured in step (b) with a suitable reference value, wherein if the function measured in step (b) differs from the reference value, the test compound modulates function of a Cohesin-Mediator complex. In some embodiments a function is selected from the group consisting of: (a) binding of a Cohesin complex to Mediator complex or binding of a Cohesin component to a Mediator component; (b) occupancy of a cell type specific gene; (c) controlling expression or activity of a cell type specific gene; and (d) mediating response to a signal transduction pathway. It will be understood that “reference value” can comprise multiple individual values, e.g., expression levels in a gene expression profile, or multiple responses to a signal transduction pathway.

In general, a reference value of use herein could be a previously measured value selected as appropriate to the method in which it is used. One of skill in the art will be able to select an appropriate reference value. In some embodiments a previously measured value was obtained using comparable experimental conditions, except with respect to a condition whose effect is being assessed. In some embodiments a previously measured value was obtained using a cell of the same type and/or under essentially the same experimental conditions. In some embodiments a previously measured value was obtained using a cell of a different type and/or under different conditions. (Of course the reference value could be measured in parallel with or subsequent to a measurement involving a test compound.) In some embodiments a suitable reference value refers to a value that would exist in the absence of a test compound (or in the presence of a compound in an amount that has been previously shown not to affect a function or property being assessed). In some embodiments a reference value is a value obtained using Cohesin and Mediator components or complexes from “normal” cells (e.g., cells derived from a subject not suffering from a disorder of interest, e.g., a healthy subject not known to suffer from any disorder). In some embodiments a reference value is a value obtained in the presence of a compound or condition known to modulate function of a Cohesin-Mediator complex. In some embodiments a difference between a measured value and a reference value is statistically significant, e.g., has a p-value of less than 0.05, e.g., a p-value of less than 0.025 or a p-value of less than 0.01, using an appropriate statistical test.

In some embodiments, a signal transduction pathway is a signaling pathway initiated by binding of a hormone, growth factor, cytokine, or small molecule to an extracellular domain of a cell surface receptor. In some embodiments a signal transduction pathway involves a kinase, e.g., a receptor kinase, e.g., a tyrosine kinase, serine kinase, or threonine kinase. Exemplary signal transduction pathways are, e.g., the Wnt pathway, the TGF beta pathway, the Notch/Delta pathway, the Hedgehog pathway. A signal transduction pathway often relays a signal to a transcriptional modulator, e.g., a transcription factor. Exemplary transcriptional modulators associated with the Wnt and TGFbeta pathways, respectively, include e.g., TCF family members and Smad family members. In some embodiments of the invention, modulating function of a Cohesin-Mediator complex modulates expression and/or activity of a transcriptional modulator associated with a signal transduction pathway. Signal transduction pathways that, e.g., drive abnormal or undesired cell survival or proliferation are of interest in certain embodiments. In some embodiments, a response to a signal transduction pathway comprises altering, e.g., inducing or repressing, expression of certain genes, which in turn can have a variety of effects on cell state, as known in the art. Response to a signal transduction pathway can be assessed, e.g., by contacting a cell with a suitable ligand that can initiate the pathway, e.g., a receptor ligand such as a hormone, growth factor, small molecule, cytokine, etc., and observing a response. The response could be a transcriptional response which could be measured, e.g., using a reporter gene assay, or by measuring the level of a gene product (transcribed RNA or protein translated therefrom). A response could be, e.g., a proliferative response, a change in cell morphology or properties, etc.

The invention further provides compositions and methods for identifying compounds and/or genes that modulate (e.g., enhance, inhibit, or otherwise modify) function of a Cohesin-Mediator complex, e.g., compounds and/or genes modulate interaction between Cohesin and Mediator. The invention further relates to methods of using such compounds. In some embodiments, such compounds are useful in treating a disorder in which a function of the Mediator-Cohesin complex is perturbed (e.g., relative to a normally functioning complex). In some embodiments, such compounds are useful in treating a disorder in which the Mediator-Cohesin interaction is perturbed. In some embodiments, the inventive compositions and methods employ one or more Cohesin and Mediator components or fragments thereof. In some embodiments, one or more Cohesin and/or Mediator components are within a cell. In some embodiments, one or more Cohesin and/or Mediator components are isolated from a cell. In some embodiments, one or more Cohesin and/or Mediator components are recombinantly produced.

In some embodiments, a “Cohesin component” comprises or consists of a polypeptide whose amino acid sequence is identical to the amino acid sequence of a naturally occurring Cohesin core complex polypeptide, e.g., Smc1a, Smc3, Rad21, STAG1 (also called SA1), or STAG2 (also called SA2) polypeptide. In some embodiments the naturally occurring polypeptide is an Smc polypeptide. In some embodiments the naturally occurring polypeptide is a STAG polypeptide. In some embodiments, the naturally occurring Cohesin core complex polypeptide is not Rad21. In some embodiments, a Cohesin component comprises or consists of a polypeptide whose amino acid sequence is identical to the amino acid sequence of a naturally occurring Cohesin complex associated polypeptide, e.g., Nipb1. As used herein, a Cohesin complex associated polypeptide refers a polypeptide that interacts with a Cohesin core complex and facilitates its activity (e.g., contributes to loading/unloading of the complex) and does not in general include Mediator components, e.g., does not include Mediator components known in the art. In some embodiments, the naturally occurring polypeptide is not Rad21.

In some embodiments, a “Mediator component” comprises or consists of a polypeptide whose amino acid sequence is identical to the amino acid sequence of a naturally occurring Mediator complex polypeptide. The naturally occurring Mediator complex polypeptide can be, e.g., any of the approximately 30 polypeptides found in a Mediator complex that occurs in a cell or is purified from a cell (see, e.g., Conaway et al., 2005; Kornberg, 2005; Malik and Roeder, 2005). In some embodiments a naturally occurring Mediator component is any of Med1-Med 31 or any naturally occurring Mediator polypeptide known in the art. For example, a naturally occurring Mediator complex polypeptide can be Med6, Med7, Med10, Med12, Med14, Med15, Med17, Med21, Med24, Med27, Med28 or Med30. In some embodiments a Mediator polypeptide is a subunit found in a Med11, Med17, Med20, Med22, Med 8, Med 18, Med 19, Med 6, Med 30, Med 21, Med 4, Med 7, Med 31, Med 10, Med 1, Med 27, Med 26, Med14, Med15 complex. In some embodiments a Mediator polypeptide is a subunit found in a Med12/Med13/CDK8/cyclin complex.

In some embodiments a “naturally occurring polypeptide” is a polypeptide that naturally occurs in a eukaryote, e.g., a vertebrate, e.g., a mammal. In some embodiments the mammal is a human. In some embodiments the vertebrate is a non-human vertebrate, e.g., a non-human mammal, e.g., rodent, e.g., a mouse, rat, or rabbit. In some embodiments the vertebrate is a fish, e.g., a zebrafish. In some embodiments the eukaryote is a fungus, e.g., a yeast. In some embodiments the eukaryote is an invertebrate, e.g., an insect, e.g., a Drosophila, or a nematode, e.g., C. elegans. Any eukaryotic species is encompassed in various embodiments of the invention. Similarly a cell or subject can be of any eukaryotic species in various embodiments of the invention. In some embodiments, the sequence of the naturally occurring polypeptide is the sequence most commonly found in the members of a particular species of interest. One of skill in the art can readily obtain sequences of naturally occurring polypeptides, e.g., from publicly available databases such as those available at the National Center for Biotechnology Information (NCBI) website (e.g., GenBank, OMIM, Gene). See, e.g., Table S12, which provides chromosomal positions and exemplary NCBI RefSeq accession numbers for mRNA encoding human Mediator and Cohesin components and certain other polypeptides of interest herein. (It will be appreciated that due to the degeneracy of the genetic code, Mediator components and Cohesin components could be encoded by many different nucleic acid sequences.) It will be understood that in some instances a gene or polypeptide will have been assigned a different name in different species. One of skill in the art could select an appropriate homolog, e.g., an ortholog. It will also be understood that polypeptides according to the invention can exist in multiple isoforms, any of which are encompassed by and useful in the described invention.

In some embodiments, a “Cohesin component” is a variant Cohesin component. As used herein, a variant Cohesin component comprises or consists of a polypeptide whose amino acid sequence is at least 70%, 80%, 90%, 95%, 96%, 97%, 98%, 99%, 99.5%, or greater than 99.5% identical to the amino acid sequence of a naturally occurring Cohesin core complex polypeptide or Cohesin complex associated polypeptide over a length at least 70%, 80%, 90%, 95%, 99%, or 100% of the full length of the naturally Cohesin core complex occurring polypeptide or Cohesin complex associated polypeptide, wherein the sequence of the naturally occurring Cohesin core complex polypeptide or Cohesin complex associated polypeptide is the sequence most commonly found in the members of a particular species of interest. In some embodiments, a “Mediator component” is a variant Mediator component. As used herein, a variant Mediator component comprises or consists of a polypeptide whose amino acid sequence is at least 70%, 80%, 90%, 95%, 96%, 97%, 98%, 99%, 99.5%, or greater than 99.5% identical to the amino acid sequence of a naturally occurring Mediator complex polypeptide over a length at least 70%, 80%, 90%, 95%, 99%, or 100% of the full length of the naturally occurring Mediator complex polypeptide, wherein the sequence of the naturally occurring Mediator complex polypeptide is the sequence most commonly found in the members of a particular species of interest. The term “variant” applies to polypeptides of interest herein. For example, the sequence of a Smc1a, Smc3, Rad21, STAG1, STAG2, Nibp1, Med6, Med7, Med10, Med12, Med14, Med15, Med17, Med21, Med24, Med27, Med28 or Med30 polypeptide can consist of a naturally occurring sequence most commonly found in the members of a particular species of interest, or the polypeptide can be a variant Smc1a, Smc3, Rad21, STAG1, STAG2, Nibp1, Med6, Med7, Med10, Med12, Med14, Med15, Med17, Med21, Med24, Med27, Med28 or Med30 polypeptide.

In some embodiments, a sequence of a variant Cohesin or Mediator component comprises or consists of a sequence that differs from a naturally occurring sequence by no more than 1, 2, 3, 4, 5, 6, 7, 8, 9, 10, 15, 20, or 25 amino acids. For example, a sequence of a variant Cohesin or Mediator component could comprise or consist of a sequence generated by making no more than 1, 2, 3, 4, 5, 6, 7, 8, 9, 10, 15, 20, or 25 amino acid deletions, substitutions, or insertions in a naturally occurring sequence. In some embodiments, a variant sequence could comprise or consist of a sequence generated by making a number of amino acid deletions, substitutions, or insertions that is no more than 1%, 2%, 5%, or 10% of the number of amino acids in a naturally occurring sequence. In some embodiments, a variant retains at least some activity of a naturally occurring component found most commonly in a species of interest or has equivalent activity. One of skill in the art will be aware that such variants can often be generated by making conservative substitutions and/or by making substitution in poorly conserved regions of a polypeptide.

“Identity” refers to the extent to which the sequence of two or more nucleic acids or polypeptides is the same. In some embodiments, percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used, A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

In some embodiments, the sequence of a variant Cohesin or Mediator component comprises or consists of a naturally occurring variant sequence, i.e., a naturally occurring sequence that differs from the sequence most commonly found in a species of interest. In some embodiments, the naturally occurring variant sequence is present in less than 1% of the members of a species of interest and may be referred to as a “mutant sequence”. In some embodiments, the naturally occurring variant sequence is not known to be associated with a disorder. In some embodiments, the naturally occurring variant sequence is known to be associated with a disorder. In some embodiments, a mutant sequence is inherited while in other embodiments a mutant sequence is found in an individual but is not present in the genome of the individual's parents. In some embodiments, the sequence of a variant Cohesin or Mediator component comprises or consists of a sequence that does not occur in nature.

In some embodiments, the sequence of a variant Cohesin or Mediator component comprises a sequence 100% identical to the sequence of the corresponding naturally occurring polypeptide found most commonly found in the members of a particular species of interest and further comprises one or more additional amino acids. For example, the variant could be a fusion protein that comprises a polypeptide sequence found in a different polypeptide, or a synthetic polypeptide sequence. In some embodiments, a variant comprises a “tag”, which term refers to a moiety appended to another entity that imparts a characteristic or property otherwise not present in the un-tagged entity. In some embodiments, the tag is an affinity tag, an epitope tag, a fluorescent tag, etc. Examples of fluorescent tags include GFP and other fluorescent proteins. Affinity tags can facilitate the purification or solubilization of fusion proteins. Examples of affinity tags include maltose binding protein (MBP), glutathione-S-transferase (GST), thioredoxin, polyhistidine (also known as 6×His), etc. Examples of epitope tags, which facilitate recognition by antibodies, include c-myc tag, FLAG (FLAG octapeptides), HA (hemagglutinin), etc. Biotin/streptavidin can also be used.

In some aspects, the invention relates to fragments of a Cohesin component, e.g., a portion or domain of a Cohesin component that mediates physical interaction with Mediator. In some aspects, the invention relates to fragments of a Cohesin component, e.g., a portion or domain of a Mediator component that mediates physical interaction with Cohesin. Such fragments are of use in various methods of the invention.

The invention provides a method of identifying a compound that modulates an interaction between Cohesin and Mediator comprising: (a) contacting a composition comprising at least one Cohesin component and at least one Mediator component with a test compound; (b) assessing the level of interaction between Cohesin and Mediator that occurs in the composition; and (c) comparing the level of interaction measured in step (b) with a suitable reference value, wherein if the level of interaction measured in step (b) differs from the reference value, the test compound modulates the interaction between Cohesin and Mediator. In some embodiments, “interaction” refers to a physical interaction, e.g., binding. In some embodiments such interaction is sufficiently strong and stable such that a complex comprising the Cohesin component and the Mediator component can be isolated, e.g., under appropriate conditions. In some embodiments a suitable reference value refers to a value that would exist in the absence of the test compound (or in the presence of a compound in an amount that has been previously shown not to affect the level of interaction). An increase in the level of interaction indicates that the compound enhances the interaction between Cohesin and Mediator. A decrease in the level of interaction indicates that the compound inhibits the interaction between Cohesin and Mediator. In some embodiments, a suitable reference value refers to a value that would exist in the presence of a compound that has been previously shown to affect the level of interaction.

In some embodiments, the Cohesin component(s) comprise a Smc1 or Smc3 polypeptide. In some embodiments, the Cohesin component(s) comprise a Nibp1 polypeptide. In some embodiments the Cohesin components comprise a Smc1, Smc3, and Nibp1 polypeptide. In some embodiments, the Mediator component(s) comprise a Med1 or a Med12 polypeptide. In some embodiments, the Mediator components comprise Med6, Med7, Med10, Med12, Med14, Med15, Med17, Med21, Med24, Med27, Med28 and Med30 polypeptides. In some embodiments, the composition comprises at least Med11, Med17, Med20, Med22, Med 8, Med 18, Med 19, Med 6, Med 30, Med 21, Med 4, Med 7, Med 31, Med 10, Med 1, Med 27, Med 26, Med14, Med15 polypeptides and, optionally, the components found in the Med12/Med13/CDK8/cyclin complex. In some embodiments, the composition comprises a purified Mediator complex. In some embodiments the composition comprises a cell (or, typically, multiple cells), tissue, organ, cell or tissue lysate or fraction thereof, e.g., a nuclear fraction or nuclear extract. In some embodiments the cell or tissue lysate or fraction thereof comprises all Cohesin and Mediator components that occur naturally in a cell of that species and cell type. In some embodiments, the Cohesin and Mediator component(s), e.g., complexes are at least partially purified from a cell or tissue lysate or fraction thereof. Any of a wide variety of cells can be used as sources for a Mediator and Cohesin component or for other purposes of the present invention. In some embodiments the cells are pluripotent cells, e.g., embryonic stem (ES) cells or induced pluripotent stem (iPS) cells. In some embodiments the cells comprise primary cells. The primary cells may have been maintained in culture prior to use. In some embodiments the cells comprise cells of a cell line, which may be an immortalized cell line. In some embodiments the cells are somatic cells. The cells could comprise cells of any cell type in various embodiments of the invention. In some embodiments the cells are isolated from a subject who has a disorder of interest or are descended from such cells. In some embodiments the cells comprise tumor cells. In some embodiments the cells comprise genetically engineered cells. In some embodiments, at least one of the components is a recombinant polypeptide, which may be produced by a genetically engineered cell or organism. In some embodiments, the cell(s) are contacted with the compound while in culture. The compound may be added to the culture medium. In other embodiments, the cells are contacted with the compound in vivo, e.g., the cells are cells of a multi-cellular organism, e.g., a human or non-human vertebrate subject, and contacting the cells comprises administering the compound to the organism. A biological sample comprising cells is obtained from the organism. Cells from the sample are used in the inventive method.

A variety of methods known in the art can be used to assess (e.g., detect and, optionally quantify) the level of interaction between Mediator and Cohesin or between components thereof. In some embodiments a Cohesin or Mediator component is isolated by a suitable method. Methods for isolating proteins and protein complexes are known in the art. It will be appreciated that the isolation should be performed using conditions suitable to maintain a protein complex. In some embodiments a method comprises contacting the composition with an agent (binding agent) that specifically binds to the Cohesin component or the Mediator component, respectively. In some embodiments a binding reagent, e.g., antibody, binds to a polypeptide that associates with Mediator, e.g., a co-activator, e.g., SREBP-1a, Material that binds to the binding agent (and material that is physically associated with material that is directly bound to the agent) is isolated, and the presence of one or more Cohesin and/or Mediator components in the isolated material is assessed. For example, if an agent that binds to a Mediator component is used, the presence of a Cohesin component in the isolated material may be assessed. If an agent that binds to a Cohesin component is used, the presence of a Mediator component in the isolated material may be assessed.

Methods for detecting and, optionally, quantifying proteins are known in the art and can be used in methods of the invention. For example, affinity-based methods, e.g., immunologically based methods such as ELISA, Western blot, or protein arrays, and the like can be used. Chromatography and/or mass spectrometry can be used. In some embodiments, a Cohesin or Mediator component comprises a detectable moiety, which may facilitate detection of the component. A detectable moiety can be, e.g., a fluorescent molecule, e.g., a polypeptide such as green fluorescent protein (GFP) or derivatives thereof, luminescent materials, bioluminescent materials, a tag, an enzyme, a radiolabel, etc. In some embodiments, interaction between a Cohesin component and a Mediator component is detected and, optionally, quantified, using FRET or BRET or similar techniques.

In some embodiments, a two hybrid screen is used to assess interaction between a Mediator component and a Cohesin component and/or to identify compounds that modulate the interaction.

In some embodiments, the function of a Cohesin-Mediator complex and/or the level of interaction is measured by assessing expression of a gene whose expression depends at least in part on a Cohesin-Mediator complex. Methods for assessing gene expression are well known in the art and include, e.g., Northern blots, microarrays, RT-PCR, and high throughput sequencing (e.g., RNA-Seq technology).

In some embodiments, the level of interaction is measured by detecting a DNA loop formed by Mediator and Cohesin, e.g., using 3C technology or the like.

In some embodiments, the level of interaction is measured by detecting co-occupancy of a promoter or enhancer by Mediator and Cohesin. Such co-occupancy can be assessed, e.g., using chromatin immunoprecipitation (ChIP) followed by microarray hybridization (ChIP-on-Chip) or followed by sequencing (ChIP-Seq). Some suitable methods are described herein.

In other embodiments, the effect of the compound on function or the level of interaction is assessed by assessing the effect of the compound on the pluripotency state of a pluripotent cell. As described herein, a number of Cohesin and Mediator components were identified in a screen for genes that contribute to maintenance of embryonic stern cell state. Short hairpin RNAs targeting these components were found to produce loss of ES cell state as evidenced by (i) reduced levels of Oct4 protein, (ii) a loss of ES cell colony morphology, (iii) reduced levels of mRNAs specifying transcription factors associated with ES cell pluripotency (e.g., Oct4, Sox2 and Nanog) and (iv) increased expression of mRNAs encoding developmentally important transcription factors (e.g., at least 3, 5, 10, 20, 30, or more TFs can be assessed). Such phenotypes are referred to herein as “loss of pluripotency” (LOP) phenotypes. It will be understood that the foregoing list is non-limiting. Other phenotypes associated with pluripotency or loss thereof could be used. For example, microRNAs are of interest. miRNA genes have been connected to the core transcriptional circuitry of ES cells (Marson A, Connecting microRNA genes to the core transcriptional regulatory circuitry of embryonic stem cells. Cell. 134(3):52′-33, 2008.), and have been identified as playing important roles in development. Thus alterations in miRNA expression profile could be used in certain embodiments to detect a loss or alteration in cell state.

Accordingly, compounds that modulate the function of a Cohesin-Mediator complex and/or that modulate the level of interaction between a Cohesin and a Mediator component can be identified by assessing the effect of such compounds on one or more phenotypes indicative of pluripotency or its loss (e.g., as described further below). A compound that inhibits certain functions of a Cohesin-Mediator complex, e.g., inhibits interaction between Cohesin and Mediator would at least in part mimic the result of shRNA knockdown of one or more Cohesin and/or Mediator components. For example, a compound that enhances the interaction may at least in part counteract the effect of a partial knockdown in a pluripotent cell into which such shRNAs have been introduced. It will be appreciated that an shRNA that produces only a partial knockdown (i.e., a reduction of expression of less than 100%) can be used if desired. One of skill in the art could select an shRNA producing a suitable level of knockdown such that an enhanced interaction could be detected. In some embodiments an inducible shRNA is used. Thus in some embodiments, the Cohesin component and the Mediator component are contacted with the test compound within a pluripotent cell, and the level of interaction is measured by detecting a loss of pluripotency (LOP) phenotype of the cell, wherein the LOP phenotype indicates that the compound disrupts interaction between Cohesin and Mediator.

In some aspects the invention provides methods of identifying a compound that affects cell state. In some aspects, a method comprises identifying a compound that modulates function of a Cohesin-Mediator complex. In some embodiments, the method comprises identifying a compound the interaction between Cohesin and Mediator. Methods for identifying such a compound are described herein. As described herein, Cohesin and Mediator are important regulators of cell state and form cell-type specific complexes with cell-type specific transcription factors. Through their roles in DNA loop formation at a subset of active promoters, Mediator and Cohesin link gene expression with cell-type specific chromatin structure. Accordingly, compounds that modulate (e.g., enhance, inhibit, modify) the Cohesin-Mediator complex can affect cell state. For example, in certain embodiments, compounds that modulate (e.g., enhance, inhibit, modify) the interaction between Mediator and Cohesin can affect cell state. In some embodiments, the cell state is characteristic of a cell type of interest. Optionally, the method comprises identifying a compound that modulates function of a Cohesin-Mediator complex in a cell of that cell type. The compound may or may not modulate the function in cells of a different type. Optionally, the method comprises identifying a compound that modulates the interaction between Cohesin and Mediator in a cell of that cell type. The compound may or may not modulate the interaction in cells of a different type. In some embodiments the cell state is characteristic of a disorder. For example, the disorder could be a proliferative disorder, wherein the state could be a state of cell proliferation or a state of cell cycle arrest. The disorder could be a developmental disorder. The cell state could be evidenced, e.g., by a distinctive gene expression profile. In the case of a disorder, the state can differ from a “normal” state. Some suitable methods for identifying such compounds are described herein. Other disorders of interest include, e.g., cardiovascular, psychiatric, neurodegenerative, musculoskeletal, autoimmune, infectious, metabolic, and other disorders. In some embodiments, a cell is in a state in which the cell contributes to the disorder, such as a proliferating state of a tumor cell, a pro-inflammatory state of a lymphocyte (e.g., a T cell) in a subject suffering from an inflammatory condition. In some embodiments, modulating the function of a Cohesin-Mediator interaction shifts the cell out of a state in which it contributes to the disorder.

In some embodiments of the invention, a cell in which a Cohesin-Mediator function is altered (e.g., reduced or increased), e.g., as compared with a normal cell, is used for compound screening. For example, in some embodiments a cell with a mutation in a Cohesin component or Mediator component is used, while in some embodiments a cell in which a Cohesin component (e.g., Nipb1) or Mediator component is inhibited (e.g., using RNAi or a small molecule) or increased (e.g., by expressing the component intracellularly) is used. In some embodiments, the altered Cohesin-Mediator function alters (a) binding of a Cohesin complex to Mediator complex or binding of a Cohesin component to a Mediator component; (b) occupancy of a cell type specific gene by Cohesin-Mediator complex; (c) expression or activity of a cell type specific gene; and/or (d) response of the cell to a signal transduction pathway. In some embodiments, the screening is to identify a compound that promotes or inhibits modification of the cell's state or type. In some embodiments, the screening is to identify a compound that at least in part counteracts or compensates for altered Cohesin-Mediator function. For example, in some embodiments the screening is to identify a compound that at least in part restores (a) binding of a Cohesin complex to Mediator complex or binding of a Cohesin component to a Mediator component; (b) occupancy of a cell type specific gene by Cohesin-Mediator complex; (c) expression or activity of a cell type specific gene; and/or (d) response to a signal transduction pathway. Such compounds may be used, e.g., to treat subjects suffering from disorders in which Cohesin-Mediator function is altered. The invention encompasses (a) contacting cells with (i) a first compound that alters (e.g., inhibits or increases) Cohesin-Mediator function and (ii) a test compound; and (b) determining whether the test compound at least in part counteracts or compensates for the effect of the first compound. If the test compound at least in part counteracts or compensates for the effect of the first compound, the compound is a candidate for treating a disorder associated with altered Cohesin-Mediator function. In some embodiments, the screening is to identify a compound that acts additively or synergistically with an inhibitor or enhancer of Cohesin-Mediator function to promote or inhibit modification of a cell's state or type.

In addition to identifying Mediator and Cohesin components as modifiers of ES cell state, a number of additional genes whose inhibition results in loss of ES cell state were identified. These genes (including the genes encoding Cohesin and Mediator components as described herein) are referred to herein as maintenance of pluripotency (“MOP”) genes. In one aspect, the invention provides a method of identifying a compound that affects cell state comprising: (a) providing a pluripotent cell that expresses a maintenance of pluripotency (MOP) gene, wherein the MOP gene is a gene whose inhibition results in at least one phenotype indicative of loss of pluripotency (LOP phenotype); (b) contacting the cell with a test compound; (c) inhibiting the MOP gene; (d) determining whether the cell exhibits at least one LOP phenotype, wherein if the cell fails to exhibit at least one LOP phenotype as compared to a suitable control, the compound affects cell state. One or more LOP phenotypes can be evaluated, and the list is non-limiting. It will be appreciated that failure to exhibit a phenotype indicative of loss of pluripotency is equivalent to maintaining/retaining a phenotype indicative of pluripotency. It will also be understood that the extent of such loss or maintenance can vary. One of skill in the art will set a suitable threshold for determining that a cell exhibits a phenotype indicative of loss of pluripotency and/or retains a phenotype indicative of pluripotency. For example, if the phenotype is loss or retention of 004 expression, one of skill in the art can determine whether a deviation from a control value is significant. In some embodiments, the LOP phenotype of step (a) and step (d) are the same. In some embodiments, the LOP phenotype of step (a), step (d), or both, is expression of Oct 4 protein. In some embodiments, the at least one transcription factor associated with pluripotency is selected from the group consisting of Oct 4, Nanog, and Sox2. In some embodiments, expression of the MOP gene is inhibited using RNA silencing, e.g., RNA interference (RNAi). RNAi can be accomplished using a suitable RNAi agent, e.g., a short interfering RNA (siRNA) or short hairpin RNA (shRNA). For example, in some embodiments, the cell comprises a nucleic acid that encodes a shRNA targeted to the MOP gene, wherein expression of the shRNA is regulatable, e.g., inducible, and inhibiting the MOP gene comprises inducing expression of the shRNA. “Inducible” is used in a general sense to indicate causing the siRNA to be expressed and does not imply a particular mechanism. For example, relieving repression of a gene that has been repressed by a small molecule (such as by switching a cell to medium lacking the repressor) could be considered “induction”. In some embodiments, the MOP gene is listed in Table S2. In some embodiments, the MOP gene encodes a transcriptional cofactor. In some embodiments the MOP gene encodes a chromatin regulator (e.g., a histone acetyltransferase or histone deacetylase or a histone methyltransferase or histone demethylase). In some embodiments, the embodiments, the MOP gene encodes a Cohesin or Mediator component.

Table S10 shows that modulating Cohesin-Mediator function has an effect on expression of certain developmental regulators. The list shows genes that fall into the Gene Ontology category Cellular Developmental Processes and in which the Smc1a and/or Med12 knockdowns caused their expression to increase at least 2-fold in ES cells. In some aspects, modulating a Cohesin-Mediator function modulates expression of one or more of the genes listed in Table S10.

In some embodiments, a pluripotent cell used in an inventive method herein is an embryonic stem (ES) cell. In some embodiments, a pluripotent cell is an induced pluripotent stem (iPS) cell. One of skill in the art will be aware that an iPS cell is a pluripotent somatic cell that has been derived from a non-pluripotent somatic cell (or is descended from a cell that has been so derived). An iPS cell can be derived using a variety of different protocols, many of which involve causing the cell to express at least the pluripotency factors Oct4, Nanog, and Sox2. Optionally the cells are caused to overexpress c-Myc. Examples of reprogramming factors of interest for reprogramming somatic cells to pluripotency in vitro are Oct4, Nanog, Sox2, and Lin28 are another combination of transcription factors useful to reprogram cells to pluripotency. A variety of techniques, e.g., involving small molecules and/or protein transduction have been employed in the generation of iPS cells, e.g., to replace at least one of the factors. See, e.g., PCT/US2008/004516 (WO 2008/124133) REPROGRAMMING OF SOMATIC CELLS); Lyssiotis, Calif., Proc Natl Acad Sci USA. 2009 Jun. 2; 106(248912-7. Epub 2009 May 15; Carey B W, Proc Natl Acad Sci USA. 2009 Jan. 6; 106(1):157-62. Epub 2008 Dec. 24, and references cited in any of the foregoing, for additional information regarding iPS cells. The invention contemplates use of any of the compositions and methods described in PCT/US2009/057692, “Compositions and Methods for Enhancing Cell Reprogramming”, filed 21 Sep. 2009.

In some aspects, the invention provides a method of identifying a compound that modifies chromatin architecture comprising the step of: identifying a compound that modulates the interaction between Cohesin and Mediator. Some suitable methods for identifying such compounds are described herein. In some embodiments, the compound modifies chromatin architecture in a cell-type specific manner, i.e., the compound has different effects on chromatin architecture in different cell types. Cell types, as used herein, could be (but are not limited to) any of the approximately 200 commonly recognized (e.g., in standard histology textbooks) and/or most abundant fully differentiated cell types found in an adult human (or comparable cells found in non-human animals). Examples include, e.g., neurons, lymphocytes, keratinocytes, hepatocytes, etc. In some embodiments, a cell type could also be a precursor or progenitor cell, e.g., a neural or hematopoietic progenitor cell. In some embodiments a cell is a fibroblast.

In some aspects, the invention provides methods of identifying a candidate compound for treating a disorder. As used herein, the term “disorder” refers to a disease, condition, syndrome, etc., recognized in the art. In some embodiments the disorder affects humans. In some embodiments, the disorder is a developmental disorder, e.g., the disorder manifests before the age of 18 and affects physical and/or mental development of children having the disorder, often resulting in multiple structural and/or functional abnormalities. Often a developmental disorder manifests within the first 2 years of life. In some embodiments, the disorder comprises an impairment in the growth and development of the brain or central nervous system. As used herein the term “developmental disorder” often excludes conditions caused by infectious agents, injuries, nutritional deficiencies, toxic agents, and tumors. In some embodiments, the disorder, e.g., developmental disorder, is a hereditary disorder, e.g., propensity to develop the disorder can be inherited. In some embodiments, the disorder can be inherited in a Mendelian manner. In some embodiments, the disorder is included among the disorders mentioned in the Online Mendelian Inheritance in Man® (OMIM) database, e.g., as of Feb. 8, 2010. OMIM is a compendium of human genes and genetic phenotype that contain information on all or the great majority of known Mendelian disorders and over 12,000 genes. In some embodiments, the disorder is a hereditary disorder, e.g., propensity to develop the disorder can be inherited.

Certain aspects of the invention relate to disorders, e.g., human disorders, that are associated with mutations in one or more Cohesin or Mediator components. As used herein, a “Cohesin-associated disorder” is a disorder associated with mutations in one or more Cohesin components. As used herein, a “Mediator-associated disorder” is a disorder associated with mutations in one or more Mediator components. In some embodiments, the disorder is one in which mutations in such component(s) have been highly correlated with developing the disorder. In some embodiments the mutation is one that is accepted in the art as likely to play a causative role in the disorder in at least some subjects. Not all subjects with a Cohesin-associated disorder may have a mutation in a Cohesin component. For example, in some embodiments the disorder is one in which it is estimated that at least about 10% of individuals having the disorder have a mutation in a Cohesin component. Different subjects may have mutations in different Cohesin components. Not all subjects with a Mediator-associated disorder may have a mutation in a Mediator component. For example, in some embodiments the disorder is one in which it is estimated at least about 10% of individuals having the disorder have a mutation in a Mediator component. Different subjects may have mutations in different Mediator components. A mutation could be in a transcribed region of a gene (e.g., a coding region) or an untranscribed region of the gene. In some embodiments a mutation is in a regulatory region of a gene, e.g., an enhancer or promoter.

Based on the instant invention, a disorder identified initially as being a Cohesin-associated disorder can also be a Mediator-associated disorder, and/or a disorder identified initially as being a Mediator-associated disorder can also be a Cohesin-associated disorder. For purposes of the instant invention, a disorder can be classified as “Cohesin-associated” or “Mediator-associated” based on whether it was first identified as being associated with mutations in Cohesin component(s) or Mediator component(s) respectively.

In some embodiments, the invention relates to Cornelia de Lange Syndrome (CdLS). Cornelia de Lange Syndrome is a developmental disorder characterized by a distinctive facial appearance, growth deficiency, and malformation of the upper extremities affecting 1 in 10,000 to 30,000 newborns. Mutations in Cohesin-related proteins have been identified in 65% of patients with CdLS with the following distribution: NIPBL, (60%), SMC1 (5%) and SMC3 (one case). CdLS is thus an exemplary Cohesin-associated disorder. Despite a well-established function of Cohesin in sister chromatid cohesion during cell cycle, CdLS patients do not show any mitotic defect. ChIP-Seq experiments performed by the instant inventors suggested the existence of at least two distinct cohesin-containing complexes: 1) the expected complex centered on CTCF containing Smc1, Smc3, Stag and Rad21 and 2) a complex containing Smc1, Smc3, Nipb1, Mediator and cell-type-specific transcription factors. The invention encompasses the recognition that these two complexes are respectively maintaining the sister chromatid cohesion and regulating transcription. Surprisingly, Nipb1 was found exclusively in the cohesin-containing transcription-specific complex at active genes. Co-immunoprecipitation revealed a strong association of Nipb1 with the general transcription factor TBP and other cell-type-specific regulators. The presence of Nipb1 in this complex explains the prevalence of human NIPBL mutation as well as the absence of mitotic defect observed in patient with CdLS. Destabilization of this transcription-specific Cohesin complex (e.g., physical destabilization of the complex and/or functional destabilization such that function of the complex is perturbed) is most likely to be the molecular explanation for gene dysregulation in CdLS and modulation of its function represents a novel pathway for drug development. Furthermore, Mediator mutations have been associated with Opitz-Kaveggia (FG) syndrome, Lujan syndrome, schizophrenia and some forms of congenital heart failure. These disorders are exemplary Mediator-associated disorders. The invention encompasses the recognition that these diseases, i.e., CdLS, Opitz-Kaveggia (FG) syndrome, Lujan syndrome, certain forms of schizophrenia and congenital heart failure, among others, are likely caused by defects in the Cohesin-Mediator interaction and/or defects in the Cohesin-Mediator complex described herein (e.g., defects resulting in altered function of the complex).

The invention further encompasses the recognition that genes that affect ES cell state are a source of candidate genes for human developmental disordes, i.e., genes that may harbor alterations, e.g., mutations, in subject(s) suffering from a human developmental disorder. Such genes include genes whose inhibition results in loss of a pluripotent state (or, in some embodiments, genes whose inhibition increases the manifestation of a phenotype associated with pluripotency or renders a cell resistant to an event that would otherwise be expected to lead to loss of pluripotency). Accordingly, compounds that modulate ES cell state, e.g., compounds that modulate Cohesin-Mediator function, e.g., by modulating a Cohesin-Mediator interaction, and/or render a cell able to retain pluripotency in spite of inhibition of a Cohesin or Mediator component, are candidate compounds for treating such disorders.

As used herein, “treat” or “treating” can include amelioration (e.g., reducing one or more symptoms of a disorder), cure, and/or maintenance of a cure (i.e., the prevention or delay of recurrence) of a disorder, or preventing a disorder from manifesting as severely as would be expected in the absence of treatment. Treatment after a disorder has started aims to reduce, ameliorate or altogether eliminate the disorder, and/or at least some of its associated symptoms, to prevent it from becoming more severe, to slow the rate of progression, or to prevent the disorder from recurring once it has been initially eliminated. Treatment can be prophylactic, e.g., administered to a subject that has not been diagnosed with the disorder, e.g., a subject with a significant risk of developing the disorder. For example, the subject may have a mutation associated with developing the disorder. In some embodiments, e.g., in the case of a disorder diagnosed prior to birth, treatment can comprise administering a compound to a subject's mother. In some embodiments, a method of the invention comprises providing a subject in need of treatment for a disease of interest herein, e.g., a developmental disorder or a proliferative disease. In some embodiments, a method of the invention comprises selecting a subject in need of treatment for a disease of interest herein, e.g., a developmental disorder or a proliferative disease. In some embodiments, a method of the invention comprises diagnosing a subject as having or being at risk of developing a disorder and, optionally, treating the subject. Certain inventive methods relating to diagnosis are described below. In some embodiments, a subject diagnosed or treated according to the instant invention is a human. In some embodiments a compound identified according to the invention is administered for veterinary purposes, e.g., to treat a vertebrate, e.g., domestic animal such as a dog, cat, horse, cow, sheep, etc.

Certain suitable methods for identifying a compound that modulates a function of a Cohesin-Mediator complex, e.g., a compound that modulates a Cohesin-Mediator interaction are described herein. In some aspects, a compound that modulates a Cohesin-Mediator function, e.g., a compound that modulates a Cohesin-Mediator interaction is a candidate compound for treating a disorder associated with a mutation in Cohesin or Mediator. For example, if the mutation results in diminished activity of a Cohesin-Mediator complex (e.g., as in the case of many mutations found in individuals with Cohesin-associated disorders), a compound that enhances, promotes, or maintains the interaction may be of benefit. If the mutation results in an aberrant gain of function of a Cohesin-Mediator complex, a compound that inhibits (reduces, decreases) the interaction may be of benefit. In one aspect, a compound that enhances a Cohesin-Mediator interaction and/or increases stability of a Cohesin-Mediator complex is a candidate compound for treating a disorder associated with mutations in a Cohesin or Mediator component.

In some aspects, a method of the invention comprises administering a compound identified as described herein to an animal model of a disorder. Animal models for a number of developmental disorders are known. For example, an animal model could be a mouse with a knockdown, knockout, or mutation in a Cohesin or Mediator component. In some embodiments, such knockout, knockdown, or mutation is heterozygous. In some embodiments, the animal is transgenic for an shRNA that inhibits expression of a Cohesin or Mediator component, optionally in a regulatable manner. In one aspect, an animal model, has a knockout, knockdown, or mutation in a Nibp1 gene, wherein the knockout, knockdown, or mutation reduces functional Nibp1 activity in at least some, e.g., most or all cells of the animal. See, e.g., Kawauchi S, et al., PLoS Genet. Multiple organ system defects and transcriptional dysregulation in the Nipb1(+/−) mouse, a model of Cornelia de Lange Syndrome. 2009 September; 5(9):e1000650. Epub 2009 Sep. 18. In one aspect, a compound identified according to the invention is tested using such an animal model. For example, the effect of the compound on one or more phenotypic features and/or gene expression can be assessed. A compound that lessens, ameliorates, and/or at least partially normalizes any of the distinctive features of such animal model is a promising candidate to treat the disorder.

The invention encompasses the recognition that the state of a cell, e.g., with respect to proliferation, may be influenced by the Cohesin-Mediator complex described herein. The invention further encompasses the recognition that ES cells and cancer stem cells share many characteristics including a high proliferation rate and a low differentiation level. The invention encompasses the recognition that the dependency on a transcription-specific Cohesin-containing complex to maintain cell state should be conserved between normal cells and cancer cells, e.g., cancer stem cells. Certain aspects of the invention relate to targeting of this novel pathway for development of new therapies for cancer and other proliferative diseases. For example, in some embodiments, a compound that modulates a function of a Cohesin-Mediator complex is a candidate compound for treating a proliferative disease. In some embodiments, a compound that mimics the effect of a knockdown of a Cohesin or Mediator component (e.g., causes a LOP phenotype) is a candidate compound for treating a proliferative disease. In other embodiments, a compound that disrupts a Cohesin-Mediator interaction, e.g., in a tumor cell is a candidate compound for treating a proliferative disease. In some embodiments, a compound differentially affects, e.g., disrupts, a Cohesin-Mediator interaction in a tumor cell versus a normal cell. In some embodiments, a compound that modulates a Cohesin-Mediator function, e.g., in a tumor cell is a candidate compound for treating a proliferative disease. In some embodiments, a compound differentially affects a Cohesin-Mediator function in a tumor cell versus a normal cell. Proliferative diseases include a variety of disorders characterized by abnormal or unwanted cell proliferation or survival. In some embodiments, the proliferative disease is a solid tumor. In some embodiments, the proliferative disease is a hematological malignancy. In certain embodiments, the proliferative disease is a benign neoplasm. In other embodiments, the neoplasm is a malignant neoplasm. In certain embodiments, the proliferative disease is a cancer, which term as used herein includes carcinomas and sarcomas. Exemplary tumors include colon cancer, lung cancer (e.g., small cell lung cancer, non-small cell lung cancer), bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, breast cancer, gastric cancer, eye cancer, gallbladder cancer, laryngeal cancer, oral cancer, penile cancer, glandular tumors, rectal cancer, small intestine cancer, gastrointestinal stromal tumors (GISTs), sarcoma, carcinoma, melanoma, urethral cancer, vaginal cancer, to name but a few. In some embodiments, a cancer is a hematological malignancy. In some embodiments, the hematological malignancy is a lymphoma. In some embodiments, the hematological malignancy is a leukemia. Examples of hematological malignancies include, but are not limited to, acute lymphoblastic leukemia (ALL), acute myelogenous leukemia (AML), chronic myelogenous leukemia (CML), chronic lymphocytic leukemia (CLL), hairy cell leukemia, Hodgkin's lymphoma, non-Hodgkin's lymphoma, cutaneous T-cell lymphoma (CTCL), peripheral T-cell lymphoma (PTCL), Mantle cell lymphoma, B-cell lymphoma, acute lymphoblastic T cell leukemia (T-ALL), acute promyelocytic leukemia, and multiple myeloma.

In certain embodiments, the disorder, e.g., proliferative disease, is an inflammatory disease. In some embodiments the disorder is an autoimmune disease. In certain embodiments, the disorder is associated with pathologic neovascularization. Other proliferative diseases include, e.g., neurofibromatosis, atherosclerosis, pulmonary fibrosis, arthritis, psoriasis, hypertrophic scar formation, inflammatory bowel disease, post-transplantation lymphoproliferative disorder, etc. Other diseases of interest include infectious diseases, cardiovascular diseases, and neurodegenerative diseases.

In some aspects, a method of the invention comprises administering a compound identified as described herein to an animal model of a proliferative or other disorder. For example, the subject may have a tumor xenograft or may be injected with tumor cells or have a predisposition to develop tumors. In some embodiments the animal is immunocompromised. The non-human animal may be useful for assessing effect of an inventive compound on tumor formation, development, progression, metastasis, etc. In some embodiments the animal is used to assess efficacy and/or toxicity of a compound. Methods known in the art can be used for such assessment. In some embodiments, the subject may be a genetically engineered non-human mammal, e.g., a mouse, that has a predisposition to develop tumors. The mammal may overexpress an oncogene (e.g., as a transgene) or underexpress a tumor suppressor gene (e.g., the animal may have a mutation or deletion in the tumor suppressor gene).

In some aspects, the invention provides an isolated complex comprising a Cohesin component and a Mediator component. “Isolated” refers typically to a material or substance that is separated from at least some other materials or substances with which it is normally found in nature, usually by a process involving the hand of man, or is artificially produced, e.g., chemically synthesized, or present in an artificial environment (e.g., outside the body of a subject). In some embodiments, any of the nucleic acids, polypeptides, nucleic-acid-protein structures, protein complexes, cells, or compounds of the invention, is isolated. In some embodiments, an isolated nucleic acid is a nucleic acid that has been synthesized using recombinant nucleic acid techniques or in vitro transcription or chemical synthesis or PCR. In some embodiments, an isolated polypeptide is a polypeptide that has been synthesized using recombinant nucleic acid techniques or in vitro translation or chemical synthesis. In some embodiments an isolated complex is a complex that has been obtained from cells. In some embodiments, the complex is substantially free of CTCF, Rad21, or both. In some embodiments the isolated complex contains an Smc1 polypeptide, an Smc3 polypeptide, and/or a Nibp1 polypeptide, and multiple Mediator components. For example, the complex can contain at least 10, 15, 20, 25, or more Mediator components. In some embodiments the complex contains, e.g., Med5, Med6, Med7, Med10, Med12, Med 14, Med15, Med17, Med21, Med24, Med27, Med28 and/or Med30, polypeptides, or a subset thereof. In some embodiments the complex comprises, e.g., in addition to the foregoing components, Med 6, Med8, and/or Med25. In some embodiments a complex comprises at least the core Mediator components as described in Malik & Roeder, 2005, and a CDK8/Cyclin C/Med12/Med13 subcomplex. In some embodiments a complex comprises those Mediator components that can be co-immunoprecipitated with one or more Cohesin components. In some embodiments, the Cohesin component is a variant Cohesin component and/or the Mediator component is a variant Mediator component. In some embodiments, the complex has been isolated using at least two binding agents, wherein a first binding agent binds to a Cohesin component and a second binding agent binds to a Mediator component or to a Mediator-associated protein. A “Mediator-associated protein” is a polypeptide such as SREBP-1a that is known in the art to bind to Mediator (for purposes herein, “Mediator-associated protein refers to polypeptides other than Cohesin components). In some embodiments, the Cohesin component, Mediator component, or both, is a recombinant protein. In some embodiments, a Cohesin component, Mediator component, or both, comprises a tag. For example, a Cohesin component could comprise a tag for purification, and a Mediator component could comprise a fluorescent tag for detection. In some embodiments, a Cohesin component and a Mediator component are cross-linked. In some embodiments, the complex (or at least one component thereof) is isolated from a cell derived from a subject who has a disorder of interest. As used herein, a cell “derived from a subject” refers to a cell obtained directly from the subject or a descendant thereof (i.e., a cell that is descended from the originally obtained cell). It will be understood that the phrase “obtained directly from a subject” encompasses situations in which the physical procedure of obtaining a biological sample comprising cells, e.g., a tissue sample or blood sample, from the subject is performed by the same individual or entity who uses the cell or a descendant thereof or subsequently practices an inventive method and situations in which a third party (e.g., a health care provider) takes a sample and then provides the sample (or cells from the sample) to another party such that the cell or a descendant thereof is eventually used in an inventive method. A cell may have been maintained in culture and/or maintained frozen for varying periods of time prior to use in an inventive method. For example, the cell may have been maintained for days, weeks, months, or longer, over many passages, e.g., between 1 and 50 passages, or more. In some embodiments, a cell is manipulated, e.g., genetically modified.

In some embodiments, the invention provides a composition comprising an isolated complex comprising a Cohesin component and a Mediator component, wherein the composition is substantially free of Cohesin components that are not complexed with Mediator components. In some embodiments the composition is substantially free of CTCF and/or Rad21. In some embodiments the isolated complex or composition containing it is substantially free of a Cohesin component required only for cohesion of sister chromatids during G2 and/or mitosis. In some embodiments, the complex or composition further comprises at least one general transcription factor, e.g., TBP, and/or one or more cell-type-specific regulators. In some embodiments, the composition is substantially free of Mediator components not complexed with Cohesin components. In some embodiments, the amount of one or more Cohesin component, one or more Mediator, or both, is quantified. In some embodiments, a complex or composition is “substantially free” of a polypeptide if the complex or composition comprises less than about 5%, or 2% of the polypeptide by dry weight or on a molar basis. In some embodiments, a complex or composition is “substantially free” of a polypeptide if the complex or composition comprises less than about 1%, 0.5%, or 0.1% of the polypeptide by dry weight or on a molar basis. In some embodiments, “substantially free” means that the polypeptide is not detectable using a Western blot. In some embodiments, a complex or composition is substantially free of a polypeptide if the molar ratio of Smc1 or Nipb1 to the polypeptide is at least 10:1, at least 20:1, or higher.

In some embodiments, the invention provides a composition comprising an isolated Cohesin component and an isolated Mediator component. In some embodiments, the Cohesin component, the Mediator component, or both, are in a complex (e.g., a Cohesin complex, Mediator complex, or Cohesin-Mediator complex, as described herein). The invention encomnpasses embodiments in which the composition comprises any one or more Cohesin components and any one or more Mediator components. In some embodiments, the composition further comprises any one or more of the following: (i) isolated DNA (e.g., promoter region DNA, enhancer region DNA, or both, optionally including at least part of a transcribed region of a gene); (ii) one or more transcription factor(s), e.g., cell-type specific transcription factor(s); (iii) one or more components of the transcription initiation apparatus (e.g., RNA polymerase II). In some embodiments, the Cohesin and Mediator components are physically associated with one or more transcription factor(s). In some embodiments, one or more transcription factor(s) is bound to DNA, e.g., DNA comprising an enhancer and/or transcription initiation apparatus is bound to DNA, e.g., DNA comprising a promoter. In some embodiments, the DNA is in the form of one or more segments of DNA about 5 kB, 2 kB, 1 kB, 500 bp, 250 bp, or less in size, e.g., between about 100 bp and about 2 kB.

In some embodiments, at least 50%, 60%, 70%, 75%, 80%, 85%, 90%, 95%, 96%, 97%, 98%, 99%, or more of the total polypeptide material in a composition of the invention comprises Mediator and Cohesin components. In some embodiments, at least 50%, 60%, 70%, 75%, 80%, 85%, 90%, 95%, 96%, 97%, 98%, 99%, or more of the total polypeptide material in a composition of the invention comprises Mediator components, Cohesin components, transcription factors, co-activators, and transcription apparatus. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules, and/or nucleic acid can optionally be present. In some embodiments, an isolated complex is at least in part assembled in vitro, e.g., by combining isolated components of the complex in the same vessel.

In some embodiments, the invention provides a method of characterizing a cell comprising: assessing function of a Cohesin-Mediator complex of the cell. The function can be, e.g., (a) binding of a Cohesin complex to Mediator complex or binding of a Cohesin component to a Mediator component; (b) occupancy of a cell type specific gene by Cohesin-Mediator complex; (c) expression or activity of a cell type specific gene; (d) response to a signal transduction pathway. In some embodiments, the result of the assessment provides information as to whether the Cohesin-Mediator complex is functioning normally. In some embodiments, the information is of use to diagnose a disorder, identify a compound, monitor the effect of a compound (e.g., monitor the effect of a therapy or determine whether a therapy is suitable for a subject), e.g., as described herein. In some embodiments the method comprises comparing the function with a reference value. It will be understood that certain methods of the invention, e.g., methods of characterizing a cell, analyzing a Cohesin-Mediator complex of a cell, or modulating function of a Cohesin-Mediator complex of a cell, may be practiced using a population of cells. A population of cells can be composed of largely or substantially identical cells, e.g., cells derived from a single ancestor cell or from a defined and/or substantially identical population of ancestor cells, e.g., so that the cells are substantially identical. In some embodiments a method may be practiced using a population of cells derived from an individual subject or descended from cells obtained from an individual subject (e.g., a sample obtained from a subject).

In some embodiments, the invention provides a method of characterizing a cell comprising: detecting an interaction between a Cohesin component and a Mediator component that takes place in the cell. The invention further provides a method of characterizing a cell comprising detecting an interaction between a Cohesin complex and a Mediator complex that takes place in a cell. In some embodiments, detection of an interaction occurs while the components and/or complexes are in the cell. In some embodiments, a complex is isolated from the cell, and the presence of one or more components in the complex is assessed. In some embodiments the complex is disrupted prior to detection.

The invention further provides a method of characterizing a cell comprising isolating a Cohesin-Mediator complex from a cell. In some embodiments the method further comprises detecting a Cohesin or Mediator component in the isolated complex. In some embodiments the complex is disrupted prior to detection.

The invention further provides a method of characterizing a cell comprising (a) isolating material comprising a Mediator component from a cell; and (b) detecting a Cohesin component in the isolated material. In some embodiments the method further comprises analyzing a Cohesin component and/or a Mediator component present in the isolated material. The material comprising a Mediator component can be isolated using any suitable method. It will be understood that the suitable method is not a method designed or specifically adapted for isolation of Cohesin or a Cohesin component. In some embodiments the material is isolated using an agent (e.g., an antibody) that binds to a Mediator component, Mediator complex, or that binds to a Mediator-associated protein. “Analyzing” could include assessing (e.g., detecting, quantifying) any one or more properties of a substance. In the case of a polypeptide, analyzing could encompass examining post-translational modification(s), binding ability, enzymatic activity, amount, etc.

The invention further provides a method of characterizing a cell comprising (a) isolating material comprising a Cohesin component from a cell; and (b) detecting a Mediator component in the isolated material. In some embodiments the method further comprises analyzing a Cohesin component and/or a Mediator component present in the isolated material. The material comprising a Cohesin component can be isolated using any suitable method. It will be understood that the suitable method is not a method designed or specifically adapted for isolation of Mediator or a Mediator component. In some embodiments the material is isolated using an agent (e.g., an antibody) that binds to a Cohesin component. “Analyzing” could include assessing (e.g., detecting, quantifying) any one or more properties of a substance. In the case of a polypeptide, analyzing could encompass examining post-translational modification(s), binding ability, enzymatic activity, amount, etc. In some embodiments the Cohesin component is Nibp1.

In some embodiments of any of the methods of characterizing a cell, a Mediator component or Cohesin component is a variant Mediator component or a variant Cohesin component, respectively. In some embodiments of any of the methods of characterizing a cell a Cohesin component or Mediator component is a recombinant protein and/or comprises a tag. In some embodiments of any of the methods of characterizing a cell, the cell is derived from a subject having or suspected of having a disorder of interest. Optionally, the method further comprises diagnosing the subject as having or not having the disorder based at least in part on analysis of a Cohesin or Mediator component or Cohesin-Mediator complex present in the isolated material, e.g., based at least in part on the amount or properties (e.g., functional and/or structural properties) of the component or complex. It will be understood that the diagnostic method may be used in conjunction with one or more clinical, laboratory-based or other diagnostic methods.

In another aspect, the invention provides a method of characterizing a cell derived from a subject having or suspected of having a Cohesin-associated disorder or a Mediator-associated disorder, comprising the step of determining whether the cell has an alteration in function of a Cohesin-Mediator complex as compared with a reference, e.g., a normal cell. In some embodiments, the method further comprises diagnosing the subject as having such a disorder based on the whether the cell has an alteration in function of a Cohesin-Mediator complex.

In another aspect, the invention provides a method of characterizing a cell derived from a subject having or suspected of having a Cohesin-associated disorder comprising the step of determining whether the cell has an alteration in a Mediator component or in a gene encoding a Mediator component, as compared with a reference. In some embodiments, the method comprises determining whether the cell has a mutation in a gene encoding a Mediator component. In some embodiments, the method comprises determining whether the cell has increased or decreased expression or post-translational modification of a Mediator component. In some embodiments, the method comprises determining whether the cell has altered binding of Mediator to at least one enhancer or promoter. In some embodiments, the method comprises determining whether the cell has altered interaction between Mediator and Cohesin. In some embodiments, the method comprises determining whether a Mediator or Cohesin component of the cell has an altered post-translational modification(s), binding ability, enzymatic activity, or amount.

The invention further provides a method of characterizing a cell derived from a subject having or suspected of having a Mediator-associated disorder comprising the step of determining whether the cell has an alteration in a Cohesin component or in a gene encoding a Cohesin component, as compared with a reference. In some embodiments, the method comprises determining whether the cell has a mutation in a gene encoding a Cohesin component. In some embodiments, the method comprises determining whether the cell has increased or decreased expression or post-translational modification of a Cohesin component. In some embodiments, the method comprises determining whether the cell has altered binding of Cohesin to at least one enhancer or promoter. In some embodiments, the method comprises determining whether the cell has altered interaction between Mediator and Cohesin. In some embodiments, the method comprises determining whether a Mediator or Cohesin component of the cell has an altered post-translational modification(s), binding ability, enzymatic activity (e.g., kinase activity) or amount. In some embodiments, the method comprises providing (e.g., obtaining) a sample from a subject. In some embodiments, the subject is suffering from or has at least one symptom or manifestation of a disorder, e.g., a Cohesin-associated disorder or a Mediator-associated disorder. The sample may be, e.g., a blood sample, skin biopsy, tissue sample, fine needle biopsy sample, surgical sample, or other type of sample containing cells. Optionally the method comprises culturing the cells, processing the cells to extract DNA, mRNA and/or protein(s), fixing or staining the cells, performing chromatin immunoprecipitation and/or chromosome conformation capture on the cells, analyzing binding of a Cohesin complex to a Mediator complex or binding of a Cohesin component to a Mediator component; analyzing transcription of one or more cell-type specific genes and/or analyzing occupancy of a cell type specific gene by Mediator and/or Cohesin.

Any suitable method can be used to determine whether a cell has a mutation in a gene encoding a Cohesin or Mediator component. For example, sequencing can be used to identify a mutation. A variety of methods can be used, e.g., after a mutation has been identified initially in one or more subjects having a disorder of interest. Such methods can, for example, employ a suitable probe or primer to selectively detect and/or amplify at least a portion of a mutant or non-mutant allele, allowing one to distinguish among different alleles. Detection can use an oligonucleotide array, e.g., a SNP array. Such arrays are available, e.g., from Affymetrix. Alternately, mutations that cause differences in a coding sequence can sometimes be detected using antibodies selective for a mutant or non-mutant form, or differences in molecular weight can be detected. Any methods known in the art for detecting mutations are within embodiments of the invention. Probes, primers, arrays, and other agents useful for detecting a mutation can be provided in a kit, which can contain instructions for use, reagents for performing an assay, etc.

The inventive methods can be used to diagnose or assist in diagnosis of a disorder. For example, without wishing to be bound by theory, a disorder that has been identified as a Cohesin-associated disorder may, in some subjects, be associated with a mutation in a Mediator component, wherein such mutation alters the activity of a Cohesin-Mediator complex identified herein. Likewise, a disorder that has been identified as a Mediator-associated disorder may, in some subjects, be associated with a mutation in a Cohesin component, wherein such mutation alters the activity of a Cohesin-Mediator complex identified herein.

In some embodiments of any of the methods for characterizing a cell derived from a subject having or suspected of having a disorder, the cell is of a type that shows evidence of the disorder and/or is of a type whose dysfunction contributes to the disorder. In some embodiments, the cell is of a type that does not show evidence of the disorder and/or is not of a type whose dysfunction is believed to contribute to the disorder.

In some embodiments of any of the inventive methods of characterizing a cell or sample, can comprise determining that a component or complex is present. In some embodiments, any of the inventive methods of characterizing a cell or sample can comprise determining that a component or complex is not present.

Certain embodiments of the present invention relate to and/or make use of a variety of different polypeptides. The terms “protein” and “polypcptide” are used interchangeably herein. In some embodiments, a polypeptide contains only the standard 20 amino acids found in proteins, although non-standard amino acids (e.g., compounds that do or do not occur in nature but that can be incorporated into a polypeptide chain) and/or amino acid analogs as are known in the art may alternatively be employed. One of skill in the art will appreciate that one or more amino acids of a polypeptide can be modified, e.g., by the addition of a chemical entity such as a carbohydrate group, a phosphate group, a farnesyl group, an isofarnesyl group, a fatty acid group. Such modification could occur post-translationally.

Various embodiments of the invention relate to and/or make use of genes and nucleic acids, e.g., genes and nucleic acids that encode a Cohesin component or Mediator component. As herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides. In some embodiments, a polypeptide of interest herein is encoded by a nucleic acid that encodes the polypeptide in nature. One of skill in the art can readily obtain such sequences (e.g, cDNA and/or mRNA sequences) and sequences encoding other polypeptides of interest herein from publicly available databases such as those available at the National Center for Biotechnology Information (NCBI) website (e.g., GenBank, OMIM). Furthermore, one of skill in the art can obtain genomic sequences containing the coding region and, optionally, regulatory elements, e.g., from genome databases (e.g., at the NCBI or the UCSC genome browser). It is expected that DNA sequence polymorphisms that may or may not lead to changes in the amino acid sequences of the polypeptide will exist among individuals in a species or population. One skilled in the art will appreciate that these variations in one or more nucleotides (e.g., up to about 3-5% of the nucleotides) of the nucleic acids encoding a particular polypeptide may exist among individuals of a given species or population due to natural allelic variation. All such nucleotide variations and resulting amino acid polymorphisms (if any) are within the scope of this invention and may be employed in various embodiments as appropriate.

Certain aspects of the invention relate to and/or make use of a genetically modified cell or organism. In some aspects, a cell or organism is genetically modified using a suitable vector. As used herein, a “vector” may comprise any of a variety of nucleic acid molecules into which a desired nucleic acid may be inserted, e.g., by restriction digestion followed by ligation. A vector can be used for transport of such nucleic acid between different environments, e.g., to introduce the nucleic acid into a cell of interest and, optionally, to direct expression in such cell. Vectors are often composed of DNA although RNA vectors are also known. Vectors include, but are not limited to, plasmids and virus genomes or portions thereof. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics or other compounds, enzymes whose activities are detectable by standard assays known in the art (e.g., β-galactosidase or alkaline phosphatase), and proteins or RNAs that detectably affect the phenotype of transformed or transfected cells (e.g., fluorescent proteins). An expression vector is one into which a desired nucleic acid may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Regulatory elements may be contained in the vector or may be part of the inserted nucleic acid or inserted prior to or following insertion of the nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of skill in the art will be aware that the precise nature of the regulatory sequences needed for gene expression may vary between species or cell types, but can in general include, as necessary, 5′ non-transcribed and/or 5′ untranslated sequences that may be involved with the initiation of transcription and translation respectively, such as a TATA box, cap sequence, CAAT sequence, and the like. Other regulatory elements include IRES sequences. Such 5′ non-transcribed regulatory sequences will include a promoter region that includes a promoter sequence for transcriptional control of the operably linked gene. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. One of skill in the art is aware of regulatable (e.g., inducible or repressible) expression systems such as the Tet system (e.g., the Tet-On or Tet-Off system) and others that can be regulated by small molecules and the like, as well as tissue-specific and cell type specific regulatory elements. In some embodiments, expression is regulatable using tetracycline, doxycline, or analogs thereof. In some embodiments expression is regulatable using a steroid hormone (e.g., estrogen) or analog thereof (e.g., tamoxifen). In some embodiments, a virus vector is selected from the group consisting of adenoviruses, adeno-associated viruses, poxviruses including vaccinia viruses and attenuated poxviruses, retroviruses (e.g., lentiviruses), Semliki Forest virus, Sindbis virus, etc. Optionally the virus is replication-defective. In some embodiments a replication-deficient retrovirus (i.e., a virus capable of directing synthesis of one or more desired transcripts, but incapable of manufacturing an infectious particle) is used. Various techniques may be employed for introducing nucleic acid molecules into cells. Such techniques include transfection of nucleic acid molecule-calcium phosphate precipitates, transfection of nucleic acid molecules associated with DEAE, transfection or infection with a virus that contains the nucleic acid molecule of interest, liposome-mediated transfection, nanoparticle-mediated transfection, and the like.

Certain embodiments of the invention relate to methods for identifying compounds that modulate (e.g., enhance, inhibit, or otherwise modify) the interaction between Cohesin and Mediator. The invention further relates to methods of using such compounds. Any of a wide variety of compounds can be used in the invention.

Compounds of use in various embodiments of the invention can comprise, e.g., small molecules, peptides, polypeptides, nucleic acids, oligonucleotides, etc. A small molecule is often an organic compound having a molecular weight equal to or less than 2.0 kD, e.g., equal to or less than 1.5 kD, e.g., equal to or less than 1 kD, e.g., equal to or less than 500 daltons and usually multiple carbon-carbon bonds. Small molecules often comprise one or more functional groups that mediate structural interactions with proteins, e.g., hydrogen bonding, and typically include at least an amine, carbonyl, hydroxyl or carboxyl group, and in some embodiments at least two of the functional chemical groups. A small molecule may comprise cyclic carbon or heterocyclic structures and/or aromatic or polyaromatic structures substituted with one or more chemical functional groups and/or heteroatoms. In some embodiments a small molecule satisfies at least 3, 4, or all criteria of Lipinski's “Rule of Five”.

Nucleic acids, e.g., oligonucleotides (which typically refers to short nucleic acids, e.g., 50 nucleotides in length or less), can be used. The invention contemplates use of oligonucleotides that are single-stranded, double-stranded (ds), blunt-ended, or double-stranded with overhangs, in various embodiments of the invention. The full spectrum of modifications (e.g., nucleoside and/or backbone modifications), non-standard nucleotides, delivery vehicles and systems, etc., known in the art as being useful in the context of siRNA or antisense-based molecules for research or therapeutic purposes is contemplated for use in various embodiments of the instant invention. In some embodiments a compound is an RNAi agent, antisense oligonucleotide, or aptamer. The term “RNAi agent” encompasses nucleic acids that can be used to achieve RNA silencing in eukaryotic, e.g., vertebrate, e.g., mammalian cells. As used herein RNA silencing, also termed RNA interference (RNAi), encompasses processes in which sequence-specific silencing of gene expression is effected by an RNA-induced silencing complex (RISC) that has a short RNA strand incorporated therein, which strand directs or “guides” sequence-specific degradation or translational repression of mRNA to which it has complementarity. The complementarity between the short RNA and mRNA need not be perfect (100%) but need only be sufficient to result in inhibition of gene expression. For example, the degree of complementarity and/or the characteristics of the structure formed by hybridization of the mRNA and the short RNA strand can be such that the strand can (i) guide cleavage of the mRNA in the RNA-induced silencing complex (RISC) and/or (ii) cause translational repression of the mRNA by RISC. RNAi may be achieved artificially in eukaryotic, e.g., mammalian, cells in a variety of ways. For example, RNAi may be achieved by introducing an appropriate short double-stranded nucleic acid into the cells or expressing in the cells a nucleic acid that is processed intracellularly to yield such short dsRNA. Exemplary RNAi agents are a short hairpin RNA (shRNA), a short interfering RNA (siRNA), micrRNA (miRNA) and a miRNA precursor. siRNAs typically comprise two separate nucleic acid strands that are hybridized to each other to form a duplex. They can be synthesized in vitro, e.g., using standard nucleic acid synthesis techniques. A nucleic acid may contain one or more non-standard nucleotides, modified nucleosides (e.g., having modified bases and/or sugars) or nucleotide analogs, and/or have a modified backbone, Any modification or analog recognized in the art as being useful for RNAi, aptamers, antisense molecules or other uses of oligonucleotides can be used. Some modifications result in increased stability, cell uptake, potency, etc. Exemplary compound can comprise morpholinos or locked nucleic acids. In some embodiments the nucleic acid differs from standard RNA or DNA by having partial or complete 2′-O-methylation or 2′-O-methoxyethyl modification of sugar, phosphorothioate backbone, and/or a cholesterol-moiety at the 3′-end. In certain embodiments the siRNA or shRNA comprises a duplex about 19 nucleotides in length, wherein one or both strands has a 3′ overhang of 1-5 nucleotides in length (e.g., 2 nucleotides), which may be composed of deoxyribonucleotides. shRNA comprise a single nucleic acid strand that contains two complementary portions separated by a predominantly non-self-complementary region. The complementary portions hybridize to form a duplex structure and the non-self-complementary region forms a loop connecting the 3′ end of one strand of the duplex and the 5′ end of the other strand. shRNAs can undergo intracellular processing to generate siRNAs. In certain embodiments the term “RNAi agent” also encompasses vectors, e.g., expression vectors, that comprise templates for transcription of an siRNA (e.g., as two separate strands that can hybridize), shRNA, or microRNA precursor, and can be used to introduce such template into cells and result in transient or stable expression thereof.

In some embodiments an RNAi agent, aptamer, antisense oligonucleotide, other nucleic acid, peptide, polypeptide, or small molecule is physically associated with a moiety that increases cell uptake, such as a cell-penetrating peptide, or a delivery agent. In some embodiments a delivery agent at least in part protects the compound from degradation, metabolism, or elimination from the body (e.g., increases the half-life). A variety of compositions and methods can be used to deliver agents to cells in vitro or in vivo. For example, compounds can be attached to a polyalkylene oxide, e.g., polyethylene glycol (PEG) or a derivative thereof, or incorporated into or attached to various types of molecules or particles such as liposomes, lipoplexes, or polymer-based particles, e.g., microparticles or nanoparticles composed at least in part of one or more biocompatible polymers or co-polymers comprising poly(lactide-glycolide), copolyoxalates, polycaprolactones, polyesteramides, polyorthoesters, polyhydroxybutyric acid, and/or polyanhydrides.

In some embodiments, a compound comprises a polypeptide or a nucleic acid encoding a polypeptide. A polypeptide can be a Cohesin or Mediator component. For example, a cell that expresses a variant Cohesin or Mediator component that has reduced or aberrant activity can be supplied with a nucleic acid encoding a normal version. In some embodiments a compound comprises an antibody. The term “antibody” encompasses immunoglobulins and derivatives thereof containing an immunoglobulin domain capable of binding to an antigen. An antibody can originate from any mammalian or avian species, e.g., human, rodent (e.g., mouse, rabbit), goat, chicken, etc., or can be generated using, e.g., phage display. The antibody may be a member of any immunoglobulin class, e.g., IgG, IgM, IgA, IgD, IgE, or subclasses thereof such as IgG1, IgG2, etc. In various embodiments of the invention “antibody” refers to an antibody fragment such as an Fab′, F(ab′)2, scFv (single-chain variable) or other fragment that retains an antigen binding site, or a recombinantly produced scFv fragment, including recombinantly produced fragments. An antibody can be monovalent, bivalent or multivalent in various embodiments. The antibody may be a chimeric or “humanized” antibody, which can be generated using methods known in the art. An antibody may be polyclonal or monoclonal, though monoclonal antibodies may be preferred. Methods for producing antibodies that specifically bind to virtually any molecule of interest are known in the art. In some aspects the antibody is an intrabody, which may be expressed intracellularly. In some embodiments a compound comprises a single-chain antibody and a protein transduction domain (e.g., as a fusion polypeptide).

Compounds to be screened can come from any source, e.g., natural product libraries, combinatorial libraries, libraries of compounds that have been approved by the FDA or another health regulatory agency for use in treating humans, etc. A library is often a collection of compounds that can be presented or displayed such that the compounds can be identified in a screening assay. In some embodiments compounds in the library are housed in individual wells (e.g., of microtiter plates), vessels, tubes, etc., to facilitate convenient transfer to individual wells or vessels for contacting cells, performing cell-free assays, etc. Numerous compound libraries are commercially available and can be used in the invention. The library may be composed of molecules having common structural features which differ in the number or type of group attached to the main structure or may be completely random. The method may encompass performing high througput screening. In some embodiments at least 100; 1,000; 10,000; 50,000; or 100,000 compounds are tested. Compounds identified as “hits” can then be tested in additional assays, e.g., to assess their effect on transcription, complex formation, cell proliferation, etc. Compounds identified as having a useful effect can be selected and systematically altered, e.g., using rational design, to optimize binding affinity, avidity, specificity, or other parameters. For example, one can screen a first library of compounds using the methods described herein, identify one or more compounds that are “hits” or “leads” (by virtue of, for example, their ability to inhibit metastasis), and subject those hits to systematic structural alteration to create a second library of compounds structurally related to the hit or lead. The second library can then be screened using the methods described herein or other methods known in the art. A compound can be modified or selected to achieve (i) improved potency, (ii) decreased toxicity and/or decreased side effects; (iii) modified onset of therapeutic action and/or duration of effect; and/or (iv) modified pharmacokinetic parameters (absorption, distribution, metabolism and/or excretion).

The invention encompasses the recognition that multiple histone deacetylase (HDAC) genes were identified as hits in the inventive shRNA screen described in more detail elsewhere herein (see Table S9, which lists mouse HDACs and identities those with a Z-score of greater than 1.5 (or less than −1.5). In another inventive shRNA screen using human rather than mouse ES cells, HDACs 5 and 6 were identified, Modulating HDAC activity is of use in certain embodiments of the invention to modulate function of a Cohesin-Mediator complex. For example, an HDAC could modify a Mediator or Cohesin component, thereby modulating function of the component and/or of a complex containing it. In some embodiments, a compound of interest herein comprises a histone deacetylase (HDAC) modulator. In some embodiments the HDAC modulator is an HDAC inhibitor. A wide variety of HDAC inhibitors are known in the art and can be used in the invention. Exemplary compounds are, e.g., phenylbutyric acid, valproic acid, and suberoylanilide hydroxamic acid (SAHA). One of skill in the art will be aware of many others. In some embodiments, the HDAC is HDAC 1, 2, 3, 5, 6, 7, 8, 9, 10, 11. In some embodiments, an HDAC inhibitor is contacted with a cell and a function of a Cohesin-Mediator complex is assessed.

The four proteins CDK8, cyclin C, Med12, and Med13 can associate with other Mediator components/complexes and are presumed to form a stable “subcomplex”. In certain embodiments, a compound of interest modulates a function of a complex comprising CDK8/cyclinC and, optionally, one or more Mediator components such as Med12 and/or Med 13. In some embodiments, a compound inhibits at least one subunit of a CDK8/cyclin/Med12/Med13 subcomplex. In some aspects, a compound of interest comprises a CDK8 inhibitor. A variety of compounds that inhibit CDK8 are known in the art and can be used in the invention. In some embodiments a CDK8 inhibitor comprises a truncated version of cyclin C. In some embodiments, flavopiridol or compound H7 or an analog thereof is used. See Rickert, P. et al. Oncogene 18: 1093-1102, 1999. In some embodiments, a compound inhibits expression of at least one subunit of a CDK8/cyclin/Med12/Med13 subcomplex. In some embodiments a compound inhibits formation of, or disrupts, a CDK8/cyclin/Med12/Med13 subcomplex. In various embodiments a compound that inhibits a CDK8/cyclin/Med12/Med13 subcomplex acts on the complex or component(s) thereof when the subcomplex is physically associated with the Mediator core and/or when the subcomplex or component(s) thereof are free in the cell and not associated with the Mediator core.

A subcomplex comprising CDK8/cyclin C (e.g., a CDK8/cyclin/Med12/Med13 subcomplex) may help maintain transcription at appropriate levels by at times limiting Mediator-dependent transcriptional activation of at least some genes. In some embodiments of the invention, Mediator function is increased by inhibiting a subcomplex comprising CDK8/cyclin C (e.g., a CDK8/cyclin/Med12/Med13 subcomplex). As described herein, a variety of diseases (Cohesin-associated disorders, sometimes termed “cohesinopathies”) are associated with mutations in genes encoding Cohesin components, in particular genes encoding Smc1a, Smc3, or Nipb1, which components are shown herein to be part of a transcription-specific Cohesin complex that interacts with Mediator. Partial loss of function of this transcription-specific Cohesin complex associated with mutations in the genes encoding Smc1a, Smc3, or Nipb1 is likely to be at least in part responsible for most cases of Cohesin-associated disorders, e.g., by reducing Cohesin-Mediator function that is needed for normal transcriptional activity. In some embodiments of the invention, a subcomplex comprising CDK8/cyclin C (e.g., a CDK8/cyclin/Med12/Med13 subcomplex) is inhibited in order to increase Mediator's transcriptional activation function, thereby at least in part compensating for reduced function of a transcription-specific Cohesin complex as occurs in certain Cohesin-associated disorders. Thus the invention provides a method of increasing Cohesin-Mediator function in a cell (e.g., in a cell in which such function is abnormally low, e.g., due to a mutation in a Cohesin component), the method comprising contacting the cell with an inhibitor of a subcomplex comprising CDK8/cyclin C, e.g., an inhibitor of a CDK8/cyclin C/Med12/Med13 complex. The invention further provides a method of treating a subject suffering from or at risk of a Cohesin-mediated disorder comprising administering an inhibitor of a subcomplex comprising CDK8/cyclin C, e.g., an inhibitor of a CDK8/cyclin C/Med12/Med13 complex, to the subject. In some embodiments the disorder is CdLS.

Compounds that modulate function of a Cohesin-Mediator complex and/or that modulate a Cohesin-Mediator interaction may be used in vitro or in vivo in an effective amount, e.g., an amount sufficient to achieve a biological response of interest. For example, an effective amount could be an amount that detectably modulates (a) binding of a Cohesin complex to Mediator complex or binding of a Cohesin component to a Mediator component; (b) occupancy of a cell type specific gene by Cohesin-Mediator complex; (c) expression or activity of a cell type specific gene; (d) response to a signal transduction pathway. In some embodiments, such modulation alters the binding, occupancy, expression, or response by a desired or predetermined amount. For example, the alteration (e.g., increase, decrease) can be by a factor of at least 1.5, 2, 5, 10, or more. In other embodiments, the alteration is by at least 10% of an original level, e.g., 10%, 25%, 50%, 75%, or more in various embodiments.

In some embodiments an effective amount reduces one or more symptoms or manifestations of a disorder, e.g., reduces the likelihood of recurrence or progression of a disorder, or reduces the extent to which a disorder manifests.

The compounds may be administered in a pharmaceutical composition. A pharmaceutical composition can comprise a variety of pharmaceutically acceptable carriers. Pharmaceutically acceptable carriers are well known in the art and include, for example, aqueous solutions such as water, 5% dextrose, or physiologically buffered saline or other solvents or vehicles such as glycols, glycerol, oils such as olive oil or injectable organic esters that are suitable for administration to a human or non-human subject. In some embodiments, a pharmaceutically acceptable carrier or composition is sterile. A pharmaceutical composition can comprise, in addition to the active agent, physiologically acceptable compounds that act, for example, as bulking agents, fillers, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients. The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition. Compounds can be present as salts in a composition. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore it will be appreciated that compounds may be modified, e.g., with targeting moieties, moieties that increase their uptake, biological half-life (e.g., pegylation), etc.

A pharmaceutical composition could be in the form of a liquid, gel, lotion, tablet, capsule, ointment, transdermal patch, etc. A pharmaceutical composition can be administered to a subject by various routes including, for example, parenteral administration. Exemplary routes of administration include intravenous administration; respiratory administration (e.g., by inhalation), nasal administration, intraperitoneal administration, oral administration, subcutaneous administration, intrasynovial administration, transdermal administration, and topical administration. For oral administration, the compounds can be formulated with pharmaceutically acceptable carriers as tablets, pills, dragees, capsules, liquids, gels, syrups, slurries, suspensions, etc. In some embodiments a compound may be administered directly to a tissue e.g., a tissue, e.g., in which cancer cells are or may be present or in which the cancer is likely to arise. Direct administration could be accomplished, e.g., by injection or by implanting a sustained release implant within the tissue. In some embodiments at least one of the compounds is administered by release from an implanted sustained release device, by osmotic pump or other drug delivery device. A sustained release implant could be implanted at any suitable site. In some embodiments, a sustained release implant may be particularly suitable for prophylactic treatment of subjects at risk of developing a recurrent cancer. In some embodiments, a sustained release implant delivers therapeutic levels of the active agent for at least 30 days, e.g., at least 60 days, e.g., up to 3 months, 6 months, or more. One skilled in the art would select an effective dose and administration regimen taking into consideration factors such as the patient's weight and general health, the particular condition being treated, etc. Exemplary doses may be selected using in vitro studies, tested in animal models, and/or in human clinical trials as standard in the art.

In some embodiments, a pharmaceutical composition is delivered by means of a microparticle or nanoparticle or a liposome or other delivery vehicle or matrix. A number of biocompatible synthetic or naturally occurring polymeric materials are known in the art to be of use for drug delivery purposes. Examples include polylactide-co-glycolide, polycaprolactone, polyanhydride, cellulose derivatives, and copolymers or blends thereof. Liposomes, for example, which consist of phospholipids or other lipids, are nontoxic, physiologically acceptable and metabolizable carriers that are relatively simple to make and administer.

Pharmaceutical compositions comprising a compound as described herein are an aspect of the invention. The pharmaceutical composition(s) may be packaged with a suitable label describing their use in a method of the invention (e.g., instructions for use to treat a disorder of interest).

Compounds useful treating a disease, e.g., a Cohesin-associated disease or a Mediator-associated disease, can be administered in combination with other compounds useful for treating the disease. See, e.g., Goodman & Gilman, supra; Katzung, supra. In some embodiments, a compound that modulates Cohesin-Mediator function is administered to a subject suffering from or at risk of a proliferative disorder, e.g., cancer, in combination with one or more other compounds useful for treating cancer, e.g., an approved chemotherapeutic agent or radiation therapy.

“Administered in combination” means that both compounds are administered to a subject. Such administration is sometimes referred to herein as coadministration. The compounds can be administered in the same composition or separately. When they are coadministered, the two may be given simultaneously or sequentially and in either instance, may be given separately or in the same composition, e.g., a unit dosage (which includes two or more compounds). The Cohesin-Mediator modulator can be given prior to or after administration of the second compound provided that they are given sufficiently close in time to have a desired effect, e.g., treating a disease. In some embodiments, administration in combination of first and second compounds is performed such that (i) a dose of the second compound is administered before more than 90% of the most recently administered dose of the first agent has been metabolized to an inactive form or excreted from the body; or (ii) doses of the first and second compound are administered within 48 hours of each other, or (iii) the agents are administered during overlapping time periods (e.g., by continuous or intermittent infusion); or (iv) any combination of the foregoing. Multiple compounds are considered to be administered in combination if the afore-mentioned criteria are met with respect to all compounds, or in some embodiments, if each compound can be considered a “second compound” with respect to at least one other compound of the combination. The compounds may, but need not be, administered together as components of a single composition. In some embodiments, they may be administered individually at substantially the same time (e.g., within less than 1, 2, 5, or 10 minutes of one another). In some embodiments they may be administered individually within a short time of one another (by which is meant less than 3 hours, sometimes less than 1 hour, sometimes within 10 or 30 minutes apart). The compounds may, but need not, be administered by the same route of administration.

Those skilled in the art will recognize, or be able to ascertain using no more than routine experimentation, many equivalents to the specific embodiments of the invention described herein. The scope of the present invention is not intended to be limited to the description or examples herein. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

The invention encompasses all variations, combinations, and permutations in which one or more limitations, elements, clauses, descriptive terms, etc., from one or more of the claims or from the description (including specific details in the experimental section) is introduced into another claim dependent on the same base claim (or, as relevant, any claim) unless otherwise indicated or unless it would be evident to one of ordinary skill in the art that a contradiction or inconsistency would arise, Embodiments and aspects of the invention may be freely combined unless inconsistent, contradictory, or mututally exclusive, Where lists or sets of elements are disclosed herein it is to be understood that each subgroup of the elements and each individual element are also disclosed. In general, where the invention, or aspects of the invention, is/are referred to as comprising particular elements, features, etc., certain embodiments of the invention or aspects of the invention consist, or consist essentially of, such elements, features, etc. It should also be understood that any embodiment of the invention can be explicitly excluded from the claims.

Where the description or claims recite a method, the invention encompasses inventive compositions used in performing the method, and products produced using the method. Where the description or claims recite a composition, the invention encompasses methods of using the composition and methods of making the composition. Any composition or method of the invention relating to a nucleic acid, protein, complex, cell, organ, tissue, disorder, cell type, cell state, or subject can include a step of identifying or selecting, such a nucleic acid, protein, complex, cell, organ, tissue, disorder, cell type, cell state, or subject, and/or a step of providing such a nucleic acid, protein, complex, cell, organ, tissue, or subject. One of ordinary skill in the art will appreciate that the phrase “of interest” as used herein, e.g., as in “cell state of interest” “disorder of interest” is used for convenience, is optional, and is not intended limit the invention.

Where ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a list of numerical values is stated herein (whether or not prefaced by “at least”), the invention includes embodiments that relate analogously to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

All patents, patent applications, publications, references, websites, databases, etc., cited in the instant patent application (including all portions thereof) are incorporated by reference in their entirety.

### EXAMPLES

### Example 1

### Mediator and Cohesin Contribute to ES Cell State

Transcription factors control the gene expression programs that establish and maintain cell state^{1,2}. These factors bind to enhancer elements that can be located some distance from the core promoter elements where the transcription initiation apparatus is bound^{3,4}. The enhancer-bound transcription factors bind coactivators such as mediator and p300, which in turn bind the transcription initiation apparatus⁵⁻⁹. This set of interactions, well established in vitro, implies that activation of gene expression is accompanied by DNA loop formation. Indeed, chromosome conformation capture (3C) experiments have confirmed that some enhancers are brought into proximity of the promoter during active transcription¹⁰⁻¹². If DNA looping does occur between the enhancers and core promoters of active genes, we reasoned that it would be valuable to identify the proteins that have key roles in the formation and stability of such loops.

We used a small hairpin RNA (shRNA) library to screen for regulators of transcription and chromatin necessary for the maintenance of murine embryonic stem (ES) cell state (Supplementary FIG. 1a, b). The screen was designed to detect changes in the level of the ES cell transcription factor Oct4, a master regulator of the pluripotent state, in cells that remain viable during the course of the experiment. Most known regulators of ES cell state were identified in this screen, including Oct4, Sox2, Nanog, Esrrb, Sal14 and Stat3 (FIG. 1a and Supplementary Tables 1, 2), indicating that other components identified in this screen may also be important for maintenance of ES cell state. It was particularly striking that many of the subunits of the mediator complex (Med6, Med7, Med10, Med12, Med14, Med15, Med17, Med21, Med24, Med27, Med28 and Med30), the cohesin complex (Smc1a, Smc3 and Stag2) and the cohesin loading factor Nipb1 emerged from the screen. Mediator, cohesin and Nipb1 are thought to have essential roles in gene expression and chromosome segregation^{5-9,13-15}, so their identification in this screen indicates that ES cell state may be highly sensitive to a reduction in the levels of these protein complexes.

The loss of ES cell state is characterized by reduced levels of Oct4 protein, a loss of ES cell colony morphology, reduced levels of mRNAs specifying transcription factors associated with ES cell pluripotency (for example, Oct4, Sox2 and Nanog) and increased expression of mRNAs encoding developmentally important transcription factors^{16,17}. We confirmed that shRNAs targeting mediator, cohesin and Nipb1 produced all these effects (FIG. 1b, c, Supplementary Table 3 and Supplementary FIGS. 1c-f and 2). Thus, reduced levels of mediator, cohesin and Nipb1 have the same effect on these key characteristics of ES cell state as loss of Oct4 itself.

### Example 2

### Mediator Occupies Enhancers and Promoters

Transcription factors bound to enhancers bind coactivators such as the mediator complex, which in turn can recruit RNA polymerase II to the core promoter⁵⁻⁹. It has not been clear, however, how often mediator is employed as a coactivator at active genes in vivo. We used chromatin immunoprecipitation coupled with massively parallel DNA sequencing (ChIP-Seq) to identify sites occupied by mediator subunits Med1 and Med12 in the ES cell genome (FIG. 2, Supplementary FIG. 3 and Supplementary Tables 4-6). Med1 and Med12 were studied because they occupy different functional domains within the mediator complex¹⁸. Analysis of the results revealed that mediator occupied the promoter regions of at least 60% of actively transcribed genes (Supplementary FIG. 4).

More detailed examination of the ChIP-Seq data for mediator with that of key transcription factors (Oct4, Nanog and Sox2) and components of the transcription initiation apparatus (RNA polymerase II (Pol2) and TATA-binding protein (TBP)) revealed that mediator is found at both the enhancers and core promoters of actively transcribed genes (FIG. 2a). For example, mediator was detected at the well-characterized enhancers of the Oct4 (also called Pou5f1) and Nanog genes¹⁹⁻²¹, which are bound by the ES cell master transcription factors Oct4, Sox2 and Nanog^{22,23}. Mediator was also detected at the Oct4 and Nanog core promoters together with Pol2 and TBP. These observations provide in vivo support for the model that mediator bridges interactions between transcription factors at enhancers and the transcription initiation apparatus at core promoters.

### Example 3

### Mediator and Cohesin Co-Occupy Active Genes

Cohesin has been shown to occupy sites bound by CCCTC-binding factor (CTCF) and to contribute to DNA loop formation associated with gene repression or activation²⁴⁻²⁶. Cohesin has also been demonstrated to occupy sites independently of CTCF, but the role of cohesin at these sites is not known²⁷. We used ChIP-Seq to determine the genome-wide occupancy of the two cohesin core complex proteins, Smc1a and Smc3, the knockdown of which resulted in a loss of Oct4 (FIG. 2, Supplementary FIG. 3 and Supplementary Tables 4-6). The results show that cohesin occupies sites bound by CTCF, as expected, but also occupies the enhancer and core promoter sites bound by mediator (FIG. 2a, b and Supplementary FIG. 5). The regions co-occupied by cohesin and mediator were associated with RNA polymerase II whereas those co-occupied by cohesin and CTCF were not (FIG. 2c). These results demonstrate that there is a population of cohesin that is associated with the enhancer and core promoter sites occupied by mediator in many active promoters of ES cells.

The cohesin loading factor Nipb1, which was also identified in the shRNA screen, has been implicated in transcriptional regulation and is mutated in the majority of individuals afflicted with Cornelia de Lange syndrome, a developmental disorder^{14,28,29,50}. Surprisingly, ChIP-Seq data revealed that Nipb1 generally occupies the enhancer and core promoter regions bound by mediator and cohesin, but is rarely found at CTCF and cohesin co-occupied sites (FIG. 2a-c and Supplementary FIG. 5). The association between Nipb1 and mediator-cohesin sites was highly significant (P<10⁻³⁰⁰) whereas the association of Nipb1 with CTCF-cohesin sites was no greater than expected by chance (P=1). Thus, the cohesin loading factor Nipb1 is associated with cohesin-mediator sites but not with cohesin-CTCF sites in ES cells. These results link Nipb1 and Cornelia de Lange syndrome to a form of cohesin associated with mediator at actively transcribed genes.

The co-occupancy of mediator, cohesin and Nipb1 at the promoter regions of Oct4 and other active ES cell genes (FIG. 2a, c) indicates that these complexes may all contribute to the control of transcription. If mediator, cohesin and Nipb1 function together to regulate the genes they occupy, then we would expect that knockdown of Nipb1 or key components of the mediator or cohesin complexes would have similar effects on expression of these genes. Analysis of changes in mRNA levels in knockdown cells revealed that this is the case (FIG. 2d). Of the approximately 2,700 genes that are co-occupied by mediator, cohesin, Nipb1 and Pol2 at high confidence, approximately 700 showed significant expression changes (P<0.01) in each of the mediator, cohesin and Nipb1 knockdown data sets (FIG. 2d and Supplementary Table 3). The three knockdowns had markedly similar effects at this set of genes, which may explain why mediator, cohesin and Nipb1 knockdowns cause very similar ES cell phenotypes (Supplementary FIG. 6). These results indicate that actively transcribed genes occupied by mediator, cohesin and Nipb1 typically depend on each of these factors for normal expression.

### Example 4

### Mediator and Cohesin Interact

The ChIP-Seq results show that mediator, cohesin and Nipb1 co-occupy thousands of sites in the ES cell genome and thus indicate that these complexes may physically interact. To investigate this possibility, we crosslinked ES cells using the ChIP protocol, immunoprecipitated complexes using antibodies against mediator (Med1, Med12) and cohesin (Smc1a, Smc3) and determined whether the mediator subunit Med23 could be detected in the immunoprecipitate (FIG. 3a). The results showed that mediator and cohesin components can co-precipitate with one another. Furthermore, an antibody against Nipb1 co-precipitated both cohesin and mediator subunits (FIG. 3b). These results suggest that mediator, cohesin and Nipb1 interact.

If mediator and cohesin do indeed interact, then they should co-purify. Mediator was affinity purified from ES cell nuclei using a multi-step approach (FIG. 3c). First, the activation domain of SREBP-1a, which is known to bind mediator, was used for an initial affinity purification step^{30,31}. After a series of high-salt washes, hound proteins were eluted and subjected to a second orthogonal immunoprecipitation step, with an anti-CDK8 antibody resin. CDK8 is a mediator-specific subunit, which ensured that mediator and mediator-associated factors would be specifically retained on this antibody column. After binding, the CDK8 antibody resin was subjected to a series of high-salt washes, and bound proteins were then eluted and examined by silver stain and western blot analysis. The results show that cohesin and Nipb1 co-purified with mediator throughout this protocol (FIG. 3c). Additional evidence for a mediator-cohesin interaction came from an unbiased, multidimensional protein identification technology (MudPIT)-based screen for mediator-associated factors in HeLa cells³². Collectively, these results indicate that mediator, cohesin and Nipb1 physically interact and suggest that this interaction accounts for their co-occupancy at active promoters in vivo.

### Example 5

### Mediator and Cohesin Predict DNA Looping

Our evidence shows that mediator, cohesin and Nipb1 interact and co-occupy the enhancer and core promoter regions of a set of active genes in ES cells, indicating that they contribute to DNA looping between the enhancer and core promoter of these genes. We selected four different loci, Nanog, Phc1, Oct4 and Lefty1, to test enhancer-promoter interaction frequencies in ES cells and in murine embryonic fibroblasts (MEFs). These genes were selected because mediator and cohesin occupy their enhancer and core promoter regions in ES cells, where they have a positive role in their transcription, whereas mediator and cohesin are not present at these genes in MEFs, where these genes are transcriptionally silent.

We used 3C technology³³ to determine whether a looping event could be detected between the enhancer and promoter of Nanog, Phc1, Oct4 and Lefty1 loci in both ES cells and MEFs (FIG. 4 and Supplementary FIG. 7). For all loci tested we observed an increased interaction frequency between the core promoter and the enhancer in ES cells, indicating the presence of a DNA loop. Importantly, this interaction was not observed in MEFs where Nanog, Phc1, Oct4 and Lefty1 are silent and not occupied by mediator and cohesin. Furthermore, a reduction in Smc1a or Med12 expression levels resulted in a decreased interaction frequency between the core promoter and enhancer of Nanog (Supplementary FIG. 8). These 3C results are consistent with a model where the mediator-cohesin-Nipb1 complex promotes cell-type-specific gene activation through enhancer-promoter DNA looping.

### Example 6

### Mediator and Cohesin Occupy Cell-Type Specific Genes

The observation that mediator, cohesin and Nipb1 occupied the promoters of ES-cell-specific genes such as those encoding the pluripotency regulators Oct4 and Nanog (FIG. 2a) led us to ask whether mediator and cohesin tend to occupy cell-type-specific genes. Indeed, mediator and cohesin were found to co-occupy very different sets of promoters in ES cells and MEFs (FIG. 5a and Supplementary Tables 4-6). In contrast, many of the sites occupied by cohesin and CTCF in ES cells were also co-occupied by these proteins in MEFs (FIG. 5b and Supplementary Tables 4-6). The levels of mediator were found to be considerably higher in ES cells than in MEFs (FIG. 5c), accounting for the differences in the number of sites co-occupied by mediator and cohesin in the two cell types. These observations indicate that mediator and cohesin have especially important roles in cell-type-specific gene expression and thus, in cell-type-specific chromosome structure.

Discussion

Evidence for specific DNA loop formation during transcription initiation was first described in bacteria and bacteriophage gene expression systems³⁴⁻³⁹. For example, bacterial DNA-binding factors can bind elements located upstream of sites occupied by sigma-54 RNA polymerases and cause looping of the intervening DNA when the transcription factors bind to polymerase. Proteins that act to stabilize these DNA loops and thus contribute to gene activity were also identified in these systems⁴⁰⁻⁴². Our results suggest a similar model for the contributions of mediator and cohesin to gene regulation and DNA looping in vertebrate cells. In this model, DNA loop formation between enhancers and core promoters occurs as a consequence of the interaction between enhancer-bound transcription activators, mediator and promoter-bound RNA polymerase II. When the transcription activators bind mediator, the mediator complex undergoes a conformational change^{32,43}, and this activator-bound form of mediator binds cohesin and its loading factor Nipb1, which all contribute to gene activity.

Through their roles in DNA loop formation at a subset of active promoters, mediator, cohesin and Nipb1 link gene expression with cell-type-specific chromatin structure. In this context, we note that mutations in the genes encoding mediator and cohesin components and Nipb1 can cause an array of human developmental syndromes and diseases. Mediator mutations have been associated with Opitz-Kaveggia (FG) syndrome, Lujan syndrome and schizophrenia⁴⁴⁻⁴⁷. Mutations in Nipb1 are responsible for most cases of Cornelia de Lange syndrome, which is characterized by developmental defects and mental retardation and seems to be the result of mis-regulation of gene expression rather than chromosome cohesion or mitotic abnormalities^{28,29,48}. We suggest that these disorders and diseases are due to deficiencies in the chromatin structure generated by mediator and cohesin, which we have shown is essential for normal transcriptional programs in ES cells.

Methods Summary

High-Throughput shRNA Screening

High-throughput RNAi screening was performed at the Broad Institute RNAi Platform. Murine ES cells were seeded in 384-well plates, infected with an individual lentiviral shRNA construct, treated with puromycin, and crosslinked with 4% paraformaldehyde 5 days after infection. Cells were stained with Hoechst and for Oct4 and imaged with an ArrayScan HCS Reader (Cellomics). Cells were identified with Cellomics software, the average Oct4 pixel intensity was quantified and an average was calculated for all cells identified in the well.

ChIP-Seq

Chromatin immunoprecipitations (ChIPs) were performed and analysed as previously described⁴⁹. ChIP-Seq and microarray data have been deposited in the Gene Expression Omnibus under accession code GSE22557.

Microarray Analysis

Expression analyses were carried out with Agilent DNA microarrays using labelled cRNA generated from shRNA GFP (control), Smc1a, Med12 and Nipb1 infected murine ES cells.

Mediator Complex Purification

The mediator complex was purified from murine ES cell nuclear extracts, essentially as described³².

Chromosome Conformation Capture (3C)

Murine ES cells or MEFs were crosslinked, lysed and chromatin was digested with 1,000 units HaeIII or 2,000 units MspI. Crosslinked fragments were ligated with 50 units T4 DNA ligase for 4 h at 16° C. 3C product detection was done in triplicate by qPCR and averaged for each primer pair. Each data point was first corrected for PCR bias by dividing the average of three PCR signals by the average signal in the BAC control template. Data from ES cells and MEFs were normalized to each other using the interaction frequencies between fragments in control regions. 3C primer sequences are listed in Supplementary Table S7.

### REFERENCES

1. Ptashne, M. & Gann, A. Genes and Signals 1st edn (Cold Spring Harbor Laboratory Press, 2002),
2. Graf, T. & Enver, T. Forcing cells to change lineages. Nature 462, 587-594 (2009).
3. Panne, D. The enhanceosome. Curr. Opin. Struct. Biol. 18, 236-242 (2008).
4. Bulger, M. & Groudine, M. Enhancers: the abundance and function of regulatory sequences beyond promoters. Dev. Biol. 339, 250-257 (2010).
5. Roeder, R. G. Role of general and gene-specific cofactors in the regulation of eukaryotic transcription. Cold Spring Harb. Symp. Quant. Biol. 63, 201-218 (1998).
6. Malik, S. & Roeder, R. G. Dynamic regulation of pol II transcription by the mammalian Mediator complex. Trends Biochem. Sci. 30, 256-263 (2005).
7. Kornberg, R. D. Mediator and the mechanism of transcriptional activation. Trends Biochem. Sci. 30, 235-239 (2005).
8. Conaway, R. C., Sato, S., Tomomori-Sato, C., Yao, T. & Conaway, J. W. The mammalian Mediator complex and its role in transcriptional regulation. Trends Biochem. Sci. 30, 250-255 (2005).
9. Taatjes, D. J. The human Mediator complex: a versatile, genome-wide regulator of transcription. Trends Biochem. Sci. 35, 315-322 (2010),
10, Vakoc, C. R. et al. Proximity among distant regulatory elements at the □-globin locus requires GATA-1 and FOG-1. Mol. Cell. 17, 453-462 (2005).
11. Jiang, H. & Peterlin, B. M. Differential chromatin looping regulates CD4 expression in immature thymocytes. Mol. Cell. Biol. 28, 907-912 (2008).
12. Miele, A. & Dekker, J. Long-range chromosomal interactions and gene regulation. Mol. Biosyst. 4, 1046-1057 (2008).
13. Nasmyth, K. & Haering, C. H. Cohesin: its roles and mechanisms. Annu. Rev, Genet, 43, 525-558 (2009).
14. Liu, J. et al. Transcriptional dysregulation in NIPBL and cohesin mutant human cells. PLoS Blot, 7, e1000119 (2009).
15. Wood, A. J., Severson, A. F. & Meyer, B. J. Condensin and cohesin complexity: the expanding repertoire of functions. Nature Rev. Genet. 11, 391-404 (2010).
16. Niwa, H., Miyazaki, J. & Smith, A. G. Quantitative expression of Oct-3/4 defines differentiation, dedifferentiation or self-renewal of ES cells. Nature Genet. 24, 372-376 (2000).
17. Jaenisch, R. & Young, R. Stem cells, the molecular circuitry of pluripotency and nuclear reprogramming. Cell 132, 567-582 (2008).
18. Knuesel, M. T., Meyer, K. D., Bernecky, C. & Taatjes, D. J. The human CDK8 subcomplex is a molecular switch that controls Mediator coactivator function. Genes Dev. 23, 439-451 (2009).
19. Yeom, Y. I. et al. Germline regulatory element of Oct-4 specific for the totipotent cycle of embryonal cells. Development 122, 881-894 (1996).
20. Okumura-Nakanishi, S., Saito, M., Niwa, H. & Ishikawa, F. Oct-3/4 and Sox2 regulate Oct-3/4 gene in embryonic stem cells. J. Biol. Chem., 280, 5307-5317 (2005).
21. Wu, Q. et al. Sal14 interacts with Nanog and co-occupies Nanog genomic sites in embryonic stem cells. J. Biol. Chem. 281, 24090-24094 (2006).
22. Boyer, L. A. et al. Core transcriptional regulatory circuitry in human embryonic stem cells, Cell 122, 947-956 (2005).
23. Loh, Y. H, et al. The Oct4 and Nanog transcription network regulates pluripotency in mouse embryonic stem cells. Nature Genet. 38, 431-440 (2006).
24. Wendt, K. S. et al. Cohesin mediates transcriptional insulation by CCCTC-binding factor, Nature 451, 796-801 (2008).
25. Hadjur, S. et al. Cohesins form chromosomal cis-interactions at the developmentally regulated IFNG locus. Nature 460, 410-413 (2009).</jrn>
26. Bose, T. & Gerton, J. L. Cohesinopathies, gene expression, and chromatin organization. J. Cell Biol. 189, 201-210 (2010).
27. Schmidt, D. et al. A CTCF-independent role for cohesin in tissue-specific transcription. Genome Res. 20, 578-588 (2010).
28. Tonkin, E. T., Wang, T. J., Lisgo, S., Bamshad, M. J. & Strachan, T. NIPBL, encoding a homolog of fungal Scc2-type sister chromatid cohesion proteins and fly Nipped-B, is mutated in Cornelia de Lange syndrome. Nature Genet. 36, 636-641 (2004).
29. Krantz, I. D, et al. Cornelia de Lange syndrome is caused by mutations in NIPBL, the human homolog of Drosophila melanogaster Nipped-B. Nature Genet. 36, 631-635 (2004).
30. Toth, J. I., Datta, S., Athanikar, J. N., Freedman, L. P. & Osborne, T. F. Selective coactivator interactions in gene activation by SREBP-1a and -1c. Mol. Cell. Biol. 24, 8288-8300 (2004).
31. Yang, F. et al. An ARC/Mediator subunit required for SREBP control of cholesterol and lipid homeostasis. Nature 442, 700-704 (2006).
32. Ebmeier, C. C. & Taatjes, D. J. Activator-Mediator binding regulates Mediator-cofactor interactions, Proc. Natl. Acad. Sci. USA 107, 11283-11288 (2010).</bok>
33. Dekker, J., Rippe, K., Dekker, M. & Kleckner, N. Capturing chromosome conformation. Science 295, 1306-1311 (2002). </jrn>
34. Ptashne, M. Gene regulation by proteins acting nearby and at a distance. Nature 322, 697-701 (1986).
35. Adhya, S. Multipartite genetic control elements: communication by DNA loop. Annu. Rev. Genet. 23, 227-250 (1989).
36. Schleif, R. DNA looping. Annu. Rev. Biochem. 61, 199-223 (1992).
37. Matthews, K. S. DNA looping. Microbiol. Rev. 56, 123-136 (1992).</jrn>
38. Bulger, M. & Groudine, M. Looping versus linking: toward a model for long-distance gene activation. Genes Dev. 13, 2465-2477 (1999).
39. Saiz, L. & Vilar, J. M. DNA looping: the consequences and its control. Curr. Opin. Struct. Biol. 16, 344-350 (2006).
40. Hoover, T. R., Santero, E., Porter, S. & Kustu, S. The integration host factor stimulates interaction of RNA polymerase with NIFA, the transcriptional activator for nitrogen fixation operons. Cell 63, 11-22 (1990).
41. Clayerie-Martin, F. & Magasanik, B. Role of integration host factor in the regulation of the glnHp2 promoter of Escherichia coli. Proc. Natl Acad. Sci. USA 88, 1631-1635 (1991).
42, Luijsterburg, M. S., White, M. F., van Driel, R. & Dame, R. T. The major architects of chromatin: architectural proteins in bacteria, archaea and eukaryotes. Crit. Rev. Biochem. Mol. Biol. 43, 393-418 (2008).
43. Taatjes, D. J., Naar, A. M., Andel, F. III, Nogales, E. & Tjian, R. Structure, function, and activator-induced conformations of the CRSP coactivator. Science 295, 1058-1062 (2002).
44. Philibert, R. A. & Madan, A. Role of MED12 in transcription and human behavior. Pharmacogenomics 8, 909-916 (2007).
45. Risheg, H. et al. A recurrent mutation in MED12 leading to R961W causes Opitz-Kaveggia syndrome. Nature Genet. 39, 451-453 (2007).
46. Schwartz, C. E. et al. The original Lujan syndrome family has a novel missense mutation (p.N1007S) in the MED12 gene. J. Med. Genet. 44, 472-477 (2007).
47. Ding, N. et al. Mediator links epigenetic silencing of neuronal gene expression with x-linked mental retardation. Mol. Cell 31, 347-359 (2008).
48. Strachan, T. Cornelia de Lange Syndrome and the link between chromosomal function, DNA repair and developmental gene regulation. Curr. Opin. Genet. Dev. 15, 258-264 (2005).
49. Marson, A. et al. Connecting microRNA genes to the core transcriptional regulatory circuitry of embryonic stem cells. Cell 134, 521-533 (2008).
50. Dorsett, D. Roles of the sister chromatid cohesion apparatus in gene expression, development, and human syndromes. Chromosoma 116, 1-13 (2007)

List of Tables Referred to in Examples 1-5

Supplementary Table 1—Z-scores of shRNAs Used in the Screen

Supplementary Table 2—Classification of Screen Hits

Supplementary Table 3—Med12, Smc1a and Nipb1 Knockdown Expression

Data

Supplementary Table 4—Bound Genomic Regions

Supplementary Table 5—Summary of Occupied Genes

Supplementary Table 6—Summary of ChIP-Seq Data Used

Supplementary Table 7—Chromosome Conformation Capture (3C) Primers

Table S8—Primers Used for Gene-Specific Chips

Note: Supplementary Tables 1, 3, 4, and 5 are available on the Nature website (http://www.nature.com) as Supplementary Tables for Kagey, M., et al., Mediator and cohesin connect gene expression and chromatin architecture. Nature. (2010) Sep. 23; 467(7314):430-5. Epub 2010 Aug. 18. (http://www.nature.com/nature/journal/v467/n7314/full/nature09380.html#/supplementary-information). The entire contents of Kagey, M., et al., Mediator and cohesin connect gene expression and chromatin architecture. Nature. (2010) Sep. 23; 467(7314); 430-5. Epub 2010 Aug. 18, including all Supplementary Information, Supplementary Tables, Supplementary Data, Supplementary Figures, is incorporated by reference herein.

Supplementary Data File 1

Formatted (.WIG) files for Med1_mES, Med12_mES, Nipb1_mES, Smc1a_mES, Smc3_mES, TBP_mES, Oct4_mES, Sox2_mES, Nanog_mES, Pol2_mES, H3K79me2_mES, CTCF_mES, Med1_MEFs, Med12_MEFs, Smc1a_MEFs and CTCF_MEFs.

Supplementary Data File 1 contains data zipped, formatted (WIG.GZ) for upload into the UCSC genome browser⁶. To upload the file, first unzip the files onto a computer with Internet access. Then use a web browser to go to http://genome.ucsc.edu/egi-bin/hgCustom?hgsid=105256378. Select genome (Mouse) and assembly (February 2006 (NCBI36/mm8)). In the “Paste URLs or Data” section, select “Browse . . . ” on the right of the screen. Use the pop-up window to select the unzipped files, and then select “Submit”. The upload process may take some time.

These files present ChIP-Seq data. The first track for each data set contains the ChIP-Seq density across the genome in 25 bp bins. The minimum ChIP-Seq density shown in these files is 0.5 reads per million. Subsequent tracks identify genomic regions identified as enriched (P-val<10⁻⁹).

This data is contained in 3 separate zipped files—see Supplementary Data 1—parts 1, 2 and 3 are available on the Nature website (http://www.nature.com) as Supplementary Data for Kagey, M., et al., Mediator and cohesin connect gene expression and chromatin architecture. Nature. (2010) Sep. 23; 467(7314):430-5. Epub 2010 Aug. 18. (http://www.nature.com/nature/journal/v467/n7314/full/nature09380.html#/supplementary-information).

Listing of Detailed Experimental Procedures

Cell Culture Conditions

Embryonic Stem Cells Mouse Embryonic Fibroblasts (MEFs)

High-Throughput shRNA Screening

Library Design and Lentiviral Production

Lentiviral Infections

Immunofluorescence

Image Acquisition and Analysis

Combining Screening Data (Supplementary Table 1) Criteria for Identifying

Screening Hits (Supplementary Table 2)

Validation of shRNAs

Lentiviral Production and Infection

Immunofluorescence

RNA Extraction, cDNA, and TaqMan Expression Analysis

Chromatin Immunoprecipitation ChIP-Seq Sample Preparation and Analysis

Sample Preparation

Polony Generation and Sequencing

ChIP-Seq Data Analysis

ChIP-Seq Density Map (Supplementary FIG. 4)

ChIP-Seq Enriched Region Maps (FIG. 2c and FIG. 5a, b)

Assigning ChIP-Seq Enriched Regions to Genes (Supplementary Table 5)

Note Regarding Summary of Occupied Genes Table (Supplementary Table 5)

Note Regarding Calculation of Co-occupied Regions (Supplementary Table 4)

Gene Specific ChIPs ChIP-Western and Co-Immunoprecipitation (FIG. 3a, b) Protein Extraction and Western Blot Analysis (FIG. 5c and Supplementary FIG. 3a) Mediator Affinity Purification Chromosome Conformation Capture (3C) Microarray Analysis

Cell Culture and RNA Isolation

Microarray Hybridization and Analysis

Determining Genes Co-occupied by Smc1a, Med12 and Nipb1 with Expression Changes (FIG. 2d)

Detailed Experimental Procedures

Cell Culture Conditions

Embryonic Stem Cells

V6.5 murine embryonic stem (mES) cells were grown on irradiated murine embryonic fibroblasts (MEFs) unless otherwise stated. Cells were grown under standard mES cell conditions as described previously⁷. Briefly, cells were grown on 0.2% gelatinized (Sigma, G1890) tissue culture plates in ESC media; DMEMKO (Invitrogen, 10829-018) supplemented with 15% fetal bovine serum (Hyclone,

characterized SH3007103), 1000 U/mL LIF (ESGRO, ESG1106), 100 μM nonessential amino acids (Invitrogen, 11140-050), 2 mM L-glutamine (Invitrogen, 25030-081), 100 U/mL penicillin, 100 μg/mL streptomycin (Invitrogen, 15140-122), and 8 mL/mL of 2-mercaptoethanol (Sigma, M7522).

Mouse Embryonic Fibroblasts (MEFs)

Low passage MEFs were grown on tissue culture plates in DMEM (Invitrogen, 11965) supplemented with 10% fetal bovine serum (Hyclone, characterized SH3007103), 100 μM nonessential amino acids (Invitrogen, 11140-050), 2 mM L-glutamine (Invitrogen, 25030-081), 100 U/mL penicillin, 100 μg/mL streptomycin (Invitrogen, 15140-122), and 8 nL/mL of 2-mercaptoethanol (Sigma, M7522).

High-Throughput shRNA Screening

Library Design and Lentiviral Production

Small hairpins targeting 197 chromatin regulators and 2021 transcription factors were designed and cloned into pLKO.1 lentiviral vectors (Open Biosystems) as previously described⁸. On average 5 different shRNAs targeting each chromatin regulator or transcription factor were used. Lentiviral supernatants were arrayed in 384-well plates with negative control lentivirus (shRNAs targeting GFP, RFP, Luciferase and LacZ)⁸.

Lentiviral Infections

Murine ES cells were split off MEFs and placed in a tissue culture dish for 45 minutes to selectively remove the MEFs. Murine ES cells were counted with a Coulter Counter (Beckman, #1499) and seeded using a μFill (Bioteck) at a density of 1500 cells/well in 384-well plates (Costar 3712) treated with 0.2% gelatin (Sigma, G1890). An initial cell plating density of 1500 cells/well was established so that an adequate amount of cells would survive puromycin selection for analysis. However, the initial cell plating density was kept low enough to avoid wells reaching confluency during the timeframe of the assay. One day following cell plating the media was removed, replaced with ESC media containing 8 μg/ml of polybrene (Sigma, H9268-10G) and cells were infected with 2 μl of shRNA lentiviral supernatant. Infections were performed in duplicate (transcription factor set) or quadruplicate (chromatin regulator set) on separate plates. Supplementary Table 1 denotes which screening set the shRNAs were in. Control wells on each plate were mock infected and designated as “Empty”.

Positive control wells on each plate were infected with 3 μl of validated control shRNA lentiviral supernatant targeting Oct4 (TRCN0000009613), Tcf3 (TRCN0000095454) and Stat3 (TRCN0000071454) that was generated independently of the screening sets (Lentiviral Production and Infection). Sequence and shRNAs are available from Open Biosystems. Plates were spun for 30 minutes at 2150 rpm following infection. Twenty-four hours post infection cells were treated with 3.5 μg/ml of puromycin (Sigma, P8833) in ESC media to select for stable integration of the shRNA construct. ESC media with puromycin

was changed daily. Five days post infection cells were crosslinked for 15 minutes with 4% paraformaldehyde (EMS Diasum, 15710).

Immunofluorescence

Following crosslinking, the cells were washed once with PBS, twice with blocking buffer (PBS with 0.25% BSA, Sigma, A3059-10G) and then permeabilized for 15 minutes with 0.2% Triton X-100 (Sigma, T8797-100 ml). After two washes with blocking buffer cells were stained overnight at 4° C. for Oct4 (Santa Cruz Biotechnology, sc-5279; 1:100 dilution) and washed twice with blocking buffer. Cells were incubated for 4 hours at room temperature with goat anti-mouseconjugated Alexa Fluor 488 (Invitrogen; 1:200 dilution) and Hoechst 33342 (Invitrogen; 1:1000 dilution), Finally, cells were washed twice with blocking buffer and twice with PBS before imaging.

Image Acquisition and Analysis

Image acquisition and data analysis were performed essentially as described⁸. Stained cells were imaged on an Arrayscan HCS Reader (Cellomics) using the standard acquisition camera mode (10× objective, 9 fields). Hoechst was used as the focus channel. Objects selected for analysis were identified based on the Hoechst staining intensity using the Target Activation Protocol and the Fixed Threshold Method. Parameters were established requiring that individual objects pass an intensity and size threshold. The Object Segmentation Assay Parameter was adjusted for maximal resolution between individual cells. Following object selection, the average Oct4 pixel staining intensity was determined per object and then a mean value for each well was calculated. Image acquisition for a well continued until at least 2500 objects were identified, the entire well (9 fields) was imaged or less than 20 objects were identified for three fields imaged in a row. To account for viability defects or low titer lentivirus for the chromatin regulator screening set an shRNA was excluded from subsequent analysis if less than 250 objects were identified for any one of the 4 replicates. The 250 identified objects threshold was determined based on the average number of identified objects for the “Empty” (no virus) wells (mean: 53.4, standard deviation: 49.3). To account for viability defects or low titer lentivirus for the transcription factor screening set a shRNA was excluded from subsequent analysis if less than 300 objects were identified for any one of the 2 replicates. The 300 identified objects threshold was determined based on the average number of identified objects for the “Empty” (no virus) wells (mean: 39.2, standard deviation: 147.5).

To normalize for plate effects, a Z-score based on the Oct4 staining intensity was calculated for each well using the following negative control infections, 24 different shRNAs targeting GFP, 16 different shRNAs targeting RFP, 25 different shRNAs targeting Luciferase and 20 different shRNAs targeting LacZ. There were a total of between 16 and 22 wells infected with various negative control shRNAs on each 384-well plate, with the exception of one plate within the transcription factor set that contained 99 wells with control infections. The average Oct4 staining intensity for the negative control infected wells was calculated along with a standard deviation to give an estimation of the amount of the signal variability. The average Oct4 staining intensity for all the negative control infected wells on a plate and the standard deviation were utilized to calculated a Z-score for every well on the plate. The Z-scores for the four quadruplicate infections (chromatin regulator set) or two duplicate infections (transcription factor set) were averaged for a final Z-score for every shRNA. The Z-score data for both sets were combined (Supplementary Table 1). Representative control 384-well plate images (shRNAs targeting Oct4, Stat3, Tcf3 and GFP) were exported (Cellomics Software), converted from DIBs to TIFs (CellProfiler, http://www.cellprofiler.org), and manipulated with Photoshop CS3 Extended (Supplementary FIG. 1a, b).

Combining Screening Data (Supplementary Table 1)

We recently published the results of an ES screen where 197 chromatin regulators were selectively targeted for knockdown⁹. For the present study we screened an additional 2021 genes primary encoding transcription factors. In order to generate a more complete picture of factors required for maintaining ES cell state we included the set of chromatin regulator results from the previous study. The shRNAs from each set are denoted in Supplementary Table 1.

The same methodology was followed for screening with both the chromatin regulator and transcription factor sets with the following exception, infections for the chromatin regulator set were done in quadruplicate and infections for the transcription factor set were carried out in duplicate, due to the large size of the transcription factor screening set (30×384-well plates, 2021 genes). Because the average Z-scores of the added controls (Oct4 and Stat3) were within close proximity for both screening sets (Chromatin Regulator Set: −3.3 and −2.4 for Oct4 and Stat3 respectively; Transcription Factor Set: −3.0 and −2.1 for Oct4 and Stat3 respectively) we reasoned that Z-scores between the two screening sets were comparable.

Criteria for Identifying Screening Hits (Supplementary Table 2)

We used multiple Z-score level thresholds to select chromatin regulators and transcription factors that had significantly reduced Oct4 levels for inclusion in Supplementary Table 2. First, a chromatin regulator or transcription factor had to have at least two shRNA with a Z-score less than −1.5 and it was possible to classify the gene based on the literature. Second, a chromatin regulator or transcription factor with a single shRNA hit and a Z-score of less than −1.5 was also included if it could be classified with one of the multiple shRNA hits. Third, the following chromatin regulators (Cbx7, Cbx8/Pc3 and Ezh2) were included even though each was only a single shRNA hit, because all had strong negative Z-scores, all are polycomb proteins, and polycomb has been previously demonstrated to be important for regulating ES cell state¹⁰. The −1.5 cut-off was chosen because it was within close proximity to the Z-score of the Stat3 controls (−2.4 and −2.1 for the chromatin regulator and the transcription factor sets respectively).

Validation of shRNAs

Lentiviral Production and Infection

Lentivirus was produced according to Open Biosystems Trans-lentiviral shRNA Packaging System (TLP4614). The shRNA constructs targeting Med1, Med12, Med15, Smc1a, Smc3, Nipb1, Oct4, Stat3 and Tcf3 are listed below. All are available, including sequences from Open Biosystems. The shRNA targeting GFP (TRCN0000072201, Hairpin Sequence: gtcgagctggacggcgacgta) was one of the negative controls for the screen.

| | |
|---|---|
| Smc1a #1 | TRCN0000109033 |
| Smc1a #2 | TRCN0000109034 |
| Smc3 #1 | TRCN0000109009 |
| Smc3 #2 | TRCN0000109007 |
| Nipbl #1 | TRCN0000124037 |
| Nipbl #2 | TRCN0000124036 |
| Med12 #1 | TRCN0000096467 |
| Med12 #2 | TRCN0000096466 |
| Med15 #1 | TRCN0000175270 |
| Med15 #2 | TRCN0000175823 |
| Med1 #1 | TRCN0000099578 |
| Oct4 | TRCN0000009613 |
| Stat3 | TRCN0000071454 |
| Tcf3 | TRCN0000095454 |

For validation of the mediator and cohesin shRNAs, mES cells were split off MEFs, placed in a tissue culture dish for 45 minutes to selectively remove the MEFs and then plated in 6-well plates (200,000 cells/well). The following day cells were infected in ESC media containing 8 μg/ml polybrene (Sigma, H926810G) and plates were spun for 30 minutes at 2150 rpm. After 24 hours the media was removed and replaced with ESC media containing 3.5 μg/mL puromycin (Sigma, P8833). ESC media with puromycin was changed daily. Five days post infection RNA or proteins were extracted or the cells were crosslinked for immunofluorescence.

Immunofluorescence

Cells were crosslinked, permeabilized and stained as described for high-throughput screening. Images were acquired on a Nikon Inverted TE300 with a Hamamatsu Orca camera. Openlab

(http://www.improvision.com/products/openlab/) was used for image acquisition. Openlab and Photoshop CS3 Extended were used for image manipulation.

RNA Extraction, cDNA, and TaqMan Expression Analysis

RNA utilized for real-time qPCR was extracted with TRIzol according to the manufacturer protocol (Invitrogen, 15596-026). Purified RNA was reverse transcribed using Superscript III (Invitrogen) with oligo dT primed first-strand synthesis following the manufacturer protocol.

Real-time qPCR were carried out on the 7000 ABI Detection System using the following TaqMan probes according to the manufacturer protocol (Applied Biosystems).

| | |
|---|---|
| Gapdh | Mm99999915_g1 |
| Med12 | Mm00804032_m1 |
| Med15 | Mm01171155_m1 |
| Smc1a | Mm01253647_m1 |
| Smc3 | Mm00484012_m1 |
| Nipbl | Mm01297461_m1 |
| Oct4 | Mm00658129_gH |

Expression levels were normalized to Gapdh levels. All knockdowns are relative to control shRNA GFP infections.

Chromatin Immunoprecipitation

Biological replicates of all ChIP-Seq datasets with the exception of mediator (Med12 and Med1) in MEFs were generated and combined for analysis. A summary of the ChIP-Seq data is contained within Supplementary Table 6.

For Med1 (CRSP1/TRAP220) occupied genomic regions, we performed ChIP-Seq experiments using Bethyl Laboratories (A300-793A) antibody. The affinity purified antibody was raised in rabbit against an epitope corresponding to amino acids 1523-1581 mapping at the C-terminus of human Med1.

For Med12 occupied genomic regions, we performed ChIP-Seq experiments using Bethyl Laboratories (A300-774A) antibody. The affinity purified antibody was raised in rabbit against an epitope corresponding to amino acids 2150-2212 mapping at the C-terminus of human Med12.

For Smc1a occupied genomic regions, we performed ChIP-Seq experiments using Bethyl Laboratories (A300-055A) affinity purified rabbit polyclonal antibody. The epitope recognized by A300-055A maps to a region between residue 1175 and the C-terminus of human Smc1a.

For Smc3 occupied genomic regions, we performed ChIP-Seq experiments using Abeam (ab9263) antibody. The affinity purified antibody was raised in rabbit against an epitope corresponding to the last 100 amino acids of the human Smc3 protein.

For TBP occupied genomic regions, we performed ChIP-Seq experiments using Abeam (ab818) antibody. The antibody was raised with a synthetic peptide, which represents amino acid residues 1-20 of human TBP.

For Pol2 occupied genomic regions, we performed ChIP-Seq experiments using Covance 8WG16 antibody. This mouse monoclonal antibody was raised against the C-terminal heptapeptide repeat region on the largest subunit of Pol2, purified from wheat germ extract.

For H3K79me2 occupied genomic regions, we performed ChIP-Seq experiments using Abeam ab3594 rabbit polyclonal antibody. The antibody was raised with a synthetic peptide that is within residues 50 to the C-terminus of Human Histone H3, dimethylated at K79.

For CTCF occupied genomic regions, we performed ChIP-Seq experiments using an Upstate 07-729 rabbit polyclonal antibody.

For Nipb1 occupied genomic regions, we performed ChIP-Seq experiments using a Bethyl A301-779A rabbit polyclonal antibody. The affinity purified antibody was raised in rabbit to a region between amino acid residues 1025 and 1075 of human Nipb1.

Protocols describing chromatin immunoprecipitation materials and methods have been previously described¹⁰. Embryonic stem cells or MEFs were grown to a final count of 5-10×10⁷ cells for each ChIP experiment. Cells were chemically crosslinked by the addition of one-tenth volume of fresh 11% formaldehyde solution for 15 minutes (ES cells) or 10 minutes (MEFs) at room temperature. Cells were rinsed twice with 1×PBS and harvested using a silicon scraper and flash frozen in liquid nitrogen. Cells were stored at −80° C. prior to use. Cells were resuspended, lysed in lysis buffers and sonicated to solubilize and shear crosslinked DNA. Sonication conditions vary depending on cells, culture conditions, crosslinking and equipment.

For Nipb1, Smc1a, Smc3, Pol2, H3K79me2 and Med1 the sonication buffer was 20 mM Tris-HCl pH8, 150 mM NaCl, 2 mM EDTA, 0.1% SDS, 1% Triton X-100. We used a Misonix Sonicator 3000 and sonicated at approximately 24 watts for 10×30 second pulses (60 second pause between pulses). Samples were kept on ice at all times. The resulting whole cell extract was incubated overnight at 4° C. with 100 μl of Dynal Protein G magnetic beads that had been pre-incubated with approximately 10 μg of the appropriate antibody. Beads were washed 1× with the sonication buffer, 1× with 20 mM Tris-HCl pH8, 500 mM NaCl, 2 mM EDTA, 0.1% SDS, 1% Triton X-100, 1× with 10 mM Tris-HCl pH8, 250 nM LiCl, 2 mM EDTA, 1% NP40 and 1× with TE containing 50 mM NaCl.

For Med12 and CTCF, the sonication buffer was 10 mM Tris-HCl pH8, 100 mM NaCl, 1 mM EDTA, 0.5 mM EGTA, 0.1% Na-Deoxycholate, 0.5% N-lauroylsarcosine. We used the same sonication and wash conditions as described above.

For TBP, the sonication buffer was 10 mM Tris-HCl pH8, 100 mM NaCl, EDTA, 0.5 mM EGTA, 0.1% Na-Deoxycholate and 0.5% N-lauroylsarcosine. We used a Misonix Sonicator 3000 and sonicated at approximately 24 watts for 10×30 second pulses (60 second pause between pulses). After Sonication, 10% Triton-X was added. After immunoprecipitation, beads were washed 4× with the RIPA buffer (50 mM Hepes-KOH pH 7.6, 500 mM LiCl, 1 mM EDTA, 1% NP40 and 0.7% Na-Deoxycholate) and 1× with TE containing 50 mM NaCl.

Bound complexes were eluted from the beads (50 mM Tris-HCl, pH 8.0, 10 mM EDTA and 1% SDS) by heating at 65° C. for 1 hour with occasional vortexing and crosslinking was reversed by overnight incubation at 65° C. Whole cell extract DNA reserved from the sonication step was also treated for crosslink reversal. Immunoprecipitated DNA and whole cell extract DNA were treated with RNaseA and Proteinase K. DNA was purified by phenol:chloroform:isoamyl alcohol extraction.

ChIP-Seq Sample Preparation and Analysis

All protocols for Illumina/Solexa sequence preparation, sequencing and quality control are provided by Illumina (http://www.illumina.com/pages.ilmn?ID=203). A brief summary of the technique and minor protocol modifications are described below.

Sample Preparation

DNA was prepared for sequencing according to a modified version of the Illumina/Solexa Genomic DNA protocol. Fragmented DNA was prepared for ligation of Solexa linkers by repairing the ends and adding a single adenine nucleotide overhang to allow for directional ligation. A 1:100 dilution of the Adaptor Oligo Mix (Illumina) was used in the ligation step. A subsequent PCR step with limited (18) amplification cycles added additional linker sequence to the fragments to prepare them for annealing to the Genome Analyzer flow-cell. After amplification, a narrow range of fragment sizes was selected by separation on a 2% agarose gel and excision of a band between 150-350 bp (representing shear fragments between 50 and 250 nt in length and ˜100 bp of primer sequence). The DNA was purified from the agarose and diluted to 10 nM for loading on the flow cell.

Polony Generation and Sequencing

The DNA library (2-4 pM) was applied to the flow-cell (8 samples per flow-cell) using the Cluster Station device from Illumina. The concentration of library applied to the flow-cell was calibrated such that polonies generated in the bridge amplification step originate from single strands of DNA. Multiple rounds of amplification reagents were flowed across the cell in the bridge amplification step to generate polonies of approximately 1,000 strands in 1 μm diameter spots. Double stranded polonies were visually checked for density and morphology by staining with a 1:5000 dilution of SYBR Green I (Invitrogen) and visualizing with a microscope under fluorescent illumination. Validated flow-cells were stored at 4° C. until sequencing.

Flow-cells were removed from storage and subjected to linearization and annealing of sequencing primer on the Cluster Station. Primed flow-cells were loaded into the Illumina Genome Analyzer 1G. After the first base was incorporated in the Sequencing-by-Synthesis reaction the process was paused for a key quality control checkpoint. A small section of each lane was imaged and the average intensity value for all four bases was compared to minimum thresholds. Flow-cells with low first base intensities were re-primed and if signal was not recovered the flow-cell was aborted. Flow-cells with signal intensities meeting the minimum thresholds were resumed and sequenced for 26 or 32 cycles.

ChIP-Seq Data Analysis

Images acquired from the Illumina/Solexa sequencer were processed through the bundled Solexa image extraction pipeline, which identified polony positions, performed base-calling and generated QC statistics. Sequences were aligned using ELAND software to NCBI Build 36 (UCSC mm8) of the mouse genome. Only sequences that mapped uniquely to the genome with zero or one mismatch were used for further analysis. When multiple reads mapped to the same genomic position, a maximum of two reads mapping to the same position were used. A summary of the total number of ChIP-Seq reads that were used in each experiment is provided (Supplementary Table 6), ChIP-Seq datasets profiling the genomic occupancy of H3K79me2¹¹, Oct4¹¹, Sox2¹¹, Nanog¹¹, RNA polymerase II¹² and CTCF¹³ in mES cells were obtained from previous publications and reanalyzed using the methods described below.

Analysis methods were derived from previously published methods^{11,14-16}. Sequence reads from multiple flow cells for each IP target and/or biological replicates were combined. For all datasets, excluding Pol2 and H3K79me2, each read was extended 200 bp, towards the interior of the sequenced fragment, based on the strand of the alignment. For Pol2 and H3K79me2 datasets, each read was extended 600 bp towards the interior and 400 bp towards the exterior of the sequenced fragment, based on the strand of the alignment. Across the genome, in 25 bp bins, the number of extended ChIP-Seq reads was tabulated. The 25 bp genomic bins that contained statistically significant ChIP-Seq enrichment were identified by comparison to a Poissonian background model. Assuming background reads are spread randomly throughout the genome, the probability of observing a given number of reads in a 1 kb window can be modeled as a Poisson process in which the expectation can be estimated as the number of mapped reads multiplied by the number of bins (40) into which each read maps, divided by the total number of bins available (we estimated 70%). Enriched bins within 200 bp of one another were combined into regions.

The Poissonian background model assumes a random distribution of background reads, however we have observed significant deviations from this expectation. Some of these non-random events can be detected as sites of apparent enrichment in negative control DNA samples and can create many false positives in ChIP-Seq experiments. To remove these regions, we compared genomic bins and regions that meet the statistical threshold for enrichment to a set of reads obtained from Solexa sequencing of DNA from whole cell extract (WCE) in matched cell samples. We required that enriched bins and enriched regions have five-fold greater ChIP-Seq density in the specific IP sample, compared with the control sample, normalized to the total number of reads in each dataset. This served to filter out genomic regions that are biased to having a greater than expected background density of ChIP-Seq reads. A summary of the enriched genomic regions (P-val<10⁻⁹) and genes (P-val<10⁻⁹) for each antibody is provided (Supplementary Table 4 and 5). Genomic coordinates for Supplementary Tables 4 and 5 are build NCBI36/mm8.

ChIP-Seq Density Map (Supplementary FIG. 4)

Genes were aligned with each other according to the position and direction of their transcription start site. For each experiment, the ChIP-Seq density profiles were normalized to the density per million total reads. Genes were sorted as by maximum level of Pol2 enrichment.

ChIP-Seq Enriched Region Maps (FIG. 2c and FIG. 5a, b)

The visualization shows the location of enriched regions (P-val<10⁻⁹, Supplementary Table 4) in a collection of datasets (query datasets, indicated on the top) in relation to the enriched regions of another dataset (base dataset, indicated on the y-axis). For each of the enriched regions in the base dataset, corresponding genomic regions were calculated as +/−5 kb from the center of that enriched region (one genomic region per enriched region, row). For each of these genomic regions, the location and length of any enriched regions in the query datasets were drawn.

Assigning ChIP-Seq Enriched Regions to Genes (Supplementary Table 5)

The complete set of RefSeq genes was downloaded from the UCSC table browser (http://genome.ucsc.edu/cgi-bin/hgTables?command=start) on Dec. 20, 2008. For all datasets, excluding Pol2 and H3K79me2, genes with enriched regions (P-val<10⁻⁹) within 10 kb of their transcription start site, or within the gene body were called bound. For Pol2 and H3K79me2 datasets, genes with enriched regions (P-val<10⁻⁹) within the gene body were called bound. See Supplementary Table 4 for the enriched genomic regions (P-val<10⁻⁹).

Note Regarding Summary of Occupied Genes Table (Supplementary Table 5)

Supplementary Table 5 provides binding information on every entry in the RefSeq table downloaded on Dec. 20, 2008 (See ChIP-Seq analysis above) and the bound gene numbers reflect counts of these entries. It should be noted however, that some of the gene names are not unique and thus the density map in Supplementary FIG. 4 may have fewer rows than there are entries in Supplementary Table 5.

Note Regarding Calculation of Co-occupied Regions (Supplementary Table 4)

Supplementary Table 4 contains the genomic coordinates of enriched regions (P-val<10⁻⁹) co-occupied by the indicated pair of factors. These coordinates are the union of all overlapping enriched regions of the two factors. It is possible for an enriched region of one factor to span, or bridge a gap between, two separate enriched regions of the other factor, in those cases, only one enriched region would be reported and it would be the union of all three enriched regions. This will cause the number of reported co-occupied regions to be less than the number of strictly overlapping sites reported in the Venn diagrams of FIG. 2b and Supplementary FIG. 5. The Venn diagrams are strictly the number of Smc1a sites that are partially overlapped by either CTCF, mediator (Med12) or Nipb1.

Gene Specific ChIPs

Gene specific ChIPs were performed in the indicated cell type following the protocol outlined in ChIP-Seq Sample Preparation. For the Gene specific ChIPs carried out in the knockdown cells, approximately 8×10⁶ ES cells (total) in 5×10 cm tissue culture plates were infected with the indicated shRNA as described (Validation of shRNAs) except that the plates were not spun post infection. Syber Green real-time qPCR was carried out on the 7000 ABI Detection System according to the manufacturer protocol (Applied Biosystems). Data was normalized to the whole cell extract and control regions. Primers to the genes tested and control regions are listed below and in Table S8.

Gnai2
5′-ACAGAGCGATACGGCTCAGCAA-3′
5′-AAGTGGTAGCCGAAGGCAAGTGAA-3′
Vps18
5′TCCTAGCGCCAACATGAGGAACT3′
5′-TTTCAGCCGCGAGTGTTAACTGGA-3′
Phc1
5′TTTGCTCTGCGTGACACTGAAGGT-3′
5′-AAATCCCAGCGCTTCTAGACGTAG-3′
BC0199443
5′TGCCCACGTCGTAACAAGGTTT-3′
5′AAGGCCGATCCTTTCTGGTTC-3′
Nanog
5′ATAGGGGGTGGGTAGGGTAG-3′
5′-CCCACAGAAAGAGCAAGACA-3′
Oct4
5′-TTGAACTGTGGTGGAGAGTGCT-3′
5′-TGCACCTTTGTTATGCATCTGCCG-3′
Ctrl
5′TGGGTGCCGTATGCCACATTAT-3′
5′-TTTCTGGCCATCCGCACCTTAT-3′

ChIP-Western and Co-Immunoprecipitation (FIG. 3a, b)

For ChIP-Western, same conditions as for ChIP-Seq were used. For co-immunoprecipitation, murine ES cells were harvested in cold PBS and extracted for 30 min at 4° C. in TNEN250 (50 mM Tris pH 7.5, 5 mM EDTA, 250 mM NaCl, 0.1% NP-40) with protease inhibitors. After centrifugation, supernatant was mixed to 2 volumes of TNENG (50 mM Tris pH 7.5, 5 mM EDTA, 100 mM NaCl, 0.1% NP-40, 10% glycerol). Protein complexes were immunoprecipitated overnight at 4° C. using 5 ug of Nipb1 (Bethyl, A301-779A or Rabbit IgG (Upstate, 12-370) bound to 50 ul of Dynabeads®. Immunoprecipitates were washed three times with TNEN125 (50 mM Tris pH 7.5, 5 mM EDTA, 125 mM NaCl, 0.1% NP40). For both ChIP-Western and co-immunoprecipitation, beads were boiled for 10 minutes in XT buffer (Bio-Rad) containing 100 mM DTT to elute proteins. After SDS-PAGE, Western blots were revealed with antibodies against Med23 (Bethyl, A300-425A), Smc1a (Bethyl, A300-055A), Smc3 (Abeam. Ab9236) and Nipb1 (Bethyl, A301-779A).

Protein Extraction and Western Blot Analysis (FIG. 5c and Supplementary FIG. 3a)

ES cells were lysed with CelLytic Reagent (Sigma, C2978-50 ml) containing protease inhibitors (Roche). After SDS-PAGE, Western blots were revealed with antibodies against Med1 (Bethyl, A300-793A), Med12 (Bethyl, A300-774A), Smc1a (Bethyl, A300-055A), Smc3 (Abcam, ab9263), Nipb1 (Bethyl, A301-779A) or Gapdh (Abeam, ab9484).

Mediator Affinity Purification

The mediator complex was purified from murine ES cell nuclear extracts using immobilized GST-SREBP-1a (residues 1-50)¹⁷. Bound material washed 4× with 20 column volumes of 0.5M KCl HEGN (20 mM Hepes, 0.1 mM EDTA, 10% Glycerol, 0.1% NP-40 & 0.5M KCl) buffer, 2× with 0.15M KCl HEGN buffer, and eluted. The eluted sample was further purified with a CDK8 antibody. After binding, this resin was washed 4× with 50 column volumes of 0.5M KCl HEGN buffer, 2× with 0.1M KCl HEGN buffer and eluted with 0.1M Glycine, pH 2.75. Western blot analysis was conducted with Smc3 (Abeam ab9263-50), Med15 (Taatjes Lab stock), Med12 (Bethyl A300-774A) or Nipb1 (Bethyl A301-779A) antibodies.

Chromosome Conformation Capture (3C)

3C analysis was performed essentially as described by Miele et al.¹⁸ with a few modifications. 10⁸ mES or MEF cells were crosslinked as described (ChIP-Seq Sample Preparation and Analysis). For 3C analysis performed in GFP control, Smc1a or Med12 shRNA knockdown cells, the cells were infected as described (Validation of shRNAs), except that the plates were not spun post infection. 10×10 cm tissue culture plates with approximately 1.5×10⁶ ES cells/plate were infected for each shRNA and five days post infection cells were crosslinked for 15 minutes (ChIP-Seq Sample Preparation and Analysis).

Crosslinked cells were lysed and chromatin was digested with 1000 units HaeIII (NEB) for the Nanog and Oct4 loci or 2000 units MspI (NEB) for the Phc1 and Lefty1 loci. Crosslinked fragments were subsequently ligated with 50 units T4 DNA ligase (Invitrogen) for 4 hours at 16° C. A control template was generated using a BAC clone (RP23-474F18) covering the Nanog locus, a BAC clone (RP24-352013) covering the Phc1 locus, a BAC clone (RP23-438H19) covering the Oct4 locus and a BAC clone (RP23-230B21) covering the Lefty1 locus. Ten μg of BAC DNA was digested with 2000 units HaeIII or 1800 units MspI. Random ligation of the fragments was done with 5 units T4 DNA ligase in a total volume of 60 μL. 3C primers were designed for fragments both upstream and downstream of the transcription start site within HaeIII or MspI fragments. Primers Nanog 20, Phc1 48, Oct4 346 and Lefty1 5 were used as the anchor points (Supplementary Table 7). 3C analysis was done, in which every PCR for a primer pair was done in triplicate and quantified. Each data point was corrected for PCR bias by dividing the average of three PCR signals by the average signal in the BAC control template.

Data from ES cells and MEFs were normalized to each other using the interaction frequencies between fragments in control regions (see below for primer pairs and Supplementary Table 7 for sequences). A normalization factor was determined by calculating the log ratio of each interaction frequency within the control region in ES over MEFs, followed by calculating the average of all log ratios. The raw interaction frequencies in ES were subsequently normalized to MEFs using this factor. The same normalization strategy was utilized for normalizing data from GFP control shRNA infected cells to Smc1a or Med12 knockdown ES cells. Genomic coordinates for Supplementary Table 7 are build NCBI36/mm8.

The following primer pairs were used for normalization between ES cells and MEFs for the Nanog locus (Biological Replicate 1 and 2); Acta2 11 and Acta2 16, Acta2 48 and Acta2 52, Gapdh 17 and Gapdh 19, Gapdh 17 and Gapdh 21, Gapdh 17 and Gapdh 32, Gapdh 21 and Gapdh 39, Gene Desert 5 and Gene Desert 6, Gene Desert 12 and Gene Desert 14, Gene Desert 25 and Gene Desert 26, Gene Desert 12 and Gene Desert 26.

The following primer pairs were used for normalization between ES cells and MEFs for the Phc1 locus (Biological Replicate 1); Gene Desert 0 and Gene Desert 1, Gene Desert 0 and Gene Desert 2, Gene Desert 27 and Gene Desert 28, Phc147 and Phc1 48, Phc1 48 and Phc1 49. The following primer pairs were used for normalization between ES cells and MEFs for the Phc1 locus (Biological Replicate 2); Gene Desert 0 and Gene Desert 1, Gene Desert 0 and Gene Desert 2, Gene Desert 27 and Gene Desert 28, Acta2 0 and Acta2 1, Acta2 2 and Acta2 7, Acta2 8 and Acta2 9, Acta2 0 and Acta2 13, Gapdh 0 and Gapdh 2, Gapdh 7 and Gapdh 8, Gapdh 9 and Gapdh 12, Gapdh 4 and Gapdh 12.

The following primer pairs were used for normalization between ES cells and MEFs for the Oct4 locus (Biological Replicate 1); Acta2 11 and Acta2 16, Gapdh 17 and Gapdh 19, Gapdh 17 and Gapdh 21, Gapdh 21 and Gapdh 39, Gene Desert 5 and Gene Desert 6, Gene Desert 12 and Gene Desert 14, Gene Desert 25 and Gene Desert 26, Oct4 346 and Oct4 344, Oct4 346 and Oct4 348. The following primer pairs were used for normalization between ES cells and MEFs for the Oct4 locus (Biological Replicate 2); Gapdh 17 and Gapdh 19, Gapdh 17 and Gapdh 21, Gapdh 21 and Gapdh 39, Gene Desert 5 and Gene Desert 6, Gene Desert 12 and Gene Desert 14, Gene Desert 25 and Gene Desert 26, Oct4 346 and Oct4 344, Oct4 346 and Oct4 348.

The following primer pairs were used for normalization between ES cells and MEFs for the Lefty1 locus (Biological Replicate 1 and 2); Gene Desert 0 and Gene Desert 1, Gene Desert 0 and Gene Desert 2, Gene Desert 27 and Gene Desert 28, Acta2 0 and Acta2 1, Acta2 8 and Acta2 9, Acta2 0 and Acta2 13, Gapdh 0 and Gapdh 2, Gapdh 7 and Gapdh 8, Gapdh 9 and Gapdh 12, Gapdh 4 and Gapdh 12.

The following primer pairs were used for normalization between GFP control shRNA knockdown cells and Smc1a #1 shRNA (See Validation of shRNAs) knockdown cells; Gene Desert 5 and Gene Desert 6, Gene Desert 12 and Gene Desert 14, Gene Desert 25 and Gene Desert 26, Acta2 11 and Acta2 16, Acta2 48 and Acta2 52, Gapdh 17 and Gapdh 19, Gapdh 17 and Gapdh 21, Gapdh 17 and Gapdh 32, Gapdh 21 and Gapdh 39.

The following primer pairs were used for normalization between GFP control shRNA knockdown cells and shRNA Med12 #1 (See Validation of shRNAs) knockdown cells; Gene Desert 5 and Gene Desert 6, Gene Desert 12 and Gene Desert 14, Gene Desert 25 and Gene Desert 26, Gene Desert 12 and Gene Desert 26, Acta2 11 and Acta2 16, Acta2 48 and Acta2 52, Gapdh 17 and Gapdh 19, Gapdh 17 and Gapdh 21, Gapdh 17 and Gapdh 32, Gapdh 21 and Gapdh 39.

Microarray Analysis

Information regarding the expression levels of mediator and cohesin subunits across a variety of cell types can be found at http://biogps.gnf.org¹⁹.

Cell Culture and RNA Isolation

For ES cell knockdown expression analysis, ES cells were split off MEFs, placed in a tissue culture dish for 45 minutes to selectively remove the MEFs and plated in 6-well plates. The following day cells were infected with lentiviral shRNAs targeting GFP, Smc1a #1, Med12 #1 or Nipb1 #1 (See Validation of shRNAs) in ESC media containing 8 μg/ml polybrene (Sigma, H9268-10G). After 24 hours the media was removed and replaced with ESC media containing 3.5 μg/mL puromycin (Sigma, P8833). Five days post infection RNA was isolated with TRIzol (Invitrogen, 15596-026), further purified with RNeasy columns (Qiagen, 74104) and DNase treated on column (Qiagen, 79254) following the manufacturer's protocols. RNA from two biological replicates was used for duplicate microarray expression analysis with the exception of the Nipb1 knockdown expression data.

Microarray Hybridization and Analysis

For microarray analysis, Cy3 and Cy5 labeled cRNA samples were prepared using Agilent's QuickAmp sample labeling kit starting with 1 μg total RNA. Briefly, double-stranded cDNA was generated using MMLV-RT enzyme and an oligo-dT based primer. In vitro transcription was performed using T7 RNA polymerase and either Cy3-CTP or Cy5-CTP, directly incorporating dye into the cRNA. Agilent mouse 4×44k expression arrays were hybridized according to our laboratory's standard method, which differs slightly from the standard protocol provided by Agilent. The hybridization cocktail consisted of 825 ng cy-dye labeled cRNA for each sample, Agilent hybridization blocking components, and fragmentation buffer. The hybridization cocktails were fragmented at 60° C. for 30 minutes, and then Agilent 2× hybridization buffer was added to the cocktail prior to application to the array. The arrays were hybridized for 16 hours at 60° C. in an Agilent rotor oven set to maximum speed. The arrays were treated with Wash Buffer #1 (6×SSPE/0.005% n-laurylsarcosine) on a shaking platform at room temperature for 2 minutes, and then Wash Buffer #2 (0.06×SSPE) for 2 minutes at room temperature. The arrays were then dipped briefly in acetonitrile before a final 30 second wash in Agilent Wash 3 Stabilization and Drying Solution, using a stir plate and stir bar at room temperature.

Arrays were scanned using an Agilent DNA microarray scanner. Array images were quantified and statistical significance of differential expression for each hybridization was calculated using Agilent's Feature Extraction Image Analysis software with the default two-color gene expression protocol. To calculate an average dataset from the biological replicates (Smc1a and Med12 knockdowns) the log 10 ratio values for each Agilent Feature were averaged and the log ratio p-values were multiplied (Supplementary Table 3). For each gene in our RefSeq set (see ChIP-Seq analysis section), we selected the Agilent Feature with the best average p-value that was annotated to that gene. Genes with no annotated features were reported as NA. Heatmaps were generated using log 2 ratio values according to the provided color scale.

Determining Genes Co-occupied by Smc1a, Med12 and Nipb1 with Expression Changes (FIG. 2d)

Smc1a, Med12 and Nipb1 co-occupied regions were initially mapped to a gene if the following criteria were met. The gene had evidence for Smc1a (P-val<10⁻⁹), Med12 (P-val<10⁻⁹) and Nipb1 (P-val<10⁻⁹) co-occupancy within the gene body or within 10 kb upstream of the transcriptional start site, evidence of Pol2 occupancy (P-val<10⁻⁹) within the gene body and significant (P-val<0.01) expression changes for a Smc1a, Med12 and Nipb1 knockdown in independent experiments. Expression data following a Smc1a, Med12 or Nipb1 knockdown are shown for these genes in FIG. 2d.

### ADDITIONAL REFERENCES

1. Cole, M. F., Johnstone, S. E., Newman, J. J., Kagey, M. H., & Young, R. A., Tcf3 is an integral component of the core regulatory circuitry of embryonic stem cells. Genes Dev 22 (6), 746-755 (2008).
2. Niwa, H., Miyazaki, J., & Smith, A. G., Quantitative expression of Oct-3/4 defines differentiation, dedifferentiation or self-renewal of ES cells. Nat Genet 24 (4), 372-376 (2000).
3. Hay, D. C., Sutherland, L., Clark, J., & Burdon, T., Oct-4 knockdown induces similar patterns of endoderm and trophoblast differentiation markers in human and mouse embryonic stem cells. Stem Cells 22 (2), 225-235 (2004).
4. Nichols, J. et al., Formation of pluripotent stem cells in the mammalian embryo depends on the POU transcription factor Oct4. Cell 95 (3), 379-391 (1998).
5. Pereira, L., Yi, F., & Merrill, B. J., Repression of Nanog gene transcription by Tcf3 limits embryonic stem cell self-renewal. Mol Cell Biol 26 (20), 7479-7491 (2006).
6. Kent, W. J. et al. The human genome browser at UCSC. Genome Res 12 (6), 996-1006 (2002).
7. Boyer, L. A. et al., Core transcriptional regulatory circuitry in human embryonic stem cells. Cell 122 (6), 947-956 (2005).
8. Moffat, J. et al. A lentiviral RNAi library for human and mouse genes applied to an arrayed viral high-content screen. Cell 124 (6), 1283-1298 (2006).
9. Bilodeau, S., Kagey, M. H., Frampton, G. M., Rahl, P. B., & Young, R. A., SetDB1 contributes to repression of genes encoding developmental regulators and maintenance of ES cell state. Genes Dev 23 (21), 2484-2489 (2009).
10. Boyer, L. A. et al., Polycomb complexes repress developmental regulators in murine embryonic stem cells. Nature 441 (7091), 349-353 (2006).
11. Marson, A. et al., Connecting microRNA genes to the core transcriptional regulatory circuitry of embryonic stem cells. Cell 134 (3), 521-533 (2008).
12. Seila, A. C. et al., Divergent transcription from active promoters. Science 322 (5909), 1849-1851 (2008).
13. Chen, X. et al., Integration of external signaling pathways with the core transcriptional network in embryonic stem cells. Cell 133 (6), 1106-1117 (2008).
14. Mikkelsen, T. S. et al., Genome-wide maps of chromatin state in pluripotent and lineage-committed cells. Nature 448 (7153), 553-560 (2007).
15. Johnson, D. S., Mortazavi, A., Myers, R. M., & Wold, B., Genome-wide mapping of in vivo protein-DNA interactions. Science 316 (5830), 1497-1502 (2007).
16. Guenther, M. G. et al., Aberrant chromatin at genes encoding stem cell regulators in human mixed-lineage leukemia. Genes Dev 22 (24), 3403-3408 (2008).
17. Ebmeier, C. C. & Taatjes, D. J., Activator-Mediator binding regulates Mediator-cofactor interactions. Proc Natl Acad Sci USA 107(25):11283-8 (2010).
18. Miele, A. & Dekker, J., Mapping cis- and trans-chromatin interaction networks using chromosome conformation capture (3C). Methods Mol Biol 464, 105-121 (2009).
19. Wu, C. et al., BioGPS: an extensible and customizable portal for querying and organizing gene annotation resources. Genome Biol 10 (11), R130 (2009),

SUPPLEMENTARY TABLE 2
Classification of Screen Hits

| Category | Gene Symbol | shRNAs | Z-score* |
|---|---|---|---|
| Pluripotency Controls | Oct4 (Pou5f1) | | −3.0 |
| | Stat3 | | −2.1 |
| Negative Controls | GFP | | −0.4 |
| | RFP | | 0.3 |
| Pluripotency Transcription | Esrrb | 1 | −2.8 |
| Factors | Oct4 (Pou5f1) | 3 | −2.3 |
| | Sall4 | 2 | −2.1 |
| | Sox2 | 2 | −1.9 |
| | Nanog | 1 | −1.8 |
| Mediator Complex Members | Med14 | 4 | −3.2 |
| | Med28 | 2 | −3.1 |
| | Med30 | 2 | −3.0 |
| | Med12 | 3 | −2.9 |
| | Med15 | 4 | −2.9 |
| | Med17 | 3 | −2.7 |
| | Med27 | 4 | −2.5 |
| | Med10 | 2 | −2.2 |
| | Med21 | 2 | −2.1 |
| | Med24 | 2 | −1.7 |
| | Med7 | 1 | −1.7 |
| | Med6 | 1 | −1.6 |
| Cohesin Complex Members | Smc1a | 5 | −2.9 |
| | Smc3 | 3 | −2.5 |
| | Nipbl | 3 | −1.9 |
| | Stag2 | 1 | −1.8 |
| Chromatin Regulators | Cbx7 | 1 | −2.5 |
| | Cbx8/Pc3 | 1 | −2.2 |
| | Ezh2 | 1 | −2.0 |
| Transcriptional Cofactors | Myst2 | 2 | −3.9 |
| | Myst3 | 1 | −2.9 |
| | Jmjd2c | 1 | −2.7 |
| | SetDB1 | 1 | −2.6 |
| | Cnot3 | 1 | −2.5 |
| | Chaf1a | 2 | −2.4 |
| | Ccnt2/cyclin T2 | 2 | −2.4 |
| | Sap18 | 2 | −2.2 |
| | Hdac3 | 1 | −2.2 |
| | Trim28 | 3 | −2.1 |
| | Chaf1b | 1 | −2.0 |
| | Mbd4 | 1 | −1.9 |
| | Ube2i/Ubc9 | 2 | −1.9 |
| | Ehmt1 | 1 | −1.9 |
| | Suv39h2 | 1 | −1.8 |
| | Mbd3 | 2 | −1.8 |
| | Mbd2 | 1 | −1.6 |
| | Mbd3l1 | 1 | −1.6 |
| | Sin3a | 2 | −1.5 |

*Z-score for best shRNA is shown for multiple hairpin hits

SUPPLEMENTARY TABLE 6
Summary of ChIP-Seq Data Used

| | | | | | | | Gene |
|---|---|---|---|---|---|---|---|
| | | | | | | | Expression |
| | | Total ChIP-Seq | p-value | Total Enriched | Total Genes | | Omnibus |
| Antibody/Source | Cell Type | reads | threshhold | Regions | Bound | Reference | Database ID |
| Oct4 | mES (V6.5) | 4,207,151 | 1E−09 | 21,895 | 7,600 | A. Marson et al | GSE11724 |
| Sox2 | mES (V6.5) | 8,459,555 | 1E−09 | 22,634 | 7,346 | A. Marson et al | GSE11724 |
| Nanog | mES (V6.5) | 7,632,057 | 1E−09 | 22,646 | 6,728 | A. Marson et al | GSE11724 |
| Med12 | mES (V6.5) | 19,497,386 | 1E−09 | 32,205 | 11,476 | This work | GSE22557 |
| Med1 | mES (V6.5) | 27,147,054 | 1E−09 | 33,916 | 11,796 | This work | GSE22557 |
| Nipbl | mES (V6.5) | 31,059,292 | 1E−09 | 18,572 | 9,384 | This work | GSE22557 |
| Smc1 | mES (V6.5) | 22,555,708 | 1E−09 | 43,687 | 12,644 | This work | GSE22557 |
| Smc3 | mES (V6.5) | 21,494,863 | 1E−09 | 33,005 | 10,986 | This work | GSE22557 |
| Pol2 | mES (V6.5) | 5,247,763 | 1E−09 | 15,759 | 9,246 | A. C. Seila et al | GSE12680 |
| H3K79me2 | mES (V6.5) | 4,290,704 | 1E−09 | 27,972 | 8,361 | A. Marson et al | GSE11724 |
| TBP | mES (V6.5) | 19,192,244 | 1E−09 | 18,280 | 11,496 | This work | GSE22557 |
| CTCF | mES (E14) | 4,402,282 | 1E−09 | 41,550 | 12,700 | X. Chen, et al | GSE11431 |
| Med1 | MEF | 8,000,406 | 1E−09 | 5,191 | 1,488 | This work | GSE22557 |
| Med12 | MEF | 7,523,631 | 1E−09 | 2,941 | 830 | This work | GSE22557 |
| Smc1 | MEF | 27,526,613 | 1E−09 | 34,045 | 10,453 | This work | GSE22557 |
| CTCF | MEF | 23,547,148 | 1E−09 | 16,962 | 7,670 | This work | GSE22557 |

| | | | | Gene Expression |
|---|---|---|---|---|
| | | Total ChIP-Seq | | Omnibus Database |
| Control Samples | Cell Type | reads | Reference | ID |
| Whole Cell Extract | MEF | 7,597,283 | This work | GSE22557 |
| Whole Cell Extract | mES (V6.5) | 7,041,824 | A. Marson et al | GSE11724 |
| GFP | mES (E14) | 5,137,594 | X. Chen, et al | GSE11431 |

References
Marson, A. et al., Connecting microRNA genes to the core transcriptional regulatory circuitry of embryonic stem cells. Cell 134 (3), 521-533 (2008).
Seila, A. C. et al., Divergent transcription from active promoters. Science 322 (5909), 1849-1851 (2008).
Chen, X. et al., Integration of external signaling pathways with the core transcriptional network in embryonic stem cells. 133 (6), 1106-1117 (2008).

SUPPLEMENTARY TABLE 7
Chromosome Conformation Capture (3C) Primers

| | Restriction | | | | |
|---|---|---|---|---|---|
| Primer Name | Enzyme | Chromosome | Start* | End* | Sequence |
| Nanog 2 | HaeIII | 6 | 122667866 | 122667896 | TAAAAACAGAGGCGTAGTCAGGTAAAGCAGC |
| Nanog 3 | HaeIII | 6 | 122668442 | 122668469 | GAGGGATCCATCGCCGTCTCCTAAGCAG |
| Nanog 4 | HaeIII | 6 | 122668713 | 122668742 | CTTACCAAAATTACGTCGCCCTTGGGACAC |
| Nanog 5 | HaeIII | 6 | 122669065 | 122669094 | ACCTTAGAATCCTCGAATGTTGGGCTTAGG |
| Nanog 6 | HaeIII | 6 | 122669755 | 122669786 | CGTTTAAGCAAACCACGTGAAAGACTTTTCAC |
| Nanog 7 | HaeIII | 6 | 122669954 | 122669984 | TGTATTAGTCCAGCGAATAAGCAGAAGGTAG |
| Nanog 10 | HaeIII | 6 | 122670466 | 122670493 | GGCTTAAGAGATGGGCTAGAGGGGCTGG |
| Nanog 11 | HaeIII | 6 | 122670968 | 122670998 | CAGAGGTCAACCAGCCACATTAGTTTATGTC |
| Nanog 12 | HaeIII | 6 | 122671212 | 122671244 | GGAAATGGCTGGTTTAATTATATCACACTGTTC |
| Nanog 14 | HaeIII | 6 | 122671491 | 122671519 | TTAGTGGCAATGGTAGTGGGGCAGCAGTG |
| Nanog 15 | HaeIII | 6 | 122671692 | 122671719 | CAGACAGTGGTGACGATGGTGGCAGTGG |
| Nanog 17 | HaeIII | 6 | 122672102 | 122672131 | CCAGGAAGAACCACTCCTACCAATACTCAC |
| Nanog 18 | HaeIII | 6 | 122672192 | 122672221 | ACACAGAAGCCGACTTAAGCTGGGTTAGAG |
| Nanog 19 | HaeIII | 6 | 122672409 | 122672437 | TCCATTGCTTAGACGGCTGAGGCACTTGG |
| Nanog 20*1 | HaeIII | 6 | 122673057 | 122673086 | CCCTGCAGGTGGGATTAACTGTGAATTCAC |
| Nanog 21 | HaeIII | 6 | 122673635 | 122673665 | ACCGTAGTAGTCATTAACATAAGCGGGTGTC |
| Nanog 22 | HaeIII | 6 | 122673800 | 122673829 | TCTTTGGAATATGTTCGGGGGCAGTGAGTG |
| Nanog 24 | HaeIII | 6 | 122674706 | 122674734 | AGCATTGCCATCAGCGTGGAGCACAGATG |
| Phc1 2 | MspI | 6 | 122286541 | 122286569 | ACACCCATCACTTACCTACAGAGGGGCTG |
| Phc1 5 | MspI | 6 | 122288561 | 122288588 | TCAGCCCTAGGCCGCTAGGATGTGGATG |
| Phc1 8 | MspI | 6 | 122291114 | 122291142 | GTCCGAGTCAGGTTCATGCCCACACTCTG |
| Phc1 12 | MspI | 6 | 122298120 | 122298148 | CAGTAAGIGGIGCCACTGACCTGATCTGC |
| Phc1 14 | MspI | 6 | 122300300 | 122300329 | AACCCCAGGATCACCTCCATTTGAACTAGC |
| Phc1 15 | MspI | 6 | 122304792 | 122304819 | TGTGCCCGAAGCGAGCGGACTTGGTAAG |
| Phc1 29 | MspI | 6 | 122308001 | 122308030 | TGCTACGTCTAGAAGCGCTGGGATTTGGAA |
| Phc1 30 | MspI | 6 | 122308860 | 122308888 | TATGTTCCCTAGGCCGAGAAAGCTCAGCC |
| Phc1 32 | MspI | 6 | 122310982 | 122311010 | CATGGTCTAATTAAGTATCCCTGGCCTAG |
| Phc1 37 | MspI | 6 | 122313877 | 122313906 | TGCTGTAGGTCATTCCTATTCCCCACAACC |
| Phc1 38 | MspI | 6 | 122315780 | 122315808 | TTGICACAAGTGTCGCTICTGGGTACATG |
| Phc1 39 | MspI | 6 | 122317203 | 122317230 | GCCTCTGGGTAACTCCCAACCCTTGTAC |
| Phcl 41 | MspI | 6 | 122320665 | 122320694 | AGTGGTTATCACTTCCACTAGGGCTCAAGG |
| Phc1 44 | MspI | 6 | 122321647 | 122321674 | TGCACCATCAGAGCGAGTGCTCCAAGAC |
| Phcl 47 | MspI | 6 | 122328073 | 122328102 | TGACTTCTAGTCTTACCCCCTTGTGATCAG |
| Phc1 48*¹ | MspI | 6 | 122333340 | 122333369 | CATCTACCTATGTAGTCGAGGCAACCAAGC |
| Phc1 49 | MspI | 6 | 122333847 | 122333874 | TCGTGAGCAGCCGAGGTTGGTGCCATGA |
| Phc1 52 | MspI | 6 | 122336767 | 122336796 | CTTGACAGTTGGCTATATAAGAGCATTCCT |
| Oct4 342 | HaeIII | 17 | 35111588 | 35111617 | GGCTATGTAGGGAACCCTTGAATCAAACCC |
| Oct4 343 | HaeIII | 17 | 35111805 | 35111834 | TATACTCTAGGCACGCTTAGGGCTAACCTG |
| Oct4 344 | HaeIII | 17 | 35111920 | 35111951 | TCCATAAGACAAGGTTGGTATTGAATACAGAC |
| Oct4 346*¹ | HaeIII | 17 | 35112208 | 35112236 | TTGTGAACTTGGCGGCTTCCAAGTCGCTG |
| Oct4 348 | HaeIII | 17 | 35112584 | 35112613 | CCTGATGAAGACTACCATCAAGAGACACCC |
| Oct4 349 | HaeIII | 17 | 35112687 | 35112714 | TGTCCTGGCTATGTACACTGTGGGGTGC |
| Oc14 350 | HaeIII | 17 | 35112754 | 35112783 | TCGTTCAGAGCATGGTGTAGGAGCAGACAG |
| Oct4 352 | HaeIII | 17 | 35113016 | 35113044 | AAGGGAAGCAGGGTATCTCCATCTGAGGC |
| Oct4 353 | HaeIII | 17 | 35113166 | 35113194 | AGTACTTGTTTAGGGTTAGAGCTGCCCCC |
| Oct4 355 | HaeIII | 17 | 35113297 | 35113324 | CCACCTCCCACCCGTTGGGTTTCTCCAC |
| Oct4 357 | HaeIII | 17 | 35113567 | 35113595 | GGGTCCCATGGTGTAGAGCCTCTAAACTC |
| Oct4 359 | HaeIII | 17 | 35113716 | 35113747 | GAAATAATTGGCACACGAACATTCAATGGATG |
| Oct4 361 | HaeIII | 17 | 35113974 | 35114001 | ACAGGCAGATAGCGCTCGCCTCAGTTTC |
| Oct4 362 | HaeIII | 17 | 35114053 | 35114080 | GTCAAGGCTAGAGGGTGGGATTGGGGAG |
| Oct4 363 | HaeIII | 17 | 35114165 | 35114192 | TGGCTTCAGACTTCGCCTTCTCACCCCC |
| Oct4 365 | HaeIII | 17 | 35114322 | 35114349 | ATGTCCGCCCGCATACGAGTTCTGCGGA |
| Oct4 367 | HaeIII | 17 | 35114512 | 35114540 | AAGGTGGAACCAACTCCCGAGGAGGTAAG |
| Oct4 373 | HaeIII | 17 | 35114785 | 35114814 | TGTACACCAGTGATGCGTGAAAATCAGCCC |
| Lefty1 0 | MspI | 1 | 182755732 | 182755759 | TGGTGGCGACGGGTGGACGGATGGCAGA |
| Lefty1 1 | MspI | 1 | 182756999 | 182757026 | CTGGCCTCGAACTACGAAATCCGCCTGC |
| Lefty1 2 | MspI | 1 | 182757793 | 182757822 | TTGTCAACTCTGCTCGACAAACCAGCACTG |
| Lefty1 3 | MspI | 1 | 182758986 | 182759015 | AGTGTTTGGAGGCGAAGGTAGATTATGGGC |
| Lefty1 4 | MspI | 1 | 182759718 | 182759743 | TTGCTTGGACACATGGCAGTCTCTCC |
| Lefty1 5*¹ | MspI | 1 | 182761838 | 182761867 | GAGTGTCAAACGACAATATGAGGTCAGGCC |
| Lefty1 6 | MspI | 1 | 182762956 | 182762983 | AAGAAGTGGCTCTCCCGTGTGGACCCAG |
| Lefty1 7 | MspI | 1 | 182763710 | 182763739 | ACACGGAGGCTCATGCTCATAATGTCAGCA |
| Lefty1 8 | MspI | 1 | 182764795 | 182764823 | AGAGCCTCTTCACGGTTGTGACTACAGAG |
| Lefty1 10 | MspI | 1 | 182765405 | 182765434 | CCTCCAACTCTAGAACGATCTGCCAAAGTG |
| Lefty1 13 | MspI | 1 | 182765885 | 182765913 | CTTGGCTGCACAGCGAGTGTGACCTGTAA |
| Lefty1 16 | MspI | 1 | 182768749 | 182768776 | CATACACACTGTAACCCATGCCTCTACC |
| Lefty1 17 | MspI | 1 | 182771347 | 182771374 | AACGTGAGACCTCCGCGTCGTCTCCAGG |
| Lefty1 18 | MspI | 1 | 182771684 | 182771713 | TAAAGCTGTTCCGTACCGTACCATTCCTCC |
| Lefty1 20 | MspI | 1 | 182771934 | 182771961 | ATGGTCATCCCCTCGCACGTGAGGACTC |
| Lefty1 21 | MspI | 1 | 182773129 | 182773158 | TTAAGGAATCTTGGCCATTGGTCTTGGGTC |
| Lefty1 27 | MspI | 1 | 182774329 | 182774356 | ACCCGATGCTGTCGCCAGGAGATGTACC |
| Lefty1 28 | MspI | 1 | 182775685 | 182775714 | GTCATGGTAGGATGCCAAGTATACAGAAGC |
| Lefty1 29 | MspI | 1 | 182777561 | 182777589 | GCTGGTTAGGCTTTCGTGGTAAGCGCCTT |
| Acta2 0 | MspI | 19 | 34306225 | 34306253 | TTTTGGGTTGCTGCGTCTCAAACGAGGCC |
| Acta2 1 | MspI | 19 | 34308126 | 34308155 | ACTGTGTGCAAAGACGATTGTTCCTGAACT |
| Acta2 2 | MspI | 19 | 34312537 | 34312566 | GTGTGCTCCAATTCACTTGTCAACCATCAC |
| Acta2 7 | MspI | 19 | 34315325 | 34315354 | GTTGTGCAACCTCTTTAACCCCTTAGTGTC |
| Acta2 8 | MspI | 19 | 34315731 | 34315760 | TCAGCAGGATAAACACCCTACTCAAGTGTC |
| Acta2 9 | MspI | 19 | 34318382 | 34318411 | GTCTTGTCCTCTCCGCGTTCAATGTGAATT |
| Acta2 11 | HaeIII | 19 | 34307607 | 34307636 | AGGCGCTGATCCACAAAACGTTCACAGTTG |
| Acta2 13 | MspI | 19 | 34321240 | 34321269 | AGCCTGGGAAAACTCGAAGTCATATCCCTG |
| Acta2 16 | HaeIII | 19 | 34308631 | 34308661 | TCTGAAGGGTAGGTATCCAGTGATGITCAAG |
| Acta2 48 | HaeIII | 19 | 34318381 | 34318410 | AGTCTTGTCCTCTCCGCGTTCAATGTGAAT |
| Acta2 52 | HaeIII | 19 | 34321624 | 34321653 | AGACGCAGGCACGGTTTGCACATTCCTC |
| Gapdh 0 | MspI | 6 | 125127776 | 125127805 | AGGGCACCAAACCCCCAGTTGCTCTTAAAA |
| Gapdh 2 | MspI | 6 | 125128698 | 125128727 | GGTTTTCAGGTTGCACCATATCAAGGGTGC |
| Gapdh 4 | MspI | 6 | 125129690 | 125129718 | CCTCCAAGTCCCTCGAACTAAGGGGAAAG |
| Gapdh 7 | MspI | 6 | 125130692 | 125130719 | CATCCCCGCAAAGGCGGAGTTACCAGAG |
| Gapdh 8 | MspI | 6 | 125130942 | 125130971 | AAAATGAGATTAGCGTGGCCCGAAGGACAC |
| Gapdh 9 | MspI | 6 | 125131062 | 125131089 | TCCGGCTTGCACACTTCGCACCAGCATC |
| Gapdh 12 | MspI | 6 | 125131947 | 125131976 | AAGGAGATTGCTACGCCATAGGTCAGGATG |
| Gapdh 17 | HaeIII | 6 | 125129300 | 125129331 | GCTTGGATGTACAACCCAAATATAGACTGTTC |
| Gapdh 19 | HaeIII | 6 | 125129881 | 125129910 | AATTTAACCTCAGATCAGGGCGGAGTGGAG |
| Gapdh 21 | HaeIII | 6 | 125130219 | 125130249 | AATACGCATTATGCCCGAGGACAATAAGGCT |
| Gapdh 32 | HaeIII | 6 | 125131236 | 125131265 | TGCAGTCCGTATTTATAGGAACCCGGATGG |
| Gapdh 39 | HaeIII | 6 | 125132422 | 125132451 | TTTTCGAGACCGGGATTCTTCACTCCGAAG |
| Gene Desert 0 | MspI | 3 | 147372833 | 147372862 | CAGGCAACAAACGAGAGTGTAAATCACCAC |
| Gene Desert 1 | MspI | 3 | 147376917 | 147376946 | GCTGTGGATGAGCAATGGTTGTGTTCTTCC |
| Gene Desert 2 | MspI | 3 | 147390029 | 147390058 | TGAAGGGGATACTTATGCCCCCTTGACATG |
| Gene Desert 5 | HaeIII | 3 | 147374283 | 147374311 | CCTCTCTCCGTCTACCCCTGATGGTTGTT |
| Gene Desert 6 | HaeIII | 3 | 147374873 | 147374902 | GTCCCTCCTACAAGATGCTTAAGGATATGG |
| Gene Desert 7 | MspI | 3 | 147407541 | 147407570 | AGTTACTAAAGGGTTCACTCCCTTCAGAAG |
| Gene Desert 8 | MspI | 3 | 147409918 | 147409947 | CTTTGCAAGTCTGATCTCTCAGTCTATGGC |
| Gene Desert 12 | HaeIII | 3 | 147378596 | 147378625 | CCTACGGAGACTTCGCTATGTGATTACACC |
| Gene Desert 14 | HaeIII | 3 | 147381478 | 147381506 | ACAAAAAACGAGCCGTTCCTCGATCCCCC |
| Gene Desert 25 | HaeIII | 3 | 147385166 | 147385195 | CATGGACCTCTGTGCTTTACGTTTCCTTCT |
| Gene Desert 26 | HaeIII | 3 | 147385511 | 147385539 | GAAAGAGGCATTGCGGCGATCCAGGAAAG |
| Gene Desert 27 | MspI | 3 | 147482556 | 147482583 | CAGGCAGATATTAACTAATGGGCCACTC |
| Gene Desert 28 | MspI | 3 | 147483140 | 147483168 | TGAGTTTGCTGGTGTGACGTCTGACTTGC |

*MM8 Coordinates
*¹Anchoring Primer

TABLE S8
Primers used for gene-specific ChIPs

| | |
|---|---|
| Gnai2 | 5′-ACAGAGCGATACGGCTCAGCAA-3′ |
| | (SEQ ID NO: 1) |
| | 5′-AAGTGGTAGCCGAAGGCAAGTGAA-3′ |
| | (SEQ ID NO: 2) |
| Vps18 | 5′-TCCTAGCGCCAACATGAGGAACT-3′ |
| | (SEQ ID NO: 3) |
| | 5′-TTTCAGCCGCGAGTGTTAACTGGA-3′ |
| | (SEQ ID NO: 4) |
| Phc1 | 5′-TTTGCTCTGCGTGACACTGAAGGT-3′ |
| | (SEQ ID NO: 5) |
| | 5′-AAATCCCAGCGCTTCTAGACGTAG-3′ |
| | (SEQ ID NO: 6) |
| BC0199443 | 5′-TGCCCACGTCGTAACAAGGTTT-3′ |
| | (SEQ ID NO: 7) |
| | 5′-AAGGCCGATCCTTTCTGGTTCA-3′ |
| | (SEQ ID NO: 8) |
| Nanog | 5′-ATAGGGGGTGGGTAGGGTAG-3′ |
| | (SEQ ID NO: 9) |
| | 5′-CCCACAGAAAGAGCAAGACA-3′ |
| | (SEQ ID NO: 10) |
| Oct4 | 5′-TTGAACTGTGGTGGAGAGTGCT-3′ |
| | (SEQ ID NO: 11) |
| | 5′-TGCACCTTTGTTATGCATCTGCCG-3′ |
| | (SEQ ID NO: 12) |
| Ctrl | 5′-TGGGTGCCGTATGCCACATTAT-3′ |
| | (SEQ ID NO: 13) |
| | 5′-TTTCTGGCCATCCGCACCTTAT-3′ |
| | (SEQ ID NO: 14) |

TABLE S9

| | | | Category | Z score |
|---|---|---|---|---|
| TRCN0000039402 | 433759 | Hdac1 | Chromatin Regulator | −0.3 |
| TRCN0000039403 | 433759 | Hdac1 | Chromatin Regulator | 0.3 |
| TRCN0000039401 | 433759 | Hdac1 | Chromatin Regulator | 0.5 |
| TRCN0000039399 | 433759 | Hdac1 | Chromatin Regulator | |
| TRCN0000039400 | 433759 | Hdac1 | Chromatin Regulator | |
| TRCN0000039395 | 15182 | Hdac2 | Chromatin Regulator | 0.9 |
| TRCN0000039398 | 15182 | Hdac2 | Chromatin Regulator | 1.2 |
| TRCN0000039396 | 15182 | Hdac2 | Chromatin Regulator | 1.4 |
| TRCN0000039397 | 15182 | Hdac2 | Chromatin Regulator | |
| TRCN0000039392 | 15183 | Hdac3 | Chromatin Regulator | |
| TRCN0000039391 | 15183 | Hdac3 | Chromatin Regulator | −0.9 |
| TRCN0000039390 | 15183 | Hdac3 | Chromatin Regulator | −0.4 |
| TRCN0000039389 | 15183 | Hdac3 | Chromatin Regulator | 0.8 |
| TRCN0000039251 | 208727 | Hdac4 | Chromatin Regulator | −0.3 |
| TRCN0000039252 | 208727 | Hdac4 | Chromatin Regulator | 0.3 |
| TRCN0000039253 | 208727 | Hdac4 | Chromatin Regulator | 0.3 |
| TRCN0000039249 | 208727 | Hdac4 | Chromatin Regulator | 0.4 |
| TRCN0000039386 | 15184 | Hdac5 | Chromatin Regulator | −1.1 |
| TRCN0000039385 | 15184 | Hdac5 | Chromatin Regulator | −0.4 |
| TRCN0000039388 | 15184 | Hdac5 | Chromatin Regulator | −0.2 |
| TRCN0000039384 | 15184 | Hdac5 | Chromatin Regulator | 0.0 |
| TRCN0000039387 | 15184 | Hdac5 | Chromatin Regulator | 0.7 |
| TRCN0000008414 | 15185 | Hdac6 | Chromatin Regulator | −0.6 |
| TRCN0000008416 | 15185 | Hdac6 | Chromatin Regulator | −0.5 |
| TRCN0000008417 | 15185 | Hdac6 | Chromatin Regulator | −0.4 |
| TRCN0000008415 | 15185 | Hdac6 | Chromatin Regulator | 0.1 |
| TRCN0000008418 | 15185 | Hdac6 | Chromatin Regulator | 0.1 |
| TRCN0000039335 | 56233 | Hdac7 | Chromatin Regulator | |
| TRCN0000039334 | 56233 | Hdac7 | Chromatin Regulator | −1.3 |
| TRCN0000039336 | 56233 | Hdac7 | Chromatin Regulator | −0.9 |
| TRCN0000039338 | 56233 | Hdac7 | Chromatin Regulator | 0.0 |
| TRCN0000039337 | 56233 | Hdac7 | Chromatin Regulator | 0.5 |
| TRCN0000088000 | 70315 | Hdac8 | Chromatin Regulator | 0.3 |
| TRCN0000088001 | 70315 | Hdac8 | Chromatin Regulator | 0.4 |
| TRCN0000087998 | 70315 | Hdac8 | Chromatin Regulator | 1.1 |
| TRCN0000087999 | 70315 | Hdac8 | Chromatin Regulator | |
| TRCN0000088002 | 70315 | Hdac8 | Chromatin Regulator | |
| TRCN0000176073 | 79221 | Hdac9 | Chromatin Regulator | −0.4 |
| TRCN0000175285 | 79221 | Hdac9 | Chromatin Regulator | 0.4 |
| TRCN0000174983 | 79221 | Hdac9 | Chromatin Regulator | 0.9 |
| TRCN0000174507 | 79221 | Hdac9 | Chromatin Regulator | 1.1 |
| TRCN0000175012 | 79221 | Hdac9 | Chromatin Regulator | |
| TRCN0000039254 | 170787 | Hdac10 | Chromatin Regulator | −0.7 |
| TRCN0000039258 | 170787 | Hdac10 | Chromatin Regulator | 0.0 |
| TRCN0000039256 | 170787 | Hdac10 | Chromatin Regulator | 0.1 |
| TRCN0000039257 | 170787 | Hdac10 | Chromatin Regulator | 0.8 |
| TRCN0000039255 | 170787 | Hdac10 | Chromatin Regulator | |
| TRCN0000039227 | 232232 | Hdac11 | Chromatin Regulator | −1.0 |
| TRCN0000039226 | 232232 | Hdac11 | Chromatin Regulator | 1.5 |
| TRCN0000039225 | 232232 | Hdac11 | Chromatin Regulator | |
| TRCN0000039224 | 232232 | Hdac11 | Chromatin Regulator | |
| TRCN0000039228 | 232232 | Hdac11 | Chromatin Regulator | |

TABLE S10

| At least a two fold | At least a two fold | At least a two fold |
|---|---|---|
| increase in expression | increase in expression | increase in expression |
| following a Smc1a | following a | in both a Smc1a and |
| Knockdown | Med12Knockdown | Med12 Knockdown |
| Fabp4 | Dkk1 | Dkk1 |
| Tbx18 | Il15 | Il15 |
| Rhoj | Ptprj | Ptprj |
| Frzb | Lgals1 | Lgals1 |
| Maf | Fhl2 | Fhl2 |
| Dlx2 | Acta1 | Acta1 |
| Ifi204 | Vnn1 | Vnn1 |
| Zic1 | Flt1 | Flt1 |
| Cav2 | Bmp8b | Bmp8b |
| Foxc1 | Huwe1 | Huwe1 |
| Chrdl2 | Wnt3 | Wnt3 |
| Msx1 | Clic5 | Cryab |
| Egfr | Cd4 | Rbp1 |
| Bmp2 | Vdr | Pmp22 |
| Krt8 | Cryab | Tmem176b |
| Lhx5 | Ntn4 | Egfr |
| Dhh | Rbp1 | Tbx18 |
| Prkg1 | Pmp22 | Bmp1 |
| Cryab | Tmem176b | Hoxa1 |
| Tnc | Egfr | Barx1 |
| Dkk1 | Taf7l | Krt8 |
| Fhl2 | Tbx18 | Rhoq |
| Vnn1 | Bmp1 | Gata3 |
| Il15 | Hoxa1 | Unc45b |
| Sox17 | Barx1 | Tnnt2 |
| Acta1 | Myocd | Cd24a |
| Cav1 | Mcoln3 | Prox1 |
| Pitx1 | Slc2a4 | Dmkn |
| Fzd1 | Krt8 | Igf2 |
| Jun | Rhoq | Chst11 |
| Irx5 | Gata3 | Cav2 |
| Ank1 | Scarf1 | Mycbpap |
| Pax3 | Unc45b | App |
| Bmp1 | Tnnt2 | Tbx1 |
| Lgals1 | Cd24a | Lyn |
| Tgfbr2 | Prox1 | Amot |
| Runx1 | Lrrc17 | Flnc |
| Il7 | Dmkn | Npnt |
| Gata6 | Igf2 | Nox4 |
| Alcam | Chst11 | Csf2 |
| Prox1 | Cav2 | Jak2 |
| Nr2f1 | Mycbpap | Cdx2 |
| Pappa | App | Efnb1 |
| Fas | Sema3b | Cdc42ep1 |
| Twist2 | Tbx1 | Pitx1 |
| Foxa2 | Lyn | Mbnl3 |
| Itgav | Amot | Fabp4 |
| Amot | Flnc | Sox17 |
| Igfbp5 | Npnt | Timp2 |
| Nox4 | Nox4 | Nfatc1 |
| Timp2 | Gcm1 | Peg10 |
| Csrp3 | Cd74 | Ulk2 |
| Axin2 | Csf2 | Cxcl12 |
| Shroom1 | Jak2 | Dlx2 |
| Vax2 | Cdon | Bin1 |
| Mybpc3 | Cdx2 | Rtn4rl1 |
| Fabp7 | Efnb1 | Cd83 |
| Tdrd7 | Cdc42ep1 | 9030409G11Rik |
| Tnnt2 | Pitx1 | Rhou |
| Rarb | Mbnl3 | Cdkn1c |
| Lgals3 | Fabp4 | Fzd1 |
| Wnt3 | Sox17 | Bmp8a |
| Isl2 | Zbtb7b | Dhh |
| Nr2f2 | Timp2 | Wnt9a |
| Col1a1 | Nfatc1 | Foxc1 |
| Hoxa11 | Peg10 | Myh9 |
| Flnc | Ulk2 | Speg |
| Fasl | Cxcl12 | Tdrd7 |
| Rgnef | Dlx2 | Tgfb1i1 |
| Rbp1 | Efna1 | Alcam |
| Tgfb1i1 | Bin1 | Axin2 |
| Capn2 | Rtn4rl1 | Sema3f |
| Unc45b | Cd83 | Ctgf |
| Bmp8a | 9030409G11Rik | Fas |
| Foxd1 | Irx4 | Kitl |
| Serpine2 | Fgfr2 | Pdlim7 |
| Arhgap24 | Rhou | Myo1e |
| Nkx2-9 | Cebpb | Dab2 |
| Adrb2 | Adamts9 | Tgfbr2 |
| Peg10 | Cdkn1c | Lama4 |
| Col11a1 | Fzd1 | Lhx5 |
| Casp8 | Cited1 | Sim2 |
| App | Bmp8a | Serpine2 |
| Ntf3 | Dhh | Cxadr |
| En1 | Wnt9a | Capn2 |
| Dmkn | Hip1 | Cav1 |
| Dock2 | Foxc1 | |
| Tmem176b | Crb3 | |
| Wnt2 | Bmp7 | |
| Adrb1 | Selenbp1 | |
| Dbx1 | Myh9 | |
| Hoxd9 | Erbb3 | |
| Lhx8 | Il11ra1 | |
| Foxg1 | Mrap | |
| Barx1 | Usp33 | |
| Sqstm1 | Gna13 | |
| Cdx2 | Speg | |
| Ptgs2 | Tdrd7 | |
| Rdh10 | Tgfb1i1 | |
| Rgs2 | Aspm | |
| Jak2 | Pard3 | |
| Ulk2 | Alcam | |
| Sox11 | Axin2 | |
| Gata2 | Sema3f | |
| Sema3a | Ctgf | |
| Ablim1 | Sox9 | |
| Gli3 | Fhl1 | |
| Evx1 | Fas | |
| Cdc42ep1 | Kitl | |
| Nrp1 | Pdlim7 | |
| Lyn | Myo1e | |
| Prdm6 | Ank3 | |
| Edn1 | Dab2 | |
| Mycbpap | Lama5 | |
| Cd28 | Txndc2 | |
| Dclk1 | Tgfbr2 | |
| Pmp22 | Lama4 | |
| Chl1 | Lamb3 | |
| Rufy3 | Lhx5 | |
| Lef1 | Sim2 | |
| Hoxd10 | Nobox | |
| Actc1 | Pigt | |
| Nr4a2 | Serpine2 | |
| Rhoq | Wwtr1 | |
| Cd24a | Lamb2 | |
| Bin1 | Mapk8ip3 | |
| Kitl | Whrn | |
| Chst11 | Cxadr | |
| Dlx1 | Capn2 | |
| Bmi1 | Figla | |
| Lgi4 | Cav1 | |
| Tbx1 | Hand2 | |
| Fst | | |
| Tshr | | |
| Onecut2 | | |
| Cd36 | | |
| Rhob | | |
| Alx1 | | |
| Lilrb3 | | |
| Myh9 | | |
| Wnt9a | | |
| Btg2 | | |
| Tirap | | |
| Nfatc1 | | |
| Foxa1 | | |
| Id1 | | |
| Cyp26b1 | | |
| Nkx2-6 | | |
| Dbn1 | | |
| Gpsm1 | | |
| Sim2 | | |
| Cxcl12 | | |
| Bmp8b | | |
| Fndc3b | | |
| Col2a1 | | |
| Kazald1 | | |
| Cd276 | | |
| Socs5 | | |
| Tnfrsf12a | | |
| Huwe1 | | |
| Stat5b | | |
| Sh2b3 | | |
| Nfkb2 | | |
| Impad1 | | |
| Hoxc10 | | |
| Pax1 | | |
| Tbx2 | | |
| Npnt | | |
| Rtn4r | | |
| Nrcam | | |
| Id3 | | |
| F11r | | |
| Timp1 | | |
| Sox6 | | |
| Rora | | |
| Hoxa2 | | |
| Cxadr | | |
| Helt | | |
| Rorc | | |
| Smad3 | | |
| Speg | | |
| Bmp4 | | |
| Zfp521 | | |
| Evl | | |
| Sprr1a | | |
| Prdm8 | | |
| Itgb1 | | |
| Sema6a | | |
| Lmna | | |
| Flt1 | | |
| Agrn | | |
| Ctgf | | |
| Ppl | | |
| Irf6 | | |
| Vax1 | | |
| Tgfb1 | | |
| Akt1 | | |
| Smurf1 | | |
| Socs1 | | |
| Efna5 | | |
| Nkx2-3 | | |
| Dzip1 | | |
| Il2rg | | |
| Sox4 | | |
| Vamp5 | | |
| Csf2 | | |
| Bmp6 | | |
| Napa | | |
| Pitx2 | | |
| Junb | | |
| Igf2 | | |
| Ilk | | |
| Frs2 | | |
| Spo11 | | |
| Lor | | |
| Twist1 | | |
| Lhx2 | | |
| 9030409G11Rik | | |
| Ednra | | |
| Nme5 | | |
| Gas1 | | |
| Nkx2-5 | | |
| Mef2d | | |
| Hps6 | | |
| Efnb1 | | |
| Abhd5 | | |
| Sema3e | | |
| Nhlh1 | | |
| Nrg1 | | |
| Bcl2l1 | | |
| Fn1 | | |
| Onecut1 | | |
| Mef2a | | |
| Mfn2 | | |
| Wnt4 | | |
| Dcx | | |
| Meis1 | | |
| Hoxa1 | | |
| Cartpt | | |
| Robo2 | | |
| Arhgap22 | | |
| Nab1 | | |
| Fgf10 | | |
| Cul7 | | |
| Dpysl2 | | |
| Eid1 | | |
| Nkd1 | | |
| Mgp | | |
| Gnas | | |
| Dyrk1b | | |
| Kdr | | |
| Sema3f | | |
| Cdh1 | | |
| Epha7 | | |
| Foxc2 | | |
| Smad1 | | |
| Ndel1 | | |
| Pdlim7 | | |
| Rtn4 | | |
| Psen1 | | |
| Sema6d | | |
| Gfi1 | | |
| Cdkn2a | | |
| Bmp5 | | |
| Tcf7l2 | | |
| Zfx | | |
| Cd83 | | |
| Angpt2 | | |
| Sort1 | | |
| Gdf11 | | |
| Gata3 | | |
| Ext2 | | |
| Ryk | | |
| Tgfb2 | | |
| Hoxb7 | | |
| Myo1e | | |
| Cdc42ep3 | | |
| P2rx7 | | |
| Ptprj | | |
| Slit3 | | |
| Irx3 | | |
| Lipa | | |
| Paqr7 | | |
| Itga7 | | |
| Emx2 | | |
| Nab2 | | |
| Bex1 | | |
| Spata6 | | |
| Etv6 | | |
| Hand1 | | |
| Wt1 | | |
| Fzd2 | | |
| Atp7a | | |
| Rhou | | |
| Nav1 | | |
| Ptk2 | | |
| Unc45a | | |
| Ptprz1 | | |
| Tacc2 | | |
| Neo1 | | |
| Elf5 | | |
| Sema3d | | |
| Rarres2 | | |
| Lhx6 | | |
| Mdk | | |
| Itga3 | | |
| Cdkn1c | | |
| Pik3r1 | | |
| Eda2r | | |
| Trp63 | | |
| Ptgs1 | | |
| Ptpn11 | | |
| Mbnl3 | | |
| Hmx2 | | |
| Ar | | |
| Yipf3 | | |
| Dock7 | | |
| Hmgb3 | | |
| Robo1 | | |
| Ripk2 | | |
| Cryaa | | |
| Gdf9 | | |
| Heph | | |
| Farp2 | | |
| Ndn | | |
| Shroom3 | | |
| Stat3 | | |
| Fgf9 | | |
| Col11a2 | | |
| Numb | | |
| Tmod1 | | |
| Runx2 | | |
| Cacna1f | | |
| Palmd | | |
| Ptprc | | |
| Lama4 | | |
| Pip5k1c | | |
| Kif5c | | |
| Egr1 | | |
| Tob1 | | |
| Trim54 | | |
| Syne2 | | |
| Rac1 | | |
| Dll4 | | |
| Agpat6 | | |
| Dab2 | | |
| Rtn4rl1 | | |
| Plxnb1 | | |
| Boc | | |
| Gnaq | | |
| Smad4 | | |
| Foxf1a | | |
| Chrna1 | | |
| Ccr4 | | |
| Top2b | | |
| Ttc8 | | |
| Pbx3 | | |

TABLE S11

| | | | | | | | |
|---|---|---|---|---|---|---|---|
| | | | | | | [−10 kb, txEnd [symbol] | [−10 kb, txEnd] |
| ID1 | ID2 | Chromosome | txStart | txEnd | strand | [symbol] d12&Smc1- [symbol] | Med1&Smc1-MEF |

ES specific genes

| | | | | | | | |
|---|---|---|---|---|---|---|---|
| Pou5f1 | NM_013633 | 17 | 35114091.00 | 35118830.00 | + | 1 | 0 |
| Nanog | NM_028016 | 6 | 122673186.00 | 122679397.00 | + | 1 | 0 |
| Sox2 | NM_011443 | 3 | 34841553.00 | 34844009.00 | + | 1 | 0 |
| Lefty2 | NM_177099 | 1 | 182729793.00 | 182735775.00 | + | 1 | 0 |
| Lefty1 | NM_010094 | 1 | 182771713.00 | 182775076.00 | + | 1 | 0 |
| Stat3 | NM_011486 | 11 | 100702899.00 | 100755601.00 | − | 1 | 0 |
| Mybl2 | NM_008652 | 2 | 162746075.00 | 162776128.00 | + | 1 | 0 |
| Sall4 | NM_175303 | 2 | 168439537.00 | 168458406.00 | − | 1 | 0 |
| Mycn | NM_008709 | 12 | 12962078.00 | 12967822.00 | − | 1 | 0 |
| Tcf3 | NM_001079822 | 6 | 72555888.00 | 72718465.00 | − | 1 | 0 |
| Esrrb | NM_011934 | 12 | 87250219.00 | 87410723.00 | + | 1 | 0 |
| Tbx3 | NM_011535 | 5 | 119931285.00 | 119945218.00 | + | 1 | 0 |
| Tcfcp2l1 | NM_023755 | 1 | 120455490.00 | 120512714.00 | + | 1 | 0 |
| Rif1 | NM_175238 | 2 | 51894845.00 | 51944390.00 | + | 1 | 0 |
| Dppa5a | NM_025274 | 9 | 78152737.00 | 78153883.00 | − | 1 | 0 |
| Fgf4 | NM_010202 | 7 | 144670775.00 | 144674633.00 | + | 1 | 0 |
| Nodal | NM_013611 | 10 | 60813656.00 | 60819992.00 | + | 1 | 0 |
| Tex19 | NM_028602 | 11 | 120962232.00 | 120964401.00 | + | 1 | 0 |

MEF specific

| | | | | | | | |
|---|---|---|---|---|---|---|---|
| Il1rl1 | NM_010743 | 1 | 40384307.00 | 40392689.00 | + | 0 | 1 |
| Il1rl1 | NM_001025602 | 1 | 40385253.00 | 40409958.00 | + | 0 | 1 |
| Tll1 | NM_009390 | 8 | 66906961.00 | 67098185.00 | − | 0 | 1 |
| Wisp1 | NM_018865 | 15 | 66721061.00 | 66752868.00 | + | 0 | 1 |
| Ptgs2 | NM_011198 | 1 | 151862341.00 | 151870228.00 | + | 0 | 1 |
| Hmga2 | NM_010441 | 10 | 119764334.00 | 119879995.00 | − | 0 | 1 |
| Pappa | NM_021362 | 4 | 64610534.00 | 64843869.00 | + | 0 | 1 |
| Serpine1 | NM_008871 | 5 | 137346134.00 | 137356886.00 | − | 0 | 1 |
| Cxcl5 | NM_009141 | 5 | 91834498.00 | 91836824.00 | + | 0 | 1 |
| Adam12 | NM_007400 | 7 | 133721544.00 | 134063440.00 | − | 0 | 1 |
| Ankrd1 | NM_013468 | 19 | 36177108.00 | 36184988.00 | − | 0 | 1 |
| Ccl7 | NM_013654 | 11 | 81861908.00 | 81863716.00 | + | 0 | 1 |
| Prrx1 | NM_011127 | 1 | 165081794.00 | 165150325.00 | − | 0 | 1 |
| Prrx1 | NM_175686 | 1 | 165081794.00 | 165150325.00 | − | 0 | 1 |
| Prrx1 | NM_001025570 | 1 | 165091951.00 | 165150325.00 | − | 0 | 1 |
| Col12a1 | NM_007730 | 9 | 79384675.00 | 79504362.00 | − | 0 | 1 |
| Ptx3 | NM_008987 | 3 | 66307815.00 | 66313734.00 | + | 0 | 1 |
| Loxl2 | NM_033325 | 14 | 68344557.00 | 68428775.00 | + | 0 | 1 |
| Cd109 | NM_153098 | 9 | 78401460.00 | 78501935.00 | + | 0 | 1 |
| Fgf7 | NM_008008 | 2 | 125726224.00 | 125781964.00 | + | 0 | 1 |
| Col8a1 | NM_007739 | 16 | 57545400.00 | 57675756.00 | − | 0 | 1 |
| Prrx2 | NM_009116 | 2 | 30667289.00 | 30703260.00 | + | 0 | 1 |
| Lox | NM_010728 | 18 | 52642606.00 | 52655077.00 | − | 0 | 1 |
| Ereg | NM_007950 | 5 | 92149816.00 | 92168849.00 | + | 0 | 1 |
| Ngfb | NM_001112698 | 3 | 102598988.00 | 102650074.00 | + | 0 | 1 |
| Ngfb | NM_013609 | 3 | 102598988.00 | 102650074.00 | + | 0 | 1 |
| Twist2 | NM_007855 | 1 | 93631882.00 | 93678433.00 | + | 0 | 1 |
| Prss23 | NM_029614 | 7 | 89382976.00 | 89392778.00 | − | 0 | 1 |
| Fbln2 | NM_001081437 | 6 | 91178267.00 | 91238044.00 | + | 0 | 1 |
| Fbln2 | NM_007992 | 6 | 91178267.00 | 91238044.00 | + | 0 | 1 |
| Cyr61 | NM_010516 | 3 | 145584362.00 | 145587367.00 | − | 0 | 1 |
| Prkg2 | NM_008926 | 5 | 99171569.00 | 99277381.00 | − | 0 | 1 |

[symbol]

indicates data missing or illegible when filed

TABLE S12

| CHROM | START | STOP | STRAND | ID1 | ID2 |
|---|---|---|---|---|---|
| 17 | 34861053 | 34814063 | −1 | NM_004774 | MED1 |
| 5 | 6431639 | 6425038 | −1 | NM_032286 | MED10 |
| 17 | 4581471 | 4583645 | 1 | NM_001001683 | MED11 |
| X | 70255130 | 70279029 | 1 | NM_005120 | MED12 |
| 3 | 152287365 | 152634500 | 1 | NM_053002 | MED12L |
| 17 | 57497425 | 57374747 | −1 | NM_005121 | MED13 |
| 12 | 115199526 | 114880763 | −1 | NM_015335 | MED13L |
| X | 40479748 | 40393738 | −1 | NM_004229 | MED14 |
| 22 | 19191885 | 19271919 | 1 | NM_001003891 | MED15 |
| 22 | 19191885 | 19271919 | 1 | NM_015889 | MED15 |
| 19 | 844218 | 818961 | −1 | NM_005481 | MED16 |
| 11 | 93157052 | 93186144 | 1 | NM_004268 | MED17 |
| 1 | 28528135 | 28535063 | 1 | NM_017638 | MED18 |
| 11 | 57236249 | 57227762 | −1 | NM_153450 | MED19 |
| 6 | 41996855 | 41981069 | −1 | NM_004275 | MED20 |
| 12 | 27066749 | 27073949 | 1 | NM_004264 | MED21 |
| 9 | 135204793 | 135197575 | −1 | NM_133640 | MED22 |
| 9 | 135204793 | 135197575 | −1 | NM_181491 | MED22 |
| 6 | 131991056 | 131936798 | −1 | NM_015979 | MED23 |
| 6 | 131991056 | 131949565 | −1 | NM_004830 | MED23 |
| 17 | 35464415 | 35428875 | −1 | NM_001079518 | MED24 |
| 17 | 35464415 | 35428875 | −1 | NM_014815 | MED24 |
| 19 | 55013357 | 55032049 | 1 | NM_030973 | MED25 |
| 19 | 16600015 | 16546717 | −1 | NM_004831 | MED26 |
| 9 | 133945074 | 133725319 | −1 | NM_004269 | MED27 |
| 4 | 17225370 | 17235258 | 1 | NM_025205 | MED28 |
| 19 | 44573802 | 44583043 | 1 | NM_017592 | MED29 |
| 8 | 118602210 | 118621682 | 1 | NM_080651 | MED30 |
| 17 | 6495678 | 6487356 | −1 | NM_016060 | MED31 |
| 13 | 47567241 | 47548092 | −1 | NM_014166 | MED4 |
| 14 | 70137137 | 70120709 | −1 | NM_005466 | MED6 |
| 5 | 156502364 | 156498028 | −1 | NM_001100816 | MED7 |
| 5 | 156502499 | 156498028 | −1 | NM_004270 | MED7 |
| 1 | 43628070 | 43622174 | −1 | NM_052877 | MED8 |
| 1 | 43628070 | 43622983 | −1 | NM_201542 | MED8 |
| 17 | 17321024 | 17337259 | 1 | NM_018019 | MED9 |
| 13 | 25726755 | 25876569 | 1 | NM_001260 | CDK8 |
| 6 | 100123411 | 100096983 | −1 | NM_001013399 | CCNC |
| X | 53466343 | 53417794 | −1 | NM_006306 | SMC1A |
| 22 | 44188164 | 44118608 | −1 | NM_148674 | SMC1B |
| 10 | 112317438 | 112354382 | 1 | NM_005445 | SMC3 |
| 3 | 137953935 | 137538688 | −1 | NM_005862 | STAG1 |
| X | 122922155 | 123064186 | 1 | NM_001042749 | STAG2 |
| X | 122923236 | 123064186 | 1 | NM_001042750 | STAG2 |
| X | 122923236 | 123064186 | 1 | NM_001042751 | STAG2 |
| X | 122923236 | 123064186 | 1 | NM_006603 | STAG2 |
| 7 | 99613473 | 99649946 | 1 | NM_012447 | STAG3 |
| 8 | 117956182 | 117927354 | −1 | NM_006265 | RAD21 |
| 18 | 17434691 | 17363259 | −1 | NM_052911 | ESCO1 |
| 8 | 27687976 | 27718343 | 1 | NM_001017420 | ESCO2 |
| 5 | 36912741 | 37100057 | 1 | NM_015384 | NIPBL |
| 5 | 36912741 | 37101678 | 1 | NM_133433 | NIPBL |

We claim:

1. A method of identifying a compound that modulates the interaction between Cohesin and Mediator comprising:
(a) contacting a composition comprising at least one Cohesin component and at least one Mediator component with a test compound;
(b) assessing the level of interaction between Cohesin and Mediator that occurs in the composition; and
(c) comparing the level of interaction measured in step (b) with a suitable reference value, wherein if the level of interaction measured in step (b) differs from the reference value, the test compound modulates the interaction between Cohesin and Mediator.

2. The method of claim 1, wherein the at least one Cohesin component comprises an Smc1 or Smc3 polypeptide.

3. The method of claim 1, wherein the at least one Cohesin component comprises an Smc1 polypeptide, an Smc3 polypeptide, and a Nibp1 polypeptide.

4. The method of claim 1, wherein the at least one Mediator component comprises a Med1 or a Med12 polypeptide.

5. The method of claim 1, wherein the at least one Mediator component comprises Med6, Med7, Med10, Med12, Med14, Med15, Med17, Med21, Med24, Med27, Med28 and Med30 polypeptides.

6. The method of claim 1, wherein the Cohesin component and the Mediator component are contacted with the test compound within a cell.

7. The method of claim 1, wherein the reference value is a value obtained in the absence of the test compound.

8. The method of claim 1, wherein the level of interaction is measured by a method comprising:
(i) isolating the Cohesin component or the Mediator component under conditions suitable for maintaining a Cohesin-Mediator interaction; and
(ii) measuring the extent to which isolating the Cohesin component results in isolating at least one Mediator component or measuring the extent to which isolating the Mediator component results in isolating at least one Cohesin component.

9. The method of claim 8, wherein isolating the Cohesin component or the Mediator component comprises contacting the composition with an agent that specifically binds to the Cohesin component or the Mediator component, respectively.

10. The method of claim 1, wherein the level of interaction is measured by assessing expression of a gene whose expression depends at least in part on a Cohesin-Mediator complex.

11. The method of claim 1, wherein the level of interaction is measured by detecting a DNA loop formed by Mediator and Cohesin.

12. The method of claim 1, wherein the level of interaction is measured by detecting co-occupancy of a promoter or enhancer by Mediator and Cohesin.

13. The method of claim 1, wherein the Cohesin component and the Mediator component are contacted with the test compound within a pluripotent cell, and the level of interaction is measured by detecting a loss of pluripotency (LOP) phenotype of the cell, wherein the LOP phenotype indicates that the compound disrupts interaction between Cohesin and Mediator.

14. The method of claim 1, wherein the Cohesin component or the Mediator component is a variant Cohesin component or a variant Mediator component.

15. The method of claim 1, wherein the Cohesin component or the Mediator component is a variant Cohesin component or a variant Mediator component and the variant Cohesin component or variant Mediator component is associated with a disorder.

16. The method of claim 1, wherein if the test compound modulates the interaction between Cohesin and Mediator, the test compound is a candidate compound for treatment of a disorder.

17. The method of claim 16, wherein the Cohesin component or the Mediator component is from a cell derived from a subject having the disorder.

18. The method of claim 16, wherein the Cohesin component or the Mediator component is a variant Cohesin component or a variant Mediator component, and the variant Cohesin component or variant Mediator component is associated with a disorder.

19. The method of claim 16, wherein the disorder is associated with mutations in a gene that encodes a Cohesin component or a Mediator component.

20. The method of claim 16, wherein the disorder is a developmental disorder.

21. The method of claim 16, wherein the disorder is a proliferative disorder.

22. A method of identifying a compound that affects cell state comprising the step of:
identifying a compound that modulates the interaction between Cohesin and Mediator.

23. The method of claim 22, wherein the cell state is characteristic of a cell type of interest, and the method comprises identifying a compound that modulates the interaction between Cohesin and Mediator in a cell of that cell type.

24. The method of claim 22, wherein the cell state is characteristic of a disorder.

25. The method of claim 22, wherein the cell state is characteristic of a disorder and the method comprises identifying a compound that modulates the interaction between Cohesin and Mediator in a cell derived from a subject having the disorder.

26. The method of claim 22, wherein the cell state is characteristic of a disorder, and wherein a compound identified as modulating the interaction between Cohesin and Mediator is a candidate compound for treating the disorder.

27. The method of claim 22, wherein the disorder is associated with mutations in a gene that encodes a Cohesin component or a Mediator component.

28. The method of claim 22, wherein the disorder is a developmental disorder.

29. The method of claim 22, wherein the disorder is a proliferative disorder.

30. The method of claim 22, wherein the cell state is characteristic of a cell type of interest, and the composition comprises a Cohesin component or a Mediator component from a cell of that type.

31. The method of claim 22, wherein the cell state is characteristic of a cell type of interest, and the composition comprises a cell-type specific transcription factor whose expression is characteristic of the cell type of interest.

32. The method of claim 22, wherein the Cohesin and Mediator components are contacted with the test compound within a cell of the cell type of interest.

33. The method of claim 22, wherein the Cohesin component or the Mediator component is from a cell derived from a subject suffering from a disorder of interest.

34. The method of claim 22, wherein the Cohesin component or the Mediator component is from a cell derived from a subject having a disorder of interest, wherein the disorder is a developmental disorder.

35. The method of claim 22, wherein the Cohesin component or the Mediator component is from a cell derived from a subject having a disorder of interest, wherein the disorder is a proliferative disorder.

36. The method of claim 22, wherein the cell state is characteristic of a disorder, and the composition comprises a Cohesin component and a Mediator component from a cell derived from a subject having the disorder.

37. The method of claim 22, wherein the cell state is characteristic of a disorder, and wherein a compound identified as modulating the interaction between Cohesin and Mediator is further identified as a candidate compound for treating the disorder.

38. A method of identifying a compound that modulates the function of a Cohesin-Mediator complex comprising steps of:
(a) contacting a composition comprising at least one Cohesin component and at least one Mediator component with a test compound;
(b) assessing at least one function of a Cohesin-Mediator complex
(c) comparing the function measured in step (b) with a suitable reference value, wherein if the function measured in step (b) differs from the reference value, the test compound modulates function of a Cohesin-Mediator complex.

39. The method of claim 38, wherein the at least one Cohesin component comprises an Smc1 or Smc3 polypeptide.

40. The method of claim 38, wherein the at least one Cohesin component comprises an Smc1 polypeptide, an Smc3 polypeptide, and a Nibp1 polypeptide.

41. The method of claim 38, wherein the at least one Cohesin component comprises an Smc1 polypeptide, an Smc3 polypeptide, a STAG polypeptide, and a Nibp1 polypeptide.

42. The method of claim 38, wherein the at least one Mediator component comprises a Med1 or a Med12 polypeptide.

43. The method of claim 38, wherein the at least one Mediator component comprises Med6, Med7, Med10, Med12, Med14, Med15, Med17, Med21, Med24, Med27, Med28 and Med30 polypeptides.

44. The method of claim 38, wherein the Cohesin component and the Mediator component are contacted with the test compound within a cell.

45. The method of claim 38, wherein the composition comprises a Cohesin complex and a Mediator complex.

46. The method of claim 38, wherein the reference value is a value obtained in the absence of the test compound.

47. The method of claim 38, wherein the function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway.

48. The method of claim 38, wherein the function is measured by assessing expression of a gene whose expression depends at least in part on a Cohesin-Mediator complex.

49. The method of claim 38, wherein the function is measured by detecting a DNA loop formed by Mediator and Cohesin.

50. The method of claim 38, wherein the function is measured by detecting co-occupancy of a promoter or enhancer by Mediator and Cohesin.

51. The method of claim 38, wherein the Cohesin component and the Mediator component are contacted with the test compound within a pluripotent cell, and the function is measured by detecting a loss of pluripotency (LOP) phenotype of the cell, wherein the LOP phenotype indicates that the compound modulates function of a Cohesin-Mediator complex.

52. The method of claim 38, wherein the Cohesin component or the Mediator component is a variant Cohesin component or a variant Mediator component.

53. The method of claim 38, wherein the Cohesin component or the Mediator component is a variant Cohesin component or a variant Mediator component and the variant Cohesin component or variant Mediator component is associated with a disorder.

54. The method of claim 38, wherein if the test compound modulates the interaction between Cohesin and Mediator, the test compound is a candidate compound for treatment of a disorder.

55. The method of claim 54, wherein the Cohesin component or the Mediator component is from a cell derived from a subject having the disorder.

56. The method of claim 54, wherein the Cohesin component or the Mediator component is a variant Cohesin component or a variant Mediator component, and the variant Cohesin component or variant Mediator component is associated with a disorder.

57. The method of claim 54, wherein the disorder is associated with mutations in a gene that encodes a Cohesin component or a Mediator component.

58. The method of claim 54, wherein the disorder is a developmental disorder.

59. The method of claim 54, wherein the disorder is a proliferative disorder.

60. A method of identifying a compound that affects cell state comprising the step of:
identifying a compound that modulates a function of a Cohesin-Mediator complex.

61. The method of claim 60, wherein the compound modulates the interaction between Cohesin and Mediator.

62. The method of claim 60, wherein the function is selected from the group consisting of (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway.

63. The method of claim 60, wherein the cell state is characteristic of a cell type of interest, and the method comprises identifying a compound that modulates function of a Cohesin-Mediator complex, wherein the compound optionally modulates the interaction between Cohesin and Mediator.

64. The method of claim 60, wherein the cell state is characteristic of a disorder.

65. The method of claim 60, wherein the cell state is characteristic of a disorder and the method comprises identifying a compound that modulates the interaction between Cohesin and Mediator in a cell derived from a subject having the disorder.

66. The method of claim 60, wherein the cell state is characteristic of a disorder, and wherein a compound identified as modulating the interaction between Cohesin and Mediator is a candidate compound for treating the disorder.

67. The method of claim 60, wherein the disorder is associated with mutations in a gene that encodes a Cohesin component or a Mediator component.

68. The method of claim 60, wherein the disorder is a developmental disorder.

69. The method of claim 60, wherein the disorder is a proliferative disorder.

70. The method of claim 60, wherein the cell state is characteristic of a cell type of interest, and the composition comprises a Cohesin component or a Mediator component from a cell of that type.

71. The method of claim 60, wherein the cell state is characteristic of a cell type of interest, and the composition comprises a cell-type specific transcription factor whose expression is characteristic of the cell type of interest.

72. The method of claim 60, wherein the Cohesin and Mediator components are contacted with the test compound within a cell of the cell type of interest.

73. The method of claim 60, wherein the Cohesin component or the Mediator component is from a cell derived from a subject suffering from a disorder of interest.

74. The method of claim 60, wherein the Cohesin component or the Mediator component is from a cell derived from a subject having a disorder of interest, wherein the disorder is a developmental disorder.

75. The method of claim 60, wherein the Cohesin component or the Mediator component is from a cell derived from a subject having a disorder of interest, wherein the disorder is a proliferative disorder.

76. The method of claim 60, wherein the cell state is characteristic of a disorder, and the composition comprises a Cohesin component and a Mediator component from a cell derived from a subject having the disorder.

77. The method of claim 60, wherein the cell state is characteristic of a disorder, and wherein a compound identified as modulating the interaction between Cohesin and Mediator is further identified as a candidate compound for treating the disorder.

78. A method of identifying a candidate compound for treatment of a disorder comprising the step of:
identifying a compound that modulates the function of a Cohesin-Mediator complex.

79. The method of claim 78, wherein the compound modulates an interaction between Cohesin and Mediator.

80. The method of claim 78, wherein the function is selected from the group consisting of (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway.

81. The method of claim 78, wherein the disorder is associated with mutations in a gene that encodes a Cohesin component or a Mediator component.

82. The method of claim 78, wherein the disorder is a developmental disorder.

83. The method of claim 78, wherein the disorder is a proliferative disorder.

84. A method of identifying a compound that modifies chromatin architecture comprising the step of:
identifying a compound that modulates the function of a Cohesin-Mediator complex.

85. The method of claim 84, wherein the compound modulates interaction between a Cohesin component and a Mediator component.

86. The method of claim 84, wherein the function comprises an interaction between Mediator and Cohesin or components thereof.

87. The method of claim 84, wherein the compound modifies chromatin architecture in a cell-type specific manner.

88. A method of identifying a compound that affects cell state comprising:
(a) providing a pluripotent cell that expresses a maintenance of pluripotency (MOP) gene, wherein the MOP gene is a gene whose inhibition results in at least one phenotype indicative of loss of pluripotency (LOP phenotype);
(b) contacting the cell with a test compound;
(c) inhibiting the MOP gene;
(d) determining whether the cell exhibits at least one LOP phenotype,
wherein if the cell fails to exhibit at least one LOP phenotype as compared to a suitable control, the compound affects cell state.

89. The method of claim 88, wherein the MOP gene is a gene listed in Table S2.

90. The method of claim 88, wherein the LOP phenotype of step (a) is selected from the group consisting of: (i) reduced levels of at least one transcription factor associated with ES cell pluripotency; (ii) a loss of pluripotent cell colony morphology; (iii) reduced levels of mRNAs specifying at least one transcription factor associated with ES cell pluripotency; (iv) increased expression of mRNAs encoding at least 3 developmentally important transcription factors.

91. The method of claim 90, wherein the LOP phenotype of step (d) is selected from the group consisting of: (i) reduced levels of at least one transcription factor associated with ES cell pluripotency; (ii) a loss of pluripotent cell colony morphology; (iii) reduced levels of mRNAs specifying at least one transcription factor associated with ES cell pluripotency; (iv) increased expression of mRNAs encoding at least 3 developmentally important transcription factors.

92. The method of claim 90, wherein the LOP phenotype of step (a) and step (d) are the same.

93. The method of claim 90, wherein the LOP phenotype of step (a), step (d), or both, is expression of Oct 4 protein.

94. The method of claim 90, wherein the at least one transcription factor associated with pluripotency is selected from the group consisting of Oct 4, Nanog, and Sox2.

95. The method of claim 88, wherein the cell is an ES cell.

96. The method of claim 88, wherein the cell comprises a nucleic acid that encodes a shRNA targeted to the MOP gene, wherein expression of the shRNA is inducible, and wherein inhibiting the MOP gene comprises inducing expression of the shRNA.

97. The method of claim 88, wherein the MOP gene encodes a Cohesin component.

98. The method of claim 88, wherein the MOP gene encodes a Mediator component.

99. The method of claim 88, wherein mutations in the MOP gene, or mutations in a gene that encodes a product which interacts with the product encoded by the MOP gene, are associated with a disorder.

100. The method of claim 99, wherein the disorder is a developmental disorder.

101. The method of claim 99, wherein the disorder is a hereditary disorder.

102. The method of claim 99, wherein the MOP gene encodes a Cohesin component.

103. The method of claim 99, wherein the MOP gene encodes a Mediator component.

104. The method of claim 99, wherein the compound is a candidate compound for treating the disorder.

105. The method of claim 104, wherein the MOP gene encodes a Cohesin component.

106. The method of claim 104, wherein the MOP gene encodes a Mediator component.

107. The method of claim 104, wherein the MOP gene encodes Nipb1.

108. The method of claim 104, wherein the disorder is Cornelia de Lange syndrome.

109. The method of claim 104, wherein the MOP gene encodes Nipb1 and the disorder is Cornelia de Lange syndrome.

110. The method of claim 104, wherein the MOP gene encodes Med12.

111. The method of claim 104, wherein the disorder is Opitz-Kaveggia (FG) syndrome, Lujan syndrome, schizophrenia or congenital heart failure.

112. The method of claim 104, wherein the MOP gene encodes Med12 and the disorder is Opitz-Kaveggia (FG) syndrome, Lujan syndrome, schizophrenia or congenital heart failure.

113. An isolated complex comprising a Cohesin component and a Mediator component.

114. The isolated complex of claim 113, wherein the complex is substantially free of CTCF.

115. The isolated complex of claim 113, wherein the Cohesin component or the Mediator component is a variant Cohesin component or a variant Mediator component, respectively.

116. The isolated complex of claim 113, wherein the complex is isolated from a cell derived from a subject who has a disorder of interest.

117. The isolated complex of claim 113, wherein the Cohesin component or the Mediator component is a recombinant protein.

118. The isolated complex of claim 113, wherein the Cohesin component or the Mediator component comprises a tag.

119. The isolated complex of claim 113, further comprising a cell-type specific transcription factor.

120. The isolated complex of claim 113, further comprising a DNA loop.

121. The isolated complex of claim 113, comprising a Nipb1 polypeptide.

122. The isolated complex of claim 113, comprising a Nipb1 polypeptide, a STAG polypeptide, and an Smc polypeptide.

123. The isolated complex of claim 113, comprising a Nipb1 polypeptide, a STAG polypeptide, an Smc1a polypeptide, and Smc3 polypeptide.

124. The isolated complex of claim 113, comprising multiple Mediator components.

125. A composition comprising the isolated complex of any of claims 113-124, wherein the composition is substantially free of Cohesin components that are not complexed with Mediator components.

126. The composition of claim 125, wherein the composition is substantially free of CTCF.

127. The composition of claim 125, wherein the composition is substantially free of Mediator components not complexed with Cohesin components.

128. A method of characterizing a cell comprising:
(a) isolating material comprising a Mediator component from a cell using an agent that binds to Mediator or that binds to a Mediator-associated protein; and
(b) detecting a Cohesin component in the isolated material.

129. The method of claim 128, further comprises analyzing a Cohesin component present in the isolated material.

130. The method of claim 128, wherein the Mediator component or the Cohesin component is a variant Mediator component or a variant Cohesin component, respectively.

131. The method of claim 128, wherein the Cohesin component or the Mediator component is a recombinant protein.

132. The method of claim 128, wherein the Cohesin component or the Mediator component comprises a tag.

133. The method of claim 128, wherein the cell is derived from a subject having or suspected of having a disorder of interest.

134. The method of claim 128, wherein the cell is derived from a subject having or suspected of having a disorder of interest and the method further comprises analyzing a Cohesin component present in the isolated material.

135. The method of claim 128, wherein the cell is derived from a subject having or suspected of having a disorder of interest and the method further comprises diagnosing the subject as having or not having the disorder based at least in part on the amount or properties of a Cohesin component present in the isolated material.

136. A method of characterizing a cell comprising:
(a) isolating a complex comprising a Cohesin component from a cell using an agent that binds to Cohesin or that binds to a Cohesin-associated protein; and
(b) detecting a Mediator component in the complex.

137. The method of claim 136, further comprising analyzing a Mediator component present in the isolated material.

138. The method of claim 136, wherein the Mediator component or the Cohesin component is a variant Mediator component or a variant Cohesin component, respectively.

139. The method of claim 136, wherein the Cohesin component or the Mediator component is a recombinant protein.

140. The method of claim 136, wherein the Cohesin component or the Mediator component comprises a tag.

141. The method of claim 136, wherein the cell is derived from a subject having or suspected of having a disorder of interest.

142. The method of claim 136, wherein the cell is derived from a subject having or suspected of having a disorder of interest and the method further comprises analyzing a Mediator component present in the isolated material.

143. The method of claim 136, wherein the cell is derived from a subject having or suspected of having a disorder of interest and the method further comprises diagnosing the subject as having or not having the disorder based at least in part on the amount or properties of the Mediator component detected.

144. A method of characterizing a cell derived from a subject having or suspected of having a Cohesin-associated disorder comprising the step of determining whether the cell has an alteration in a Mediator component as compared with a reference.

145. The method of claim 144, wherein the method comprises determining whether the cell has a mutation in a gene encoding a Mediator component.

146. The method of claim 144, wherein the method comprises determining whether the cell has increased or decreased expression or post-translational modification of a Mediator component.

147. The method of claim 144, wherein the method comprises determining whether the cell has altered binding of Mediator to at least one enhancer or promoter.

148. The method of claim 144, wherein the method comprises determining whether the cell has altered interaction between Mediator and Cohesin.

149. A method of characterizing a cell derived from a subject having or suspected of having a Mediator-associated disorder comprising the step of determining whether the cell has an alteration in a Cohesin component as compared with a reference.

150. The method of claim 149, wherein the method comprises determining whether the cell has a mutation in a gene encoding a Cohesin component.

151. The method of claim 149, wherein the method comprises determining whether the cell has increased or decreased expression or post-translational modification of a Cohesin component.

152. The method of claim 149, wherein the method comprises determining whether the cell has altered binding of Cohesin to at least one enhancer or promoter.

153. The method of claim 149, wherein the method comprises determining whether the cell has altered interaction between Mediator and Cohesin.

154. A method of characterizing a cell comprising:
analyzing a function of a Cohesin-Mediator complex of the cell.

155. The method of claim 154, wherein the cell is derived from a subject having a disorder of interest.

156. The method of claim 154, wherein the cell is derived from a subject having or suspected of having a Mediator-associated disorder.

157. The method of claim 154, wherein the cell is derived from a subject having or suspected of having a Cohesin-associated disorder.

158. The method of claim 154, wherein the method comprises determining whether the cell has altered function of a Cohesin-Mediator complex as compared with a reference.

159. The method of claim 154, wherein the function is selected from the group consisting of: (a) binding of a Cohesin complex to a Mediator complex; (b) occupancy of a cell type specific gene; (c) controlling expression or activity of a cell type specific gene; and (d) mediating response to a signal transduction pathway.

160. A method of modifying cell state comprising: modulating a Cohesin-Mediator function in the cell, thereby modifying cell state.

161. The method of claim 160, wherein the method comprises contacting a cell with a compound that modulates a Cohesin-Mediator function, thereby modifying cell state.

162. The method of claim 160, wherein the function is selected from the group consisting of: (a) binding of a Cohesin complex to a Mediator complex or binding of a Cohesin component to a Mediator component; (b) occupancy of a cell type specific gene; (c) controlling expression or activity of a cell type specific gene; and (d) mediating response to a signal transduction pathway.

163. The method of claim 160, wherein the state is a state associated with a disorder.

164. The method of claim 160, wherein the cell is in a proliferative state prior to being contacted with the compound.

165. The method of claim 160, wherein the cell is in a subject.

166. The method of claim 160, wherein the method comprises administering a compound to a subject, wherein the compound modulates a Cohesin-Mediator function.

167. The method of claim 160, wherein the method comprises administering a compound to a subject, wherein the compound modulates a Cohesin-Mediator function, and wherein the modulation treats a disorder.

168. A method of treating a subject in need of treatment for a disorder associated with decreased function of a transcription-specific Cohesin complex, the method comprising administering a compound that increases transcriptional activation activity of Mediator to the subject.

169. The method of claim 168, wherein the subject has a mutation in a gene encoding Smca1, Smc3, or Nipb1.

170. The method of claim 168, wherein the subject suffers from Cornelia deLange syndrome.


SEQUENCE LISTING (123 sequences)

SEQ ID NO: 1 - DNA, 22 bp, Artificial
  primer
acagagcgatacggctcagcaa

SEQ ID NO: 2 - DNA, 24 bp, Artificial
  primer
aagtggtagccgaaggcaagtgaa

SEQ ID NO: 3 - DNA, 23 bp, Artificial
  primer
tcctagcgccaacatgaggaact

SEQ ID NO: 4 - DNA, 24 bp, Artificial
  primer
tttcagccgcgagtgttaactgga

SEQ ID NO: 5 - DNA, 24 bp, Artificial
  primer
tttgctctgcgtgacactgaaggt

SEQ ID NO: 6 - DNA, 24 bp, Artificial
  primer
aaatcccagcgcttctagacgtag

SEQ ID NO: 7 - DNA, 22 bp, Artificial
  primer
tgcccacgtcgtaacaaggttt

SEQ ID NO: 8 - DNA, 22 bp, Artificial
  primer
aaggccgatcctttctggttca

SEQ ID NO: 9 - DNA, 20 bp, Artificial
  primer
atagggggtgggtagggtag

SEQ ID NO: 10 - DNA, 20 bp, Artificial
  primer
cccacagaaagagcaagaca

SEQ ID NO: 11 - DNA, 22 bp, Artificial
  primer
ttgaactgtggtggagagtgct

SEQ ID NO: 12 - DNA, 24 bp, Artificial
  primer
tgcacctttgttatgcatctgccg

SEQ ID NO: 13 - DNA, 22 bp, Artificial
  primer
tgggtgccgtatgccacattat

SEQ ID NO: 14 - DNA, 22 bp, Artificial
  primer
tttctggccatccgcaccttat

SEQ ID NO: 15 - DNA, 31 bp, Artificial
  Nanog primer
taaaaacagaggcgtagtcaggtaaagcagc

SEQ ID NO: 16 - DNA, 28 bp, Artificial
  Nanog primer
gagggatccatcgccgtctcctaagcag

SEQ ID NO: 17 - DNA, 30 bp, Artificial
  Nanog primer
cttaccaaaattacgtcgcccttgggacac

SEQ ID NO: 18 - DNA, 30 bp, Artificial
  Nanog primer
accttagaatcctcgaatgttgggcttagg

SEQ ID NO: 19 - DNA, 32 bp, Artificial
  Nanog primer
cgtttaagcaaaccacgtgaaagacttttcac

SEQ ID NO: 20 - DNA, 31 bp, Artificial
  Nanog primer
tgtattagtccagcgaataagcagaaggtag

SEQ ID NO: 21 - DNA, 28 bp, Artificial
  Nanog primer
ggcttaagagatgggctagaggggctgg

SEQ ID NO: 22 - DNA, 31 bp, Artificial
  Nanog primer
cagaggtcaaccagccacattagtttatgtc

SEQ ID NO: 23 - DNA, 33 bp, Artificial
  Nanog primer
ggaaatggctggtttaattatatcacactgttc

SEQ ID NO: 24 - DNA, 29 bp, Artificial
  Nanog primer
ttagtggcaatggtagtggggcagcagtg

SEQ ID NO: 25 - DNA, 28 bp, Artificial
  Nanog primer
cagacagtggtgacgatggtggcagtgg

SEQ ID NO: 26 - DNA, 30 bp, Artificial
  Nanog primer
ccaggaagaaccactcctaccaatactcac

SEQ ID NO: 27 - DNA, 30 bp, Artificial
  Nanog primer
acacagaagccgacttaagctgggttagag

SEQ ID NO: 28 - DNA, 29 bp, Artificial
  Nanog primer
tccattgcttagacggctgaggcacttgg

SEQ ID NO: 29 - DNA, 30 bp, Artificial
  Nanog primer
ccctgcaggtgggattaactgtgaattcac

SEQ ID NO: 30 - DNA, 31 bp, Artificial
  Nanog primer
accgtagtagtcattaacataagcgggtgtc

SEQ ID NO: 31 - DNA, 30 bp, Artificial
  Nanog primer
tctttggaatatgttcgggggcagtgagtg

SEQ ID NO: 32 - DNA, 29 bp, Artificial
  Nanog primer
agcattgccatcagcgtggagcacagatg

SEQ ID NO: 33 - DNA, 29 bp, Artificial
  Phc primer
acacccatcacttacctacagaggggctg

SEQ ID NO: 34 - DNA, 28 bp, Artificial
  Phc primer
tcagccctaggccgctaggatgtggatg

SEQ ID NO: 35 - DNA, 29 bp, Artificial
  Phc primer
gtccgagtcaggttcatgcccacactctg

SEQ ID NO: 36 - DNA, 29 bp, Artificial
  Phc primer
cagtaagtggtgccactgacctgatctgc

SEQ ID NO: 37 - DNA, 30 bp, Artificial
  Phc primer
aaccccaggatcacctccatttgaactagc

SEQ ID NO: 38 - DNA, 28 bp, Artificial
  Phc primer
tgtgcccgaagcgagcggacttggtaag

SEQ ID NO: 39 - DNA, 30 bp, Artificial
  Phc primer
tgctacgtctagaagcgctgggatttggaa

SEQ ID NO: 40 - DNA, 29 bp, Artificial
  Phc primer
tatgttccctaggccgagaaagctcagcc

SEQ ID NO: 41 - DNA, 29 bp, Artificial
  Phc primer
catggtctaattaagtatccctggcctag

SEQ ID NO: 42 - DNA, 30 bp, Artificial
  Phc primer
tgctgtaggtcattcctattccccacaacc

SEQ ID NO: 43 - DNA, 29 bp, Artificial
  Phc primer
ttgtcacaagtgtcgcttctgggtacatg

SEQ ID NO: 44 - DNA, 28 bp, Artificial
  Phc primer
gcctctgggtaactcccaacccttgtac

SEQ ID NO: 45 - DNA, 30 bp, Artificial
  Phc primer
agtggttatcacttccactagggctcaagg

SEQ ID NO: 46 - DNA, 28 bp, Artificial
  Phc primer
tgcaccatcagagcgagtgctccaagac

SEQ ID NO: 47 - DNA, 30 bp, Artificial
  Phc primer
tgacttctagtcttacccccttgtgatcag

SEQ ID NO: 48 - DNA, 30 bp, Artificial
  Phc primer
catctacctatgtagtcgaggcaaccaagc

SEQ ID NO: 49 - DNA, 28 bp, Artificial
  Phc primer
tcgtgagcagccgaggttggtgccatga

SEQ ID NO: 50 - DNA, 30 bp, Artificial
  Phc primer
cttgacagttggctatataagagcattcct

SEQ ID NO: 51 - DNA, 30 bp, Artificial
  Oct4 primer
ggctatgtagggaacccttgaatcaaaccc

SEQ ID NO: 52 - DNA, 30 bp, Artificial
  Oct4 primer
tatactctaggcacgcttagggctaacctg

SEQ ID NO: 53 - DNA, 32 bp, Artificial
  Oct4 primer
tccataagacaaggttggtattgaatacagac

SEQ ID NO: 54 - DNA, 29 bp, Artificial
  Oct4 primer
ttgtgaacttggcggcttccaagtcgctg

SEQ ID NO: 55 - DNA, 30 bp, Artificial
  Oct4 primer
cctgatgaagactaccatcaagagacaccc

SEQ ID NO: 56 - DNA, 28 bp, Artificial
  Oct4 primer
tgtcctggctatgtacactgtggggtgc

SEQ ID NO: 57 - DNA, 30 bp, Artificial
  Oct4 primer
tcgttcagagcatggtgtaggagcagacag

SEQ ID NO: 58 - DNA, 29 bp, Artificial
  Oct4 primer
aagggaagcagggtatctccatctgaggc

SEQ ID NO: 59 - DNA, 29 bp, Artificial
  Oct4 primer
agtacttgtttagggttagagctgccccc

SEQ ID NO: 60 - DNA, 28 bp, Artificial
  Oct4 primer
ccacctcccacccgttgggtttctccac

SEQ ID NO: 61 - DNA, 29 bp, Artificial
  Oct4 primer
gggtcccatggtgtagagcctctaaactc

SEQ ID NO: 62 - DNA, 32 bp, Artificial
  Oct4 primer
gaaataattggcacacgaacattcaatggatg

SEQ ID NO: 63 - DNA, 28 bp, Artificial
  Oct4 primer
acaggcagatagcgctcgcctcagtttc

SEQ ID NO: 64 - DNA, 28 bp, Artificial
  Oct4 primer
gtcaaggctagagggtgggattggggag

SEQ ID NO: 65 - DNA, 28 bp, Artificial
  Oct4 primer
tggcttcagacttcgccttctcaccccc

SEQ ID NO: 66 - DNA, 28 bp, Artificial
  Oct4 primer
atgtccgcccgcatacgagttctgcgga

SEQ ID NO: 67 - DNA, 29 bp, Artificial
  Oct4 primer
aaggtggaaccaactcccgaggaggtaag

SEQ ID NO: 68 - DNA, 30 bp, Artificial
  Oct4 primer
tgtacaccagtgatgcgtgaaaatcagccc

SEQ ID NO: 69 - DNA, 28 bp, Artificial
  Lefty1 primer
tggtggcgacgggtggacggatggcaga

SEQ ID NO: 70 - DNA, 28 bp, Artificial
  Lefty1 primer
ctggcctcgaactacgaaatccgcctgc

SEQ ID NO: 71 - DNA, 30 bp, Artificial
  Lefty1 primer
ttgtcaactctgctcgacaaaccagcactg

SEQ ID NO: 72 - DNA, 30 bp, Artificial
  Lefty1 primer
agtgtttggaggcgaaggtagattatgggc

SEQ ID NO: 73 - DNA, 26 bp, Artificial
  Lefty1 primer
ttgcttggacacatggcagtctctcc

SEQ ID NO: 74 - DNA, 30 bp, Artificial
  Lefty1 primer
gagtgtcaaacgacaatatgaggtcaggcc

SEQ ID NO: 75 - DNA, 28 bp, Artificial
  Lefty1 primer
aagaagtggctctcccgtgtggacccag

SEQ ID NO: 76 - DNA, 30 bp, Artificial
  Lefty1 primer
acacggaggctcatgctcataatgtcagca

SEQ ID NO: 77 - DNA, 29 bp, Artificial
  Lefty1 primer
agagcctcttcacggttgtgactacagag

SEQ ID NO: 78 - DNA, 30 bp, Artificial
  Lefty1 primer
cctccaactctagaacgatctgccaaagtg

SEQ ID NO: 79 - DNA, 29 bp, Artificial
  Lefty1 primer
cttggctgcacagcgagtgtgacctgtaa

SEQ ID NO: 80 - DNA, 28 bp, Artificial
  Lefty1 primer
catacacactgtaacccatgcctctacc

SEQ ID NO: 81 - DNA, 28 bp, Artificial
  Lefty1 primer
aacgtgagacctccgcgtcgtctccagg

SEQ ID NO: 82 - DNA, 30 bp, Artificial
  Lefty1 primer
taaagctgttccgtaccgtaccattcctcc

SEQ ID NO: 83 - DNA, 28 bp, Artificial
  Lefty1 primer
atggtcatcccctcgcacgtgaggactc

SEQ ID NO: 84 - DNA, 30 bp, Artificial
  Lefty1 primer
ttaaggaatcttggccattggtcttgggtc

SEQ ID NO: 85 - DNA, 28 bp, Artificial
  Lefty1 primer
acccgatgctgtcgccaggagatgtacc

SEQ ID NO: 86 - DNA, 30 bp, Artificial
  Lefty1 primer
gtcatggtaggatgccaagtatacagaagc

SEQ ID NO: 87 - DNA, 29 bp, Artificial
  Lefty1 primer
gctggttaggctttcgtggtaagcgcctt

SEQ ID NO: 88 - DNA, 29 bp, Artificial
  Acta2 primer
ttttgggttgctgcgtctcaaacgaggcc

SEQ ID NO: 89 - DNA, 30 bp, Artificial
  Acta2 primer
actgtgtgcaaagacgattgttcctgaact

SEQ ID NO: 90 - DNA, 30 bp, Artificial
  Acta2 primer
gtgtgctccaattcacttgtcaaccatcac

SEQ ID NO: 91 - DNA, 30 bp, Artificial
  Acta2 primer
gttgtgcaacctctttaaccccttagtgtc

SEQ ID NO: 92 - DNA, 30 bp, Artificial
  Acta2 primer
tcagcaggataaacaccctactcaagtgtc

SEQ ID NO: 93 - DNA, 30 bp, Artificial
  Acta2 primer
gtcttgtcctctccgcgttcaatgtgaatt

SEQ ID NO: 94 - DNA, 30 bp, Artificial
  Acta2 primer
aggcgctgatccacaaaacgttcacagttg

SEQ ID NO: 95 - DNA, 30 bp, Artificial
  Acta2 primer
agcctgggaaaactcgaagtcatatccctg

SEQ ID NO: 96 - DNA, 31 bp, Artificial
  Acta2 primer
tctgaagggtaggtatccagtgatgttcaag

SEQ ID NO: 97 - DNA, 30 bp, Artificial
  Acta2 primer
agtcttgtcctctccgcgttcaatgtgaat

SEQ ID NO: 98 - DNA, 28 bp, Artificial
  Acta2 primer
agacgcaggcacggtttgcacattcctc

SEQ ID NO: 99 - DNA, 30 bp, Artificial
  Gapdh primer
agggcaccaaacccccagttgctcttaaaa

SEQ ID NO: 100 - DNA, 30 bp, Artificial
  Gapdh primer
ggttttcaggttgcaccatatcaagggtgc

SEQ ID NO: 101 - DNA, 29 bp, Artificial
  Gapdh primer
cctccaagtccctcgaactaaggggaaag

SEQ ID NO: 102 - DNA, 28 bp, Artificial
  Gapdh primer
catccccgcaaaggcggagttaccagag

SEQ ID NO: 103 - DNA, 30 bp, Artificial
  Gapdh primer
aaaatgagattagcgtggcccgaaggacac

SEQ ID NO: 104 - DNA, 28 bp, Artificial
  Gapdh primer
tccggcttgcacacttcgcaccagcatc

SEQ ID NO: 105 - DNA, 30 bp, Artificial
  Gapdh primer
aaggagattgctacgccataggtcaggatg

SEQ ID NO: 106 - DNA, 32 bp, Artificial
  Gapdh primer
gcttggatgtacaacccaaatatagactgttc

SEQ ID NO: 107 - DNA, 30 bp, Artificial
  Gapdh primer
aatttaacctcagatcagggcggagtggag

SEQ ID NO: 108 - DNA, 31 bp, Artificial
  Gapdh primer
aatacgcattatgcccgaggacaataaggct

SEQ ID NO: 109 - DNA, 30 bp, Artificial
  Gapdh primer
tgcagtccgtatttataggaacccggatgg

SEQ ID NO: 110 - DNA, 30 bp, Artificial
  Gapdh primer
ttttcgagaccgggattcttcactccgaag

SEQ ID NO: 111 - DNA, 30 bp, Artificial
  Gene Desert primer
caggcaacaaacgagagtgtaaatcaccac

SEQ ID NO: 112 - DNA, 30 bp, Artificial
  Gene Desert primer
gctgtggatgagcaatggttgtgttcttcc

SEQ ID NO: 113 - DNA, 30 bp, Artificial
  Gene Desert primer
tgaaggggatacttatgcccccttgacatg

SEQ ID NO: 114 - DNA, 29 bp, Artificial
  Gene Desert primer
cctctctccgtctacccctgatggttgtt

SEQ ID NO: 115 - DNA, 30 bp, Artificial
  Gene Desert primer
gtccctcctacaagatgcttaaggatatgg

SEQ ID NO: 116 - DNA, 30 bp, Artificial
  Gene Desert primer
agttactaaagggttcactcccttcagaag

SEQ ID NO: 117 - DNA, 30 bp, Artificial
  Gene Desert primer
ctttgcaagtctgatctctcagtctatggc

SEQ ID NO: 118 - DNA, 30 bp, Artificial
  Gene Desert primer
cctacggagacttcgctatgtgattacacc

SEQ ID NO: 119 - DNA, 29 bp, Artificial
  Gene Desert primer
acaaaaaacgagccgttcctcgatccccc

SEQ ID NO: 120 - DNA, 30 bp, Artificial
  Gene Desert primer
catggacctctgtgctttacgtttccttct

SEQ ID NO: 121 - DNA, 29 bp, Artificial
  Gene Desert primer
gaaagaggcattgcggcgatccaggaaag

SEQ ID NO: 122 - DNA, 28 bp, Artificial
  Gene Desert primer
caggcagatattaactaatgggccactc

SEQ ID NO: 123 - DNA, 29 bp, Artificial
  Gene Desert primer
tgagtttgctggtgtgacgtctgacttgc